(19) United States
(12) Patent Application Publication
(10) Pub. No.: US 2024/0093236 A1
(43) Pub. Date: Mar. 21, 2024

(54) RAPID HIGH THROUGHPUT SCREENING SYSTEM TO ASSESS POTENTIAL TREATMENTS FOR SPORADIC ALZHEIMER’S DISEASE

(71) Applicant: Trustees of Tufts College, MEDFORD, MA (US)

(72) Inventors: David L. Kaplan, Concord, MA (US); Dana Cairns, Somerville, MA (US)

(21) Appl. No.: 18/360,217

(22) Filed: Jul. 27, 2023

Related U.S. Application Data
(63) Continuation of application No. PCT/US22/15280, filed on Feb. 4, 2022.
(60) Provisional application No. 63/145,981, filed on Feb. 4, 2021.

(51) Int. Cl. C12N 15/86; A61K 35/30; A61P 25/28; C12N 5/0797

(52) U.S. Cl. CPC C12N 15/86; A61K 35/30; A61P 25/28; C12N 5/0623; C12N 2513/00; C12N 2710/16643; C12N 2830/008

(57) ABSTRACT

The present invention provides an in vitro model of Alzheimer's disease (AD) comprising human induced neuronal stem cells (hiNSCs) infected with herpes simplex 1 that develop an Alzheimer's disease phenotype. The invention further provides AD model comprising genetically modified HSV infected hiNSCs. The use of these in vitro AD models for high throughput screening and phenotypic analysis are also provided.

### CROSS-REFERENCE TO RELATED PATENT APPLICATIONS

This application is a bypass continuation of International Application Serial Number PCT/US2022/015280, filed Feb. 4, 2022 (2095.0441). International Application Serial Number PCT/US2022/015280 is related to and claims priority to U.S. Provisional Application No. 63/145,981, filed Feb. 4, 2021 (2095.0440). Each of the foregoing patent applications is incorporated herein by reference in their entirety for all purposes.

### STATEMENT REGARDING FEDERALLY SPONSORED RESEARCH

This invention was made with government support under Grant Number P41EB027062 awarded by the National Institutes of Health. The government has certain rights in this invention.

### SEQUENCE LISTING

A Sequence Listing accompanies this application and is submitted as an XML file of the sequence listing named “T002459 US_2095.0558_ST26.xml” which is 44 kbytes in size and was created on Nov. 28, 2023. The sequence listing is electronically submitted via Patent Center with the application and is incorporated herein by reference in its entirety.

### BACKGROUND

Alzheimer's disease (AD) is a progressive neurodegenerative disorder that causes severe cognitive decline, short-term memory loss, impaired speech, and ultimately an inability to function in day-to-day activities[1]. AD is the sixth leading cause of death in the United States, currently afflicting nearly 5.8 million Americans with an expected doubling as the population ages in the absence of medical advances to cure or at least halt the progression of this devastating disease[1]. Early onset AD (EOAD) describes patients developing the disease at age 60 or younger. This population represents approximately 1-6% of all AD cases, the majority of which are classified as familial EOAD in which multiple relatives are also diagnosed with AD and, in certain cases, demonstrate an autosomal pattern of inheritance associated with several specific gene mutations[2]. Sporadic or late-onset AD (LOAD) accounts for approximately 95% of all AD cases. These forms of AD have a complex multifactorial etiology that is not associated with a genetic predisposition and therefore remain quite elusive despite years of research.

The hallmark features of AD are the formation of neurofibrillary tangles (NFTs) and deleterious senile plaques that develop in the brain. NFTs are aggregates of the microtubule-associated protein Tau that result from aberrant hyperphosphorylation and misfolding. Senile plaques consist of extracellular accumulation of amyloid β peptide (Aβ), which is a cleavage product of the integral transmembrane protein amyloid precursor protein (APP) found in neurons. Full-length APP has been shown to be involved in neuron growth, guidance and synapse formation[3], however, APP also undergoes sequential cleavage events to generate various isoforms with different functions. During the first cleavage step of the amyloidogenic pathway, the extracellular domain of APP is cleaved by the enzyme β-secretase 1 (BACE1), which releases the soluble sAPPβ fragment into the extracellular space. A second enzyme, γ-secretase composed of multiple catalytic subunits including presenilins 1 and 2 (PSEN1/2)[4], subsequently cleaves the remaining intramembranous fragment C99 to form APP intracellular domain (AICD) and Aβ[5]. These Aβ peptides vary in size 38-43 amino acids, however, the most common isoforms are Aβ1-40, which makes up approximately 90% of resulting Aβ fragments, and Aβ1-42, which accounts for approximately 10%. This Aβ1-42 isoform can exist as a monomer or oligomer, and has the capacity to generate fibrils that aggregate and contribute to senile plaque formation[5]. Other features of AD include cell death[6], hyperactivation of glia[7], neuroinflammation[8], synaptic dysfunction and disruption of neural networks[9]. While these diagnostic criteria of AD are well defined, the cause of this disease is not well understood.

The autosomal dominant form of EOAD has been attributed to the overproduction of Aβ as the result of mutations in one of three genes: APP, PSEN1 or PSEN2[2]. Most in vitro human models of AD rely on cells derived from patients with familial EOAD, which allow for these specific gene mutation to initiate onset of disease[10]. While these models allow for the investigation of certain downstream pathways involved in the pathogenesis of this inherited type of AD (which only accounts for approximately 5% or less of all afflicted patients) they often provide little mechanistic insight into causality and potential environmental risk factors involved in the majority of AD cases.

The clinical failure of drugs for treating AD may be due, in large part, to the lack of a physiologically relevant in vitro model of this disease. Accordingly, there remains a need in the art for in vitro human models of AD that are representative of sporadic AD and LOAD.

### SUMMARY

In one aspect, the present invention provides in vitro models of Alzheimer's disease (AD) comprising a population of human induced neural stem cells (hiNSCs) infected with a low multiplicity of infection of herpes simplex virus type 1 (HSV-1). In some embodiments, the hiNSCs are grown in a monolayer culture. In other embodiments, the hiNSCs are grown in a three-dimensional biomaterial-based scaffold. In some embodiments, the hiNSCs are genetically modified to stably express a reporter gene.

In a second aspect, the present invention provides methods of generating an in vitro model of AD. The methods comprise (a) infecting a population of hiNSCs with a low multiplicity of infection of HSV-1 to produce a population of herpes-infected hiNSCs, and (b) culturing the herpes-infected hiNSCs in culture medium for at least three days, wherein the hiNSCs develop an AD-like phenotype.

In a third aspect, the present invention provides genetically modified hiNSCs comprising a viral vector encoding at least one reporter gene under the control of a synapsin promoter, wherein the expression of the one or more reporter genes indicates that the hiNSC has differentiated into a mature, functioning neuron.

In a fourth aspect, the present invention provides methods of screening for compounds that alter an AD-associated phenotype. The methods comprise (a) contacting an in vitro model of AD of the present invention with a compound; and (b) detecting a change in one or more AD-associated phenotype in the in vitro model of AD.

In as fifth aspect, the present invention provides methods of tracking and quantifying the development of AD-associated plaques in real time. The methods comprise imaging live cells within an in vitro model of AD of the present invention over time.

In a sixth aspect, the present invention provides methods of tracking and quantifying post-stimulation firing of neurons in real time. The methods comprise (a) stimulating the cells within an in vitro model of AD of the present invention with light, and (b) measuring the firing of neurons within the in vitro model of AD over time.

In as seventh aspect, the present invention provides uses of the in vitro model of AD of the present invention in a high-throughput screen for compounds that alter an AD-associated phenotype.

### BRIEF DESCRIPTION OF THE DRAWINGS

FIG. 1A-I demonstrates that hiNSCs are highly infectable and responsive to HSV-1 exposure. hiNSCs were cultured with varying MOIs for 24 hours and assayed for virus expression by immunostaining (A and B) and quantitative polymerase chain reaction (qPCR) showing viral HSV-1-UL29 expression over time (C). Results from β-III tubulin (TUJ1) and cleaved Caspase3 (CC3) immunostaining (D) indicate that relatively high MOI (˜1 or greater) results in rapid and robust cell death in 24 hours, with corresponding quantification of cell number (E) and percentage of CC3-positive cells in response to increasing MOI in hiNSCs (F). Low-level HSV-1 infection causes morphological changes. hiNSCs were cultured 1 day (G), 4 days (H), or 7 days (I) before HSV-1 exposure (MOI of 0.0001) for 2 to 3 days. Immature hiNSCs form HSV-1-positive cell conglomerates in response to infection. Scale bars, 100 m. Asterisks indicate statistically significant differences with error bars showing means±SD (*P≤0.05, **P≤0.01, and ***P≤0.001). DAPI, 4′,6-diamidino-2-phenylindole; GAPDH, glyceraldehyde-3-phosphate dehydrogenase.

FIG. 2A-I demonstrates that HSV infection causes Aβ⁺ PLFs and specific regulation of AD mediators in hiNSCs. (A) hiNSCs were cultured for 4 days then subjected to HSV-1 infection for 3 days. HSV-1-infected hiNSCs demonstrated large areas of thioflavin T (ThT)-positive regions. (B) qPCR confirms HSV-1 infection in hiNSCs. (C) Results from an ELISA against A3 isoforms Aβ1-40 and Aβ1-42, using CM from mock- or HSV-1-infected hiNSCs, reveals a statistically significant increase in Aβ1-42, but not Aβ1-40, in HSV-1-infected CM. qPCR analysis reveals significant down-regulation of APP and BACE1 (D and E) and robust up-regulation of 7-secretase subunits PSEN1/2 (F and G). (H) Further immunostaining reveals large multicellular conglomerates of Aβ⁺ PLFs that overlaps with HSV staining. (I) Those PLFs also stain positive for hyperphosphorylated Tau. Scale bars, 100 m. Asterisks indicate statistically significant differences with error bars showing means±SD (*P≤0.05 and ***P≤0.001).

FIG. 3A-J demonstrates that HSV-1 causes reactive gliosis and inflammation reminiscent of neurodegeneration in hiNSCs. (A and B) HSV-1-infected hiNSCs highly express glia marker GFAP. Immunostaining results reveal increased number of GFAP⁺ cells and an altered morphology suggestive of gliosis. Other known markers of reactive astrocytes were also up-regulated in HSV-1-infected hiNSCs: (C) vimentin, (D) LCN2, and (E) SERP3. (F and G) Pro-inflammatory marker TNFα was also up-regulated. In HSV-1-infected hiNSCs, qPCR analysis revealed similarly high expression of other inflammatory markers known to be involved in AD: (H) IL-1, (I) IL-6, and (J) IFNγ. Scale bars, 100 m. Asterisks indicate statistically significant differences with error bars showing means±SD (**P≤0.01 and ***P≤0.001).

FIG. 4A-L demonstrates that VCV treatment results in reduced AD-like phenotype induced by HSV-1 infection in hiNSCs. (A) Treatment with VCV reduces HSV-1 infection as well as generation of Aβ⁺ PLFs. (B) Quantification of infection and (C) qPCR for HSV-UL29. VCV causes a decrease in (D) total number and (E) average area of PLFs. VCV helps to normalize expression of AD mediators back to control levels in HSV-1-infected hiNSCs: (F) APP, (G) BACE1, (H) PSEN1, and (I) PSEN2. (J) VCV treatment rescues gliosis phenotype in HSV-1-infected hiNSCs. qPCR analysis reveals restoration of (K) GFAP and (L) TNFα expression. Scale bars, 100 m. Asterisks indicate statistically significant differences with error bars showing means±SD (*P≤0.05, **P≤0.01, and ***P≤0.001).

FIG. 5A-O demonstrates that hiNSCs cultured in a 3D brain model develop an AD-like phenotype in response to low-level HSV-1 infection. (A) Model of 3D human brain-like model. hiNSCs were cultured in the donut model for 4 weeks before HSV-1 infection for 1 week. (B) Low-magnification images of brain model showing β-III tubulin (TUJI1) and beta amyloid (Aβ) immunostaining. Arrows point to regions of neuronal loss in HSV-1-infected tissues. (C) Confocal images showing HSV and Aβ immunostaining in both mock- and virus-infected constructs. (D) Scanning electron micrographs reveal morphological changes in HSV-1-infected tissue constructs. Arrowheads and arrow indicate the presence of both small and relatively larger PLFs, respectively. Scale bars, 10 m. (E) APP and (F) BACE1 were down-regulated, and (G and H) PSEN1/2 were up-regulated, similar to results in monolayer cultures. HSV-1 induces gliosis and neuroinflammation in 3D cultures. Glial marker GFAP is up-regulated as evidenced by (I) immunostaining and (J) qPCR, as well as (K) pro-inflammatory marker TNFα. LFP recordings were performed to assess functionality in 3D cultures. Schematic representation of microelectrode placement for LFP assessments (L) and an image of constructs placed on electrophysiology rigs for recordings (M) (photo credit: Dana M Cairns, Tufts University). (N and O) Representative traces of mock versus infected brain models shows that HSV-1-treated donuts show significantly less activity reminiscent of impaired functionality in patients with AD. Asterisks indicate statistically significant differences with error bars showing means±SD (**P≤0.01 and ***P≤0.001).

FIG. 6 demonstrates that HSV-1-infected hiNSCs secrete HSV-1 that can infect new cells. Conditioned media (CM) was harvested and filtered from mock or HSV-1 infected hiNSCs that had been cultured for 2 days. Mock or fresh HSV-1 virus was added to CM and new hiNSCs were cultured for 4 days. Antibodies against HSV or beta amyloid fibrils (Aβ) were used to immunostain cells. Virus CM can infect new cells suggesting that infected hiNSCs secrete HSV-1. Scale bar=100 m.

FIG. 7 demonstrates that HSV-1-secreted factors cause gliosis even in the absence of infection. Conditioned media (CM) was harvested and filtered from mock or HSV-1 infected hiNSCs that had been cultured for 2 days. Fresh HSV-1 virus was added to CM in the absence of presence of antiviral treatment Valacyclovir HCl (VCV) and new hiNSCs were cultured for 4 days. Beta III tubulin (TUJ1) and glial fibrillary acidic protein (GFAP) immunostaining was performed to visualize neurons and glia, respectively. Preventing HSV-1 infection using VCV in virus CM results in increased reactive glia formation suggesting that HSV-1 induced factors play a role in astrogliosis. Arrowheads=swollen activated reactive glia; arrows=intermediate reactive glia with more fibrous extensions. Scale bar=100 m.

FIG. 8A-B demonstrates that hiNSCs cultured in 3D human brain model then subjected to low level HSV-1 infection develop AD-like plaque formations. High magnification confocal images reveal positive Thioflavin T (ThT) (A) and TAU1 (B) expression in plaque-like formations (PLFs) in HSV-1-infected 3D brain cultures. Scale bar=100 m.

FIG. 9 demonstrates that HSV-1 infected 3D human brain-like tissue constructs overexpress multiple AD-related factors. Heat map to visually portray relative expression levels of different genes in mock and virus-infected 3D human brain-like tissue cultures using the Qiagen RT Profiler Array for Alzheimer's disease. Red-green color spectrum represents relative fold change of virus-infected samples as compared to mock-controls.

FIG. 10 demonstrates that Thioflavin T dye can used to quantify plaque size over time. HSV-1-infected hiNSCs display large plaque like formations that stain positive with Thioflavin T dye. This dye can be modified to use in real time assays such as an Incucyte Live-Cell Analysis System (Sartorius).

FIG. 11 provides exemplary data tracking plaque size over time. Fluorescence emitted by bound Thioflavin T (ThT) dye (excitation emission 350 nm/440 nm) was monitored in hiNSCs treated with mock or virus (HSV-1) once per hour over the course of 60 hours. This produced a quantifiable sustained increase in percentage confluence of ThT fluorescence in AD hiNSCs only (HSV-1-infected), suggesting that this is a suitable method of quantifying amyloid plaque formation in real time.

FIG. 12 is a schematic depicting an exemplary lentiviral reporter construct used to generate optogenetically-responsive hiNSC lines for us in tissue-engineered models. This polycistronic construct contains multiple inserts that are driven by different promoters. By selecting for this construct using puromycin selection, one can generate hiNSCs that, upon differentiation, express a cherry red signal only in mature, healthy neurons.

FIG. 13 demonstrates that hiNSCs stably transduced with an exemplary lentiviral reporter construct express cherry red signal in mature, differentiating neurons.

FIG. 14 demonstrates that hiNSC lines express a puromycin resistance cassette that is driven by a constitutively active CMV promoter regardless of differentiation status. This data was generated using a qPCR analysis.

FIG. 15 shows that the expression of channelrhodopsin is only observable in hiNSC lines that were induced to differentiate, demonstrating the specificity of the synapsin promoter in channelrhodopsin and calcium sensor expression. This data was generated using a qPCR analysis.

FIG. 16 demonstrates that the increase in mature neuron-specific markers (i.e., potassium-chloride cotransporter 2 (KCC2) and synaptophysin) is correlated with synapsin promoter induced expression of channelrhodopsin. This data was generated using a qPCR analysis.

FIG. 17 shows exemplary data tracking the differentiation of neurons from HSV-1-infected hiNSCs. hiNSCs stably expressing the optogenetic reporter construct were analyzed using the Incucyte real-time imaging and analysis system. These genetically modified hiNSCs were subject to mock or virus (HSV-1) treatment, and were then imaged once per hour over the course of 60 hours. Mock-infected hiNSCs showed steady increase in total red fluorescence, while virus-infected hiNSCs did not, reflecting a lack of healthy, functioning neurons in HSV-1-infected AD-like cultures.

FIG. 18A-C demonstrates Fluo4-AM calcium imaging that reveals a decrease in functionality of HSV-1-infected 3D brain-like tissues. A) Schematic representation of the generation of silk scaffold-based 3D brain-like tissues and subsequent HSV-1 infection. B) Image of 3D brain-like tissues stained with Fluo4-AM calcium dye showing firing neurons. C) 3D tissues were imaged and processed using ImageJ. Neurons were traced and fluorescent intensity was measured over 60 sec. of imaging (N=5 neurons per treatment). Representative tracings for MOCK and VIRUS are shown.

FIG. 19A-B demonstrates an Example positive result from drug discovery assay of the instant disclosure. Compound X prevents herpes-induced AD in high throughput drug screens. Compound X prevents formation of beta amyloid-positive PLFs as evidenced by immunostaining (A) as well as reduction of presenilin-2 (PSEN2) expression using qPCR (B).

FIG. 20A-C demonstrates stable lentiviral expression of Human PSEN2-promoter driven mCherry reporter for high throughput analysis of AD-like phenotypes. A) AD-specific mediator, PSEN2, is highly upregulated in response to HSV-1 infection. B) A stable hiNSC line was generated expressing the PSEN2-promoter driven mCherry reporter. PSEN2-mCherry hiNSCs were cultured then subjected to either MOCK or HSV-1 treatment, and monitored using Incucyte real-time imaging and analysis equipment and software. HSV-1 treated hiNSCs demonstrate a quantifiable increase in PSEN2-driven mCherry reporter expression. C) Validation of PSEN2-mCherry hiNSC line using immunostaining against beta amyloid. Beta-amyloid-positive PLFs co-localize with reporter mCherry expression in HSV-1 treated hiNSCs.

FIG. 21A-E demonstrates the generation of a latency model of HSV-1 infection. A) Schematic of timeline of HSV-1 and antiviral treatments to induce latency. Acute infection of hiNSCs with HSV-1 for 24 hrs. followed by antiviral treatment results in similar expression patterns of typical AD markers as described previously (B-D). E) This treatment also results in quantifiable expression of HSV-1 latency associated transcript (LAT).

### DETAILED DESCRIPTION

The generation of in vitro models of sporadic Alzheimer's disease (AD) poses a significant challenge due to the complex, undefined etiology and slow progression of this disease. The present disclosure provides a new in vitro model of sporadic AD as described more here, and methods of use, including for screening drugs and studying the mechanism of AD disease progression.

Growing evidence implicates pathogen involvement in the development of sporadic AD[11-15]. Interestingly, the formation of Aβ1-42 monomers has actually been shown to be neuroprotective, supporting the survival of neurons against trophic deprivation and excitotoxic death[16]. It is the aggregation of these monomers into oligomers, fibrils and ultimately senile plaques, which ultimately correlates with to the neurotoxicity seen in AD. The AD pathogen hypothesis suggests that various pathogens can act as triggers to induce and/or lead to the accumulation of these Aβ1-42 monomers[14]. Many of these pathogens, such as Chlamydophila pneumoniae[11], Borrelia burgdorferi[17], and Candida glabrata[18], are able to evade the host immune response, particularly in the immunocompromised and/or the elderly, and to infiltrate the brain, forming latent and potentially chronic infections. In turn, these pathogens also go on to induce reactive gliosis[19] as well as a pro-inflammatory response[8], ultimately leading to progressive neurodegeneration and dementia.

Herpes simplex virus type I (HSV-1) is a pathogen that is gaining increasing attention as a potential causative agent in sporadic AD pathogenesis[15, 20, 21]. HSV-1 is a neurotropic double-stranded DNA virus that typically resides throughout the peripheral nervous system in a latent state. HSV-1 reactivation can be asymptomatic, manifest as cold sores, or, in rare cases, result in herpes simplex encephalitis (HSE), demonstrating the ability of the virus to penetrate the blood brain barrier[22]. Some of the earliest indications that HSV-1 might be linked to AD were the observations that acute HSV-1 infection resulting in HSE is often localized to regions of the brain most often affected by AD, with patients demonstrating cognitive and behavioral symptoms typical of AD patients[23]. Furthermore, pioneering studies from the Izthaki laboratory reported detection of high levels of herpesviridae DNA from amyloid plaques harvested from postmortem brain tissue of sporadic AD patients using PCR[24-26]. Even more recently, an epidemiological study in Taiwan reported that HSV-1 infection and seropositivity led to a significant risk of later development of AD, and that antiherpetic treatment at the time of infection dramatically reduced that risk by a factor of ten[27].

The present inventors have generated human models of sporadic AD, including both 2D and 3D human tissue models using human induced neural stem cells (hiNSCs) infected with herpes simplex virus and derived from somatic cells. The models may be used in of high throughput analyses of quantifiable AD phenotypes, such as the generation of amyloid fibril-expressing plaque-like formations (PLFs), reactive gliosis, apoptosis of neuronal cells, neuroinflammation and other AD-phenotypic characteristics.

While previous reports have suggested a potential link between HSV-1 and upregulation of certain AD mediators [31], to date, no direct causality of AD by herpes virus in a 3D human brain model has been demonstrated.

The inventors designed and have produced HSV-1 induced 2D and 3D human brain-like tissue models of AD using their hiNSCs infected with low multiplicity of infection of HSV-1. In the Examples, the inventors demonstrate that while relatively high levels of HSV-1 infection resulted in cell death in their hiNSC model (similar to what has been previously reported using other cell types [32]), low levels of the virus induced a surprising and remarkable AD-like phenotype not previously reported in vitro. Importantly, the inventors demonstrated that after as few as 3 days of HSV-1 infection (MOI 0.0001), the hiNSCs develop large, multicellular, dense Aβ+ fibrillar plaque-like formations (PLFs), reminiscent of amyloid plaques found in sporadic AD patients. HSV-1 infection also led to endogenous upregulation of the known AD mediators PSEN1 and PSEN2. Additionally, the inventors demonstrated that HSV-1 infection induced reactive gliosis and high levels of neuroinflammation, which are frequently observed in neurodegenerative disorders like AD. Finally, the inventors demonstrated that antiviral medication worked to diminish this AD-like phenotype, suggesting that this type of intervention might be useful for clinical treatment of AD.

The human brain-like tissue models of AD of the present invention offers several significant advantages over other available models of AD. First, the model disclosed herein encompasses many facets of human AD, including Aβ plaque formation, neuronal loss, reactive gliosis, neuroinflammation, and diminished neural network functionality. Importantly, the present model achieves this robust AD phenotype in a short time relative to other 3D model systems of AD [33]. Second, the model requires only unmodified, wild type cells as input, and it functions in the absence of any exogenous factors specifically known to regulate or induce AD [33-35]. Thus, this model of herpes-induced AD will enable future studies that aim to elucidate the mechanisms of HSV-1 mediated AD pathogenesis and to identify potential downstream targets for treating this devastating disease. See the section in the Examples titled “Discussion” for a more detailed discussion of previously developed AD models and the advantages of the present model.

### In Vitro Models of Alzheimer's Disease:

The present invention provides in vitro models of Alzheimer's disease (AD). The models comprise a population of human induced neural stem cells (hiNSCs) infected with a low multiplicity of infection of herpes simplex virus type 1 (HSV-1), where the population of hiNSCs in the AD model have an AD-phenotype.

As used herein, the term “in vitro model” is used to describe a disease model formed in an artificial environment (e.g., a test tube, reaction vessel, or cell culture) as opposed to a model comprising a multi-cellular organism. The in vitro models of the present invention are models of Alzheimer's disease (AD), meaning that they exhibit AD-associated phenotypes.

The in vitro models of AD described herein comprise hiNSCs that were infected with a low multiplicity of infection (MOI) of herpes simplex virus-1 (HSV-1). The inventors tested the ability of HSV-1 infection to generate a physiologically relevant human model of AD. They discovered that when hiNSCs are infected with a high MOI of HSV-1, few cells survive, as many undergo apoptosis. However, surprisingly, when hiNSCs were infected at a low MOI, e.g. an MOI of 0.0001, the cells undergo a profound morphological changes and upregulated AD mediators, consistent with an AD-like pathology (see Example 1). Thus, as used herein the term “low multiplicity of infection (MOI)” refers to an MOI of less than 0.001, preferably less than 0.0001.

The in vitro model comprises a population of HSV-1 infected hiNSCs that display AD-like phenotypes. Advantageously, the in vitro models of AD exhibit one or more phenotypes associated with AD. As used herein, “AD-associated phenotypes” refers to any pathological feature, characteristic, or biomarker of Alzheimer's disease seen within the cell population. Suitable AD-associated phenotypes for use with the present invention include, without limitation, features associated with amyloid plaques, neurofibrillary tangles, and inflammation.

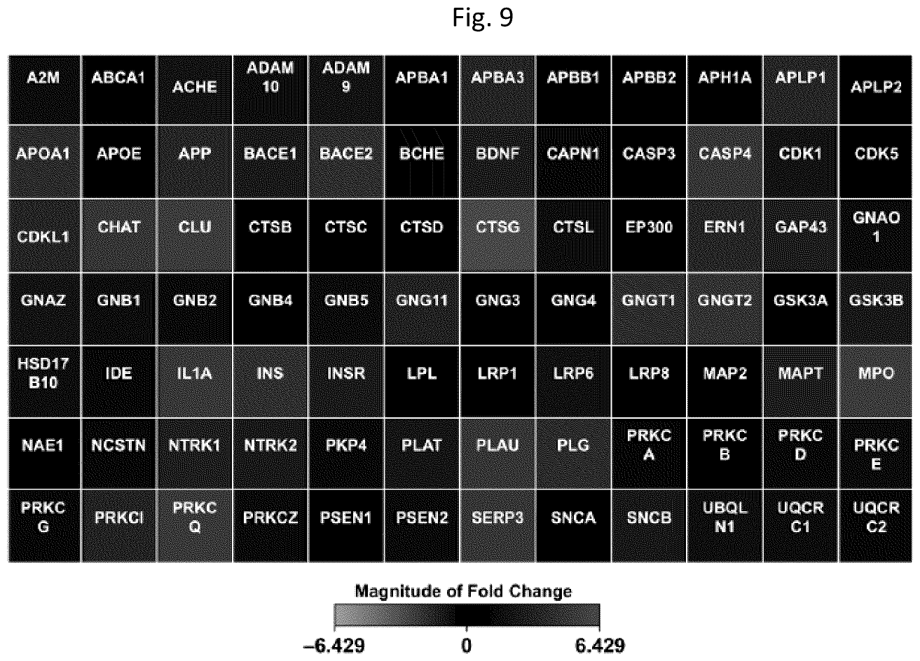

Thus, in some embodiments, the HSV-1 infected hiNSCs within the models exhibit (a) large, multicellular, dense Aβ+ fibrillar plaque-like formations (PLFs), (b) expression of PSEN1 and PSEN2 at higher levels as compared to non-infected control cells, (c) reactive gliosis, (d) one or more indicators of neuroinflammation, or (e) one or more of (a)-(d). Further, in some embodiments, the HSV-1 infected hiNSCs have a transcriptomic signature with increased or decreased level of relative expression levels of different genes as demonstrated in FIG. 9.

Aβ+ fibrillar plaque-like formations (PLFs) can be monitored visually or optically. As described in the examples, suitable methods to visually plaque size include, for example, fluorescent dye labeling of the HSV-1 infected hiNSCs, and monitoring and tracking size of amyloid plaques over time. One suitable dye is Thioflavin T dye, but other suitable dyes are known and understood in the art. One suitable tracking system include Incucyte Live-Cell analysis system (Sartorius) but other equivalent systems may also be used. Further, the genetically modified hiNSCs described herein that express one or more reporter gene may be used to monitor PLF formation without additional dyes or reagents.

Another characteristic of the AD-like phenotype, includes the enlargement, blebbing and fusion of nuclei to form these multicellular plaques. Suitably, this can be monitored by methods known in the art, including the use of a nuclear dye, preferably a fluorescent nuclear dye, and the real time monitoring and quantification of abnormal nuclei vs normal nuclei within the AD-model culture. The reduced number of healthy normal-sized nuclei would be indicative of abnormal AD-like phenotype.

Another AD-phenotype characteristic includes the increased expression of PSEN1 and PSEN2, which are associated with AD development. PSEN1 and PSEN2 overexpression is thought to play an important role in the generation of amyloid beta from amyloid precursor protein (APP), the accumulation of amyloid beta is associate with AD-phenotype. Suitable methods of detecting and monitoring expression of PSEN1 and PSEN2 within cells of the AD-model described herein are known and understood in the art, including, immunohistochemical staining, flow cytometry, PCR, qPCR, western blot, among others, and as demonstrated in the examples. Suitably, the AD-phenotype is characterized by an increase expression of PSEN1, PSEN2 or both when compared to hiNSCs not infected with the HSV virus (non-infected control cells).

The AD model described herein may also be monitored for the development of reactive gliosis. Gliosis is the reactive change of glial cells in response to damage to the central nervous system, and involves the proliferation or hypertrophy of several different types of glial cells, including, astrocytes, microglia and oligodendrocytes, resulting in more or larger glial cells causing scarring and dysfunction within the brain. Suitable methods of monitoring development of reactive gliosis include, for example, real time monitoring of glial cells within the 2-D culture. As described herein, the AD model may comprise hiNSCs that are genetically altered with a second reporter gene under the control of a promoter that only is expressed in glial cells (e.g., glial fibrillary acidic protein (GFAP) promoter) which can be used to monitor the glial cells in the model in real time.

Further, the AD-phenotype may include indicators of neuroinflammation. Suitably, in some embodiments, the indicator of neuroinflammation is the expression of one or more inflammatory markers. Suitable inflammatory markers are known and some are demonstrated in the Qiagen RT profiler for Human Alzheimer's Disease, presented in FIG. 9. For example, some suitable inflammatory markers include, for example, tumor necrosis factor alpha (TNFα), interleukin-1beta (IL1b), Serpina3, Lipocalin2, Interferon gamma (IFNg), and Interleukin-6 (IL-6). In another embodiment, the indicators of neuroinflammation include morphological changes in astrocytes, for example, a morphological change of one or more ‘star’ like, that can be used as imaging outcomes.

As used herein, the term “human induced neural stem cells (hiNSCs)” is used to describe a population of neural stem cells that was generated by reprogramming a human somatic cell. The somatic cell may be a donor cell from a subject or may be a cell from a subject having or suspected of having one or more symptoms of Alzheimer's disease. Suitable methods of generating hiNSC is described below in the section titled “Methods of generating an in vitro model of Alzheimer's disease,” These induced neural stem cells have the ability to give rise to the cells that make up the nervous system, including, e.g., neurons, glia, astrocytes, and oligodendrocytes. The presently described AD model uses these expandable and rapidly differentiating human induced neural stem cell (hiNSC) lines developed by the inventors. The hiNSCs are produced through the direct reprogramming of somatic starting cells such as dermal fibroblasts (see, e.g., D. M. Cairns, et. al, Expandable and rapidly differentiating human induced neural stem cell lines for multiple tissue engineering applications. Stem Cell Reports 7, 557-570 (2016) [28], and U.S. Pat. No. 10,662,409, the contents of which are incorporated by reference in their entireties. These hiNSC lines are ideally suited for this brain tissue model for a variety of reasons. First, the hiNSCs spontaneously and rapidly differentiate into multiple neuronal and glial subtypes that are stable in long-term culture. Advantageously, this differentiation protocol does not require specialized media, and can be completed in as few as four days. Second, the inventors have demonstrated that the hiNSCs are highly responsive to various growth factors, guidance cues, and pharmaceuticals; and that they are highly infectable by neurotropic viruses such as Zika[29]. Third, the inventors have demonstrated that the hiNSCs grow well in their previously established 3D brain model[30]. The combination of this model and HSV infection allow for the development of this new in vitro model of sporadic AD. The model can be either 2D in tissue culture dish or a 3D brain-like tissue model involving seeding the HSV-infected cells into a biomaterial-based scaffold in order to recapitulate structural features representative of native brain tissue. Porous silk protein sponges were formed into concentrically shaped donuts recapitulating the neuron-rich gray matter of the brain, which were subsequently infused with type I collagen gel to support the growth of neurite extensions, thereby mimicking the axon-dense white matter. This brain tissue model system allows for visualization and quantification of neurite patterning and networks as well as electrophysiological readouts in real time.

In another aspect, the in vitro models of AD disclosed herein are used for both high throughput screening and personalized medicine applications. For personalized applications, somatic cells (e.g., dermal fibroblasts) are harvested from patients and reprogrammed into hiNSCs. Using patient-specific hiNSCs will allow for the identification of drugs that are best suited for treating an individual patient's form of AD. Thus, in some embodiments, the in vitro models of AD comprise hiNSCs were reprogrammed from somatic cells from a specific subject. In preferred embodiments, the somatic cells are from a patient diagnosed with AD or at risk of developing AD.

Loss of neural tissue functionality is a hallmark of AD. To facilitate studies of neuronal function in in vitro models of AD, the inventors have generated genetically modified hiNSCs comprising reporter genes that are only expressed if the hiNSCs differentiate into mature, functioning neurons. In Example 2, the inventors describe modified hiNSCs that were generated via stable transduction with a construct (i.e., a lentiviral construct) that encodes a reporter gene under the control of neuron specific reporter. Any neuron specific promoter may be used in these reporter constructs including, without limitation, a synapsin promoter or a CaMKIIa promoter.

In some embodiments, the hiNSCs used in the in vitro models are genetically modified to stably express a reporter gene under the control of a synapsin promoter, allowing for neuron specific expression of the reporter gene. Suitable synapsin promoters or fragments thereof are known in the art and include, for example, the human synapsin I promoter (SEQ ID NO:1).

As used herein, the term “reporter gene” is used to refer to a gene encoding a protein that is readily quantifiable or observable. While the expression of any gene can be quantified (e.g., using polymerase chain reaction, northern blotting, western blotting, fluorescent in situ hybridization, etc.), commonly used reporter genes include those that can be identified visually, e.g., using a colorimetric, fluorescent, or luminescent signal. Exemplary reporter systems include, for example, genes encoding β-galactosidase, chloramphenicol acetyltransferase, luciferase, fluorescent proteins, such as, for example, green fluorescent protein (GFP), and mcherry. In some embodiments, the reporter gene used with the present invention is a fluorescently tagged calcium sensor. Calcium sensors allow one measure neural activity within the in vitro model via calcium imaging. Calcium sensors include genetically encoded Ca²⁺, indicators (GECIs), which can be used for visualizing calcium at the levels of cell populations, single cells or subcellular compartments. GCaMP is one example of a GECI, which was developed by Junichi Nakai (Nature Biotechnol. 19:137-141, 2001). GCaMP is a fusion protein comprising circularly permutated green fluorescent protein (GFP), calmodulin (CaM; a symmetrical, hinge-like protein that binds to four calcium ions) and M13, a calmodulin-binding peptide from myosin light chain kinase. In the absence of calcium, the circularly permutated GFP exists in a poorly fluorescent state. However, when calcium is present, CaM undergoes a conformational change that allows it to bind to M13, thereby bringing the N- and C-terminal ends of GFP in close proximity and producing a fluorescent signal. This original GCaMP molecule has been extensively modified and improved. Any such GECI can be used with the present invention including, for example, GCaMP, GCamP3, GCamP2, GCaMP4, GCaMP5, GCaMP6, GCaMP7, GCaMP-X, and modifications thereof, including those described by Dana et al. (Nat Methods 16(7):649-657, 2019) and Yang et al. (Nat Commun 17; 9(1):1504, 2018). Additionally, red fluorescence GECIs, termed “RCaMPs” may be used with the present invention including, for example, the GECI “srGECO” provided as SEQ ID NO:2.

Additionally, the genetically modified hiNSCs of the present invention may comprise an optogenetic sensor. In the Examples, the inventors generated hiNSCs that encode a channelrhodopsin protein (see Example 2). Channelrhodopsins are light-gated ion channels that serve as sensory photoreceptors in unicellular green algae. The channelrhodopsin component allows for specific optical stimulation of only those cells expressing channelrhodopsin. For example, expression of channelrhodopsin in specific cells can be used to stimulate only certain neurons to understand the downstream effects on the rest of the brain. Thus, use of such optogenetic sensors can be used to focally stimulate specific areas of the in vitro models of AD described herein.

Reactive gliosis is another hallmark of AD, and the HSV-1-infected hiNSCs of the present invention exhibit robust gliosis. To facilitate the detection of gliosis in in vitro models of AD, the hiNSCs used in the models may be genetically modified to comprise reporter genes that are only expressed if the hiNSCs differentiate into glia. Thus, in some embodiments, the hiNSCs are genetically modified to further express a second reporter gene under the control of a glial fibrillary acidic protein (GFAP) promoter (i.e., a glia specific reporter), wherein the expression of the second reporter gene is indicative of gliosis. Suitable GFAP promoters include, for example, the GFAP promoter of SEQ ID NO:3. The second reporter gene used with the hiNSCs may be any reporter gene. Suitable constructs comprising a GFAP promoter are commercially available, e.g., the adenoviral construct pAAV-GFAP-hChR2(H134R)-mCherry available from Addgene.

Alternatively, the hiNSCs used in the models may be genetically modified to express a reporter gene under the control of the tumor necrosis factor (TNF)-α promoter, such as the one described by www.invivogen.com/hek-blue-tnfa, which provides a means to monitor the activation of an inflammatory response (i.e., the NF-κB pathway) as a proxy for neuroinflammation. Alternatively, the hiNSCs may be genetically modified to express a reporter gene under the control of the presenilin-2 (PSEN2) promoter (see Brain Res Mol Brain Res 56(1-2):57-65, 1998). PSEN2 is a gene that is commonly mutated in inherited forms of AD.

The inventors have demonstrated that the in vitro models of AD disclosed herein can be used in at least two distinct formats. In a first format, the hiNSCs are grown in a monolayer culture (2D), i.e., a culture in which cells are grown in a single layer on a surface (e.g., the bottom of a flask or Petri dish). The simplicity of this format offers advantages for high-throughput applications. For example, monolayer culture is more amenable to real-time assessments, e.g., using the Incucyte imaging and analysis equipment and associated software. Monolayer culture-based models are also faster to generate, achieving AD-like phenotypes in approximately one week.

In a second format, the hiNSCs are grown in a three-dimensional biomaterial-based scaffold. For example, the inventors have incorporated these cells into a 3D model using silk-based sponge scaffolds infused with collagen gels (see Example 2). This established 3D brain-like tissue model has the advantage of more accurately modeling the native brain tissue environment. The compartmentalization of these models provides more standardized and predictable features to probe. Additionally, the 3D brain-like tissue model allows for longer-term culture, which is advantageous for studying a slow developing AD phenotype or the chronic impact of additives, drugs, or nutrients on disease outcomes. As compared to the current state of the art technologies, the 3D brain-like models of the present invention can achieve more robust phenotypes in a shorter period of time. The 3D models of the present invention also offer the use of a centralized window for visualizing and quantifying neural activity in real-time.

The in vitro models of AD disclosed herein are designed to be used for several applications, including high-throughput screens for compounds that alter an AD-associated phenotype. A “high throughput screen,” as used herein, refers to an assay that tests many compounds simultaneously for their effects on a trait of interest. High throughput screens may be performed using any equipment that facilitates mass testing including, for example, multiwell plates, automated liquid handling devices, microarrays, and microfluidic systems.

In some embodiments, the high-throughput screen would look for the reduction in the formation or size of Aβ+ fibrillar plaque-like formations (PLFs) within the AD-model. In other embodiments, it is the reduction in the expression of PSEN1 and PSEN2, and or reduction in one or more indicator of inflammation within the AD model when exposed to the compound being screened.

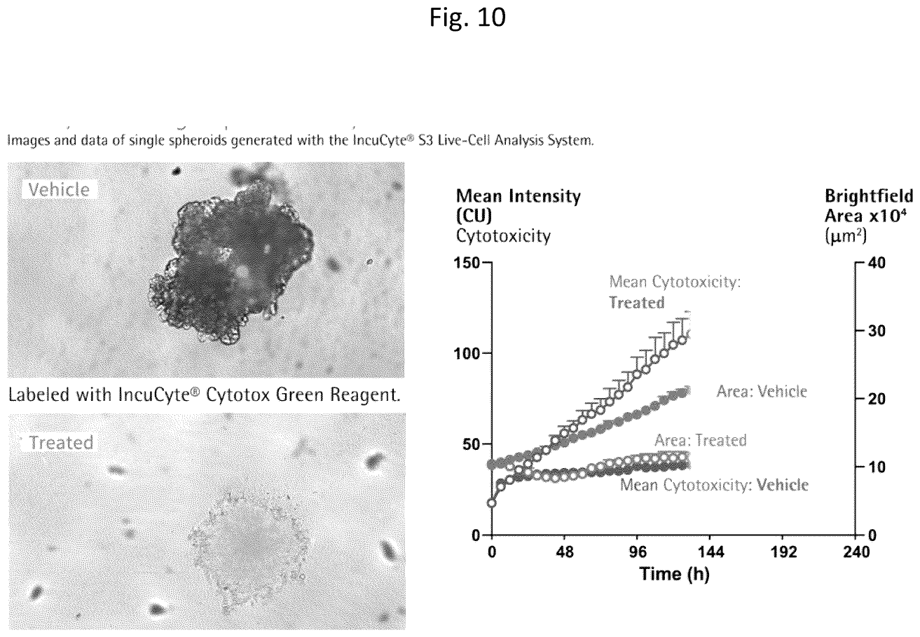

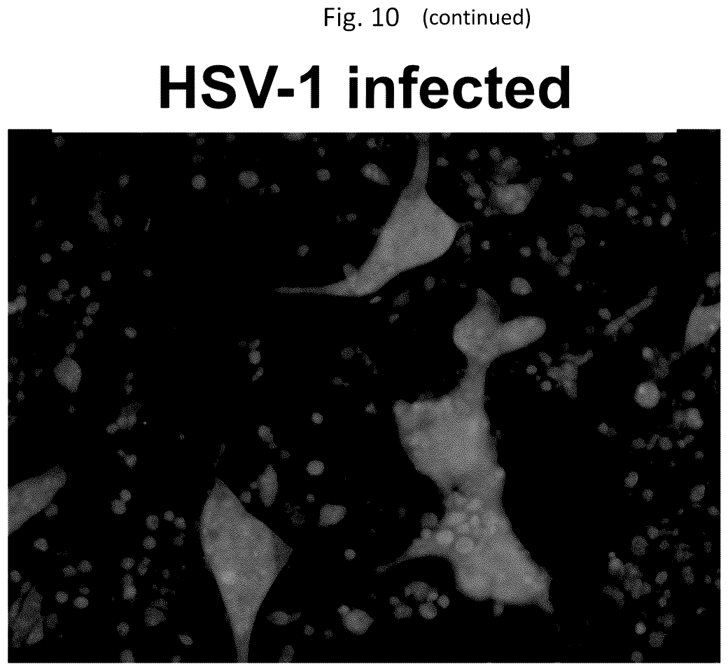

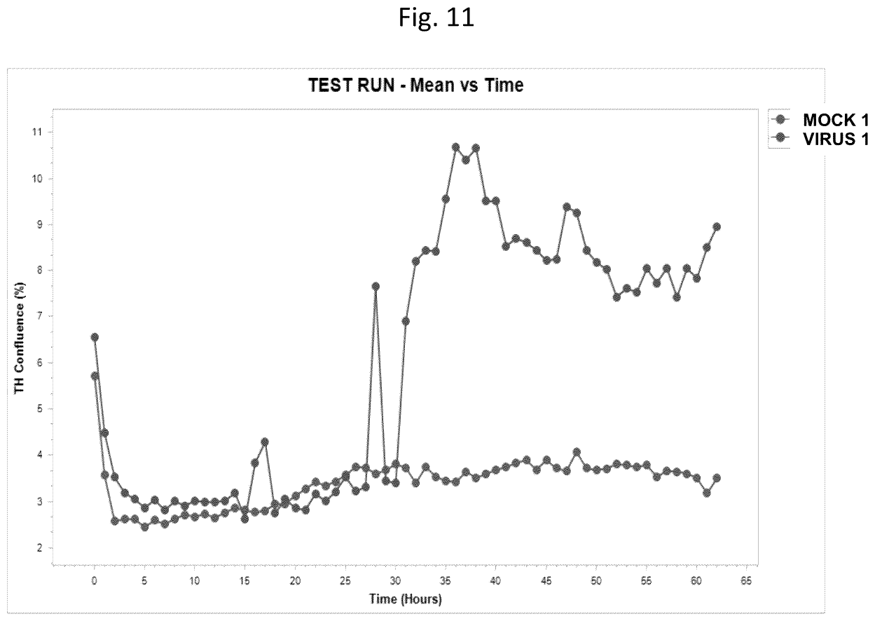

In some embodiments, the AD-associated phenotype is the presence, formation, or growth of Aβ+ fibrillar plaques. Detection of amyloid plaques can be accomplished, for example using Thioflavin T dye. The inventors have demonstrated that HSV-1-infected hiNSCs display large plaque-like formations that stain positive with Thioflavin T dye (see FIG. 10), and that the signal produced by this dye can be monitored over time to track the development or treatment of amyloid plaques (see FIG. 11).

In other embodiments, the AD-associated phenotype is the enlargement, blebbing, and fusion of nuclei. The state of the nuclei in the hiNSCs can be assessed, for example, using nuclei-specific stains (e.g., DAPI) or fluorescently tagged nuclear proteins. For example, the hiNSCs may be stably infected with Incucyte® NucLight Red Lentivirus, which expresses a generic red fluorescent protein that is restricted to the nucleus. This allows for real-time monitoring of nuclear shape and size, and ultimately for quantification of “healthy” nuclei (i.e., nuclei of a predetermined, uniform size and shape).

In some embodiments, the in vitro model of AD is produced by the methods disclosed herein. See section titled “Methods of generating an in vitro model of Alzheimer's disease” below.

### Genetically Modified Human Induced Neural Stem Cells:

The present invention provides genetically modified human induced neural stem cell (hiNSC). The hiNSC comprise a construct (including, for example a vector, more particularly a viral vector) encoding at least one reporter gene under the control of a synapsin promoter. Because this promoter is neuron-specific, expression of the one or more reporter genes indicates that the hiNSC has differentiated into a mature, functioning neuron.

As used herein, the term “construct” refers to a polynucleotide sequence comprising a promoter operably connected to a polynucleotide sequence encoding the one or more reporter genes described herein which can be expressed in a cell. In some embodiments, the polynucleotide construct is a vector. As used herein, the term “vector” or “expression vector” refers to a nucleic acid molecule capable of propagating another nucleic acid to which it is linked and capable of directing the expression of nucleic acids to which they are operatively linked. The term includes the vector as a self-replicating nucleic acid structure as well as the vector incorporated into the genome of a host cell into which it has been introduced. The term vector encompasses “plasmids”, the most commonly used form of vector. Plasmids are circular double-stranded DNA loops into which additional DNA segments (e.g., those encoding one or more peptides) may be ligated. Preferably, the vector is a viral vector (e.g., replication defective retroviruses, adenoviruses and adeno-associated viruses. The vectors may also comprise appropriate control sequences that allow for translational regulation in a host cell. In some embodiments, the vectors further comprise nucleic acid sequences encoding one or more agents or tags. In some embodiments, the vectors further comprise additional regulatory sequences, such as signal sequences. In some embodiments, the vectors of the present invention further comprise heterologous backbone sequence. As used herein, “heterologous nucleic acid sequence” refers to a non-human nucleic acid sequence, for example, a bacterial, viral, or other non-human nucleic acid sequence that is not naturally found in a human. Heterologous backbone sequences may be necessary for propagation of the vector and/or expression of the encoded peptide.

Viral vector can be packaged into a viral particle or virus, and the term may refer to the polynucleotide and the virus encapsulating such polynucleotide sequence. A virus comprising the viral vector can be used to transfer the viral vector into the hiNSCs, through a process known as transduction. Suitable viral vectors are known in the art and include, but are not limited to, an adenovirus vector; an adeno-associated virus vector; a pox virus vector, such as a fowlpox virus vector; an alpha virus vector; a baculoviral vector; a herpes virus vector; a retrovirus vector, such as a lentivirus vector; a Modified Vaccinia virus Ankara vector; a Ross River virus vector; a Sindbis virus vector; a Semliki Forest virus vector; and a Venezuelan Equine Encephalitis virus vector. In a preferred embodiment, the viral vector is a lentiviral vector. Suitable vectors are known and available to one skilled in the art for the practice of this invention.

### Methods of Generating an In Vitro Model of Alzheimer's Disease:

The present invention provides methods of generating an in vitro model of Alzheimer's disease. The methods comprise: (a) infecting a population of human induced neuronal stem cells (hiNSCs) with a low multiplicity of infection of herpes simplex virus-1 (HSV-1) to produce a population of herpes-infected hiNSCs, and (b) culturing the herpes-infected hiNSCs in culture medium for at least three days. Importantly, these methods cause the hiNSCs develop an AD-like phenotype.

To generate the herpes-infected hiNSCs used with the present invention, the hiNSCs are infected with a low multiplicity of infection (MOI) of herpes simplex virus-1 (HSV-1). In some embodiments of the present methods, the hiNSCs are contacted with the HSC-1 at a multiplicity of infection of 0.001 or less, preferably 0.0001 or less.

The cells are cultured for at least three days after HSV infection, and can be cultured for longer time period, e.g., at least four days, at least five days, at least six days, at least seven days, at least eight days, at least nine days, at least ten days, etc. Notably, the time required for the cells to develop an AD-like phenotype will vary slightly depending of the timing of infection. For example, more mature cells can withstand the effects of HSV-1 infection for a longer time period (around one week). Alternatively, the hiNSCs may be infected for only 24-48 hours to kick start the neuroinflammatory cascade. The cells are then treated with valacyclovir to halt ongoing infection and cultured in normal culture media as a way to assess longer term downstream effects of recovered virus infection.

As discussed above, the inventors have demonstrated that the herpes-infected hiNSCs described herein can be incorporated into a three-dimensional biomaterial-based scaffold to serve as a three-dimensional human brain model of herpes-induced AD. Thus, in some embodiments, the methods further comprise seeding hiNSCs into a biomaterial-based scaffold after step (a). In specific embodiments, the biomaterial-based scaffold comprises a silk-based sponge scaffold infused with collagen gel.

In some embodiments, the methods are used to generate an in vitro model of AD comprising hiNSCs that were reprogrammed from somatic cells from a subject. Such models may be used, for example, in personalized medicine applications, as discussed above.

In some embodiments, the methods are to generate an in vitro model of AD comprising genetically modified hiNSCs. As discussed above, models that comprise genetically modified hiNSCs can be used to readily assess neuron function without additional components necessary. Thus, in some embodiments, prior to step (a), the methods further comprise genetically modifying the hiNSCs by contacting the cells with a construct comprising one or more reporter genes under the control of a synapsin promoter. In this system, the expression of the one or more reporter genes indicates that hiNSCs have differentiated into mature, functioning neurons given the promoter only allows expression of the reporter gene in neuron cells.

In some embodiments, the construct used to genetically modify the hiNSCs is a viral vector. In preferred embodiments, the resulting genetically modified hiNSCs stably express the one or more reporter genes. Stable expression is achieved when the construct comprising the one of more reporter genes is successfully integrated in the cellular genome, allowing the cells to pass on the reporter gene(s) to future generations. In some embodiments, the one or more reporter genes comprise (i) channelrhodopsin, (ii) a fluorescently tagged calcium sensor or (iii) both. Suitable calcium sensor are described herein for use in the methods described.

In some embodiments, the methods comprise genetically modifying the hiNSCs to express a second reporter gene under the control of a glial fibrillary acidic protein (GFAP) promoter. As is discussed above, the expression of a GFAP-driven reporter gene is indicative of gliosis.

The hiNSCs used with the present invention are generated by the direct reprogramming of human somatic cells, as previously described in Cairns et al. (Stem Cell Reports, 2016. 7(3):557-70) and in U.S. Pat. No. 10,662,409, which are both incorporated by reference herein in their entirety. For a brief description of this reprogramming method, see the Materials and Methods section of Example 1. Importantly, the hiNSCs produced by this method can be differentiated into neurons and/or glia without the need to form an intermediate neurosphere, which is cumbersome and is commonly required in previous methods. For the purposes of tissue engineering, avoiding neurosphere formation is ideal because once cells have been equilibrated to low adhesion conditions, the transition to adherent monolayer or 3D culture is sometimes difficult.

Thus, in some embodiments, the hiNSCs used in the methods are generated by: (a) providing one or more human somatic cells; (b) inducing transient expression of OCT4, KLF4, SOX2, and cMYC in the somatic cells for 2-6 days forming reprogrammed somatic cells; (c) providing a plurality of inactivated embryonic fibroblasts; and (d) contacting the reprogrammed somatic cells with the inactivated embryonic fibroblasts in a culture media comprising 20% KO DMEM xenogen-free serum replacement and at least 15 ng/ml recombinant bFGF to generate hiNSCs.

A “somatic cell” is any cell of a multicellular organism other than a gamete, germ cell, gametocyte, or undifferentiated stem cell. Examples of somatic cells include, but are not limited to fibroblasts, adipocytes, dermal cells, epidermal cells, muscle cells, and bone cells. In previous work, the inventors made the surprising discovery that hiNSCs may be created from any human somatic cell, not only those of ectodermal origin (see U.S. Pat. No. 10,662,409). Thus, any human somatic cell may be used with the methods of the present invention.

Any known method can be used to induce transient expression of OCT4, KLF4, SOX2, and cMYC in the somatic cells. Exemplary methods are described, for example, in Malik and Rao 2013 (Methods Mol Biol. 997:23-33), which is hereby incorporated by reference in its entirety. In some embodiments, transient expression is accomplished using a vector (e.g., a viral vector).

The present methods utilize inactivated embryonic fibroblasts as feeder cells (i.e., cells that provide extracellular secretions to help another cell to proliferate). As used herein, the term “inactivated fibroblast” refers to a mitotically inactivated fibroblast. Methods of preparing inactivated fibroblasts are well known in the art and include, for example, methods that utilize a DNA crosslinking agent (e.g., mitomycin C) or irradiation (e.g., gamma irradiation) to inactivate the cells. Exemplary methods of preparing inactivated fibroblasts are described in Conner 2001 (Current Protocols in Molecular Biology. 51:23.2:23.2.1-23.2.7), which is hereby incorporated by reference in its entirety. In some embodiments, inactivated human embryonic fibroblasts are used. In some embodiments, inactivated mouse embryonic fibroblasts are used.

The present methods utilize xenogen-free media (i.e., media containing no non-human substances) to maintain the hiNSCs in an undifferentiated state. The xenogen-free media used herein comprises 20% xenogen-free KO Dulbecco's Modified Eagle Medium (DMEM) serum replacement and at least 15 ng/ml recombinant basic fibroblast growth factor (bFGF).

In some embodiments, the hiNSCs are encouraged to differentiate, in whole or in part, through removal from feeder cells and exposure to a low bFGF environment. In other embodiments, the hiNSCs are encouraged to differentiate into specific neuronal subtypes through addition of fibroblast growth factor 8 (FGF8), retinoic acid (RA), sonic hedgehog (Shh), or a combination thereof to the culture media (see U.S. Pat. No. 10,662,409).

Methods of Screening for Compounds that Alter an Alzheimer's Disease-Associated Phenotype:

The present invention provides methods of screening for compounds that alter an AD-associated phenotype. The methods comprise (a) contacting an in vitro model of AD described herein with a compound; and (b) detecting a change in one or more AD-associated phenotype in the in vitro model of AD.

Exemplary compounds include, but are not limited to, for example, small molecules, drugs, peptides, proteins, antibodies, nutraceuticals, nutrients, foods, alcohol, caffeine, nicotine and other factors to which we are exposed to in foods or the environment. One skilled in the art would be able to determine suitable compounds and compositions to be tested using the model described herein.

As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not a protein or polypeptide (e.g., is not an oligopeptide or peptide). In some embodiments, a small molecule is not a polynucleotide (e.g., is not an oligonucleotide). In some embodiments, a small molecule is not a polysaccharide. In some embodiments, a small molecule does not comprise a polysaccharide (e.g., is not a glycoprotein, proteoglycan, glycolipid, etc.). In some embodiments, a small molecule is not a lipid. In some embodiments, a small molecule is a modulating agent. In some embodiments, a small molecule is biologically active. In some embodiments, a small molecule is detectable (e.g., comprises at least one detectable moiety). In some embodiments, a small molecule is a therapeutic. Those of ordinary skill in the art, reading the present disclosure, will appreciate that certain small molecule compounds described herein may be provided and/or utilized in any of a variety of forms such as, for example, salt forms, protected forms, pro-drug forms, ester forms, isomeric forms (e.g., optical and/or structural isomers), isotopic forms, etc.

In some embodiments, the change in one or more AD-associated phenotype includes a change in: (a) the number of Aβ+ fibrillar plaque-like formations (PLFs); (b) expression of PSEN1 and PSEN2; (c) neuronal cell loss; (d) one or more indicators of neuroinflammation; (e) neural network functionality; or (f) one or more of (a)-(e). Methods of detecting and/or monitoring these changes are described herein and are understood by one skilled in the art.

In some embodiments, the in vitro model of AD comprises genetically modified hiNSCs and the method comprises visualizing neuronal growth and function within the in vitro model of AD, preferably without addition dyes or reagents. Suitable genetically modified hiNSCs are described in more detail above and in the examples can be used to monitor the AD-phenotype over time without additional dyes or reagents. The cells comprising the reporter gene can be used to monitor cell functions in real time high throughput optical screening, including, for example, ability to assess neuron stimulation, to assess changes in healthy vs. AD-neurons, to assess changes in neuroinflammation or other outcomes associated neurodegenerative disease phenotypes.

For example, as described in the Examples, the use in the model of lentiviral transduced hiNSCs expressing channelrhodopsin and a cherry-red calcium sensor under the control of a synapsin promoter allows for gene expression only in neurons that are fully differentiated and functioning properly. Given that this calcium sensor is driven by a synapsin promoter, any cherry red expression indicates proper functioning of mature neurons. Channelrhodopsin could also be used to image firing post-optostimulation in healthy vs. AD neurons. Additionally, genetically modifying the cells to comprise a puromycin cassette driven by a constitutively active CMV promoter allows for stable selection of cells with this construct at the proliferative hiNSC stage.

In some embodiments, the method identifies a compound that exacerbates an AD-associated phenotype. For example, in one embodiment, the identified compound increases the rate of development and/or increases the size of Aβ+ fibrillar plaque-like formations (PLFs) in vitro model of AD. Increases in the rate of development and/or increased size of PLFs can be determined, e.g., optically or visually (e.g., immunostaining, real-time fluorescent cellular analysis (live-cell system or the like using fluorescent proteins).

In other embodiments, the method identifies a compound that reduces an AD-associated phenotype. Reducing in AD-associated phenotype would be a reduction in one or more characteristic associated with the phenotype, e.g., reduction in number, rate of development or size of Aβ+ fibrillar plaque-like formations (PLFs), reduction in neuronal cell loss, increase in neuronal cell growth, and reduction in one or more indicators of neuroinflammation. A reduction if neuroinflammation may be detected, for example, by monitoring the expression of a reporter gene under the control of the TNF locus. These compounds may undergo further screening for the ability to be used in the treatment of a subject having AD. Suitably, the compounds may be made by methods of manufacture for producing compounds suitable for administration to a human.

In preferred embodiments, the AD-associated phenotype that exhibits a change (e.g., a decrease) in the presence of an identified compound is a phenotype associated with sporadic AD. The compounds identified may undergo further testing and development for use in treating or inhibiting symptoms of AD, including sporadic AD.

### Methods of Assessing Alzheimer's Disease-Associated Phenotypes in Real Time:

The present invention provides methods of tracking and quantifying the development of AD-associated plaques in real time. The methods comprise imaging live cells within an in vitro model of AD described herein over time.

The use of the genetically altered hiNSCs described herein having a fluorescent readout allows for the real time monitoring of the development of AD plaques and other physiological changes to the neuronal cells over time or when in contact with test compounds.

Additionally, the present invention provides methods of tracking and quantifying post-stimulation firing of neurons in real time. The methods comprise (a) stimulating the cells within an in vitro model of AD described herein with light, and (b) measuring the firing of neurons within the in vitro model of AD over time.

The present disclosure further provides kits for carrying out the methods and assays described herein. In one embodiment, the kit comprises an in vitro AD model described herein and methods of use. In another embodiment, the kit comprising an in vitro AD model comprising the genetically altered HSV infected hiNSCs with an AD-phenotype described herein.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of” and “consisting of” those elements. The term “consisting essentially of” and “consisting of” should be interpreted in line with the MPEP and relevant Federal Circuit interpretation. The transitional phrase “consisting essentially of” limits the scope of a claim to the specified materials or steps “and those that do not materially affect the basic and novel characteristic(s)” of the claimed invention. “Consisting of” is a closed term that excludes any element, step or ingredient not specified in the claim. For example, with regard to sequences “consisting of” refers to the sequence listed in the SEQ ID NO. and does refer to larger sequences that may contain the SEQ ID as a portion thereof.

All publications, patent applications, patents, and other references mentioned herein are incorporated by reference in their entirety. In the case of conflict, the present specification, including definitions, will control.

Other features and advantages of the invention will be apparent from the description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting.

### EXAMPLES

### Example 1: Generation of a 3D Human Brain-Like Tissue Model of Ad

Alzheimer's disease (AD) is a neurodegenerative disorder that causes cognitive decline, memory loss, and inability to perform everyday functions. Hallmark features of AD-including generation of amyloid plaques, neurofibrillary tangles, gliosis, and inflammation in the brain—are well defined; however, the cause of the disease remains elusive. Growing evidence implicates pathogens in AD development, with herpes simplex virus type I (HSV-1) gaining increasing attention as a potential causative agent. In the following Example, the inventors describe a multidisciplinary approach to produce physiologically relevant human tissues to study AD using human-induced neural stem cells (hiNSCs) and HSV-1 infection in a 3D bioengineered brain model. They report a herpes-induced tissue model of AD that mimics human disease with multicellular amyloid plaque-like formations, gliosis, neuroinflammation, and decreased functionality, completely in the absence of any exogenous mediators of AD. In future studies, this model will be used to identify potential downstream drug targets for treating this devastating disease.

### Materials and Methods:

Generation of hiNSCs

hiNSCs were generated as previously described (28). Briefly, human foreskin fibroblasts were plated at a concentration of 10⁵ cells in one gelatin-coated well of a six-well plate and cultured in fibroblast media [Dulbecco's modified Eagle's medium (DMEM), 10% fetal bovine serum, and 1% antibiotic-antimycotic]. Concentrated aliquots of a polycistronic lentivirus expressing OCT4, KLF4, SOX2, and cMYC (Addgene, no. 24603, a gift from J. Cibelli) were used to infect cells in fibroblast medium using polybrene (Millipore) at a MOI of 1 to 2. Media was changed to hiNSC media: knockout (KO) DMEM supplemented with 20% KO xeno-free serum replacement, recombinant basic fibroblast growth factor (20 ng/ml), 1% glutamax, 1% antibiotic-antimycotic, and 0.1 mM P-mercaptoethanol, which also contained 1% KO growth factor cocktail (GFC) (Invitrogen). Four days later, cells were trypsinized and replated onto mouse embryonic fibroblast (MEF) feeder layers that had been previously inactivated by mitomycin C. hiNSC media (without KO-GFC) was subsequently changed every 1 to 3 days. At day 30 or later, colonies were mechanically picked and passaged onto fresh feeder MEF plates. Each picked colony was expanded to generate one hiNSC line. hiNSCs were enzymatically passaged as colonies using trypsin-like enzyme TrypLE (Invitrogen), expanded, and subsequently frozen to make stocks. All generated lines tested negative for mycoplasma contamination.

### HSV-1 Production and Titration

HSV-1 McIntyre strain VR-539 was purchased from American Type Culture Collection (Manassas, VA). Monolayers of Vero cells were infected with HSV-1 at a MOI of 0.01. After 48 hours in culture, infected cells were harvested with three freeze-and-thaw cycles to disrupt cell membranes and release virions. Cells were subjected to low-speed centrifugation, and virus titers were subsequently measured by standard plaque assay. The resulting titer of concentrated HSV-1 was 2×10⁷ plaque-forming units (PFU)/ml.

hiNSC Differentiation, HSV-1 Infection, and Valacyclovir HCl Treatment

hiNSC colonies were trypsinized off of MEF feeder layers using TrypLE (Invitrogen) and then dissociated by manual pipetting. Cell suspensions were passaged through a 40- to 70-μM cell strainer to remove larger aggregates. Dissociated hiNSCs were cultured on gelatin-coated plates in neurobasal media supplemented with 2% B27 (Invitrogen), 1% glutamax, and 1% antibiotic-antimycotic. For HSV infection, we used purified HSV-1 to directly infect hiNSCs at a range of MOIs (0.00001 to 1), which was calculated based on the starting virus concentration (2×10⁷ PFU/ml) and initial seeding density.

Once added to media, inoculum was allowed to remain in cell cultures for the duration of the experiment to mimic low-level brain infection. For mock infections, an equal volume of control culture medium from uninfected Vero cells was used. All virus work was approved by the Tufts Institutional Biosafety Committee. Valacyclovir (VCV) HCl was reconstituted in dH₂O and used at a concentration of 200 μM for all subsequent experiments. VCV was added at day 0 (D0; concurrently with HSV-1 infection) or day 1 (D1; 24 hours after infection).

### 3D Cortical Brain Tissue Model

A 3D cortical brain tissue model was generated as previously described (28, 30). Briefly, silk sponges with pore sizes of 500 to 600 m were prepared from 6% (w/v) Bombyx mori-derived silk solution. Sponges were biopsy-punched into 6-mm discs, with 2-mm holes punched in the center to form donut-shaped scaffolds. Scaffolds were autoclaved and coated with laminin (0.5 mg/ml; Roche, Indianapolis, IN). Dissociated hiNSCs were seeded into the silk porous scaffolds at a density of 10⁶ and allowed to adhere overnight. The following day, collagen gels were made using type I rat tail collagen (Corning, Bedford, MA, USA) as previously described. 3D human brain-like tissue constructs were then cultured in neurobasal media (Invitrogen, Carlsbad, CA) supplemented with 2% B27 (Invitrogen, Carlsbad, CA), 0.5 mM glutamax, and 1% antibiotic-antimycotic (Invitrogen, Carlsbad, CA) for 4 weeks to allow for mature network formation before subsequent HSV-1 infection, with media changes every 3 days.

### Quantitative Reverse Transcription PCR

Total RNA was isolated using the RNeasy Mini Kit (Qiagen). Complementary DNA was generated using iScript (Bio-Rad) according to the manufacturer's protocols. Quantitative reverse transcription PCR (qRT-PCR) was performed using SYBR Green and the CFX96 Real-Time PCR Detection System (Bio-Rad) and normalized against the housekeeping gene glyceraldehyde phosphate dehydrogenase. All primer sequences are listed in Table 1. The qPCR-based Qiagen RT Profiler Array for AD (catalog no. PAHS-057Z) was performed according to the manufacturer's instructions.

TABLE 1
qRT-PCR primer sequences.

| GENE | ACCESSION # | SEQUENCE 1 (5′-->3′) | SEQUENCE 2 (5′-->3′) |
|---|---|---|---|
| Amyloid precursor | NM_000484 | GTCTCTCTCCCTGCTCTACAA | GGCCAAGACGTCATCTGAATAG |
| protein (APP) | | (SEQ ID NO: 4) | (SEQ ID NO: 5) |
| Beta secretase 1 | NM_012104 | CCATCCTTCCGCAGCAATA | CGTAGAAGCCCTCCATGATAAC |
| (BACE1) | | SEQ ID NO: 6) | (SEQ ID NO: 7) |
| Tumor necrosis | NM_000594.4 | GAGGCCAAGCCCTGGTATG | CGGGCCGATTGATCTCAGC |
| factor alpha | | (SEQ ID NO: 8) | (SEQ ID NO: 9) |
| (TNF) | | | |
| Serpina3n (SERP3) | NM_001085.4 | GCTCATCAACGACTACGTGA | ACACCATTACCCACTTTTTCTTGC |
| | | (SEQ ID NO: 10) | (SEQ ID NO: 11) |
| Lipocalin 2 | NM_005564.5 | GACAACCAATTCCAGGGGAA | GCATACATCTTTTGCGGGTCT |
| (LCN2) | | G (SEQ ID NO: 12) | (SEQ ID NO: 13) |
| Presenilin 1 | NM_000021 | TGGCTACCATTAAGTCAGTCA | CCCACAGTCTCGGTATCTTCT |
| (PSEN1) | | GC (SEQ ID NO: 14) | (SEQ ID NO: 15) |
| Presenilin 2 | NM_012486 | CTGACCGCTATGTCTGTAGT | CTTCGCTCCGTATTTGAGGGT |
| (PSEN2) | | GG (SEQ ID NO: 16) | (SEQ ID NO: 17) |
| Interleukin 6 | NM_012486 | TCAATATTAGAGTCTCAACCC | TTGTTTTCTGCCAGTGCCTC |
| (IL-6) | | CC (SEQ ID NO: 18) | (SEQ ID NO: 19) |
| Interleukin 1 | NM_000576.2 | CAGAAGTACCTGAGCTCGCC | AGATTCGTAGCTGGATGCCG |
| beta (IL1B) | | (SEQ ID NO: 20) | (SEQ ID NO: 21) |
| Interferon gamma | NM_000619 | ACTGTCGCCAGCAGCTAAAA | TATTGCAGGCAGGACAACCA |
| (IFNy) | | (SEQ ID NO: 22) | (SEQ ID NO: 23) |
| Vimentin (VIM) | NM_003380.5 | TCCGCACATTCGAGCAAAGA | TGATTCAAGTCTCAGCGGGC |
| | | (SEQ ID NO: 24) | (SEQ ID NO: 25) |
| Glyceraldehyde | NM_002046.5 | ATTGCCCTCAACGACCACT | ATGAGGTCCACCACCCTGT |
| 3-phosphate | | (SEQ ID NO: 26) | (SEQ ID NO: 27) |
| dehydrogenase | | | |
| (GAPDH) | | | |
| Glial fibrillary | NM_001131019.2 | ACTGGCAGAGCTTGTTAGTG | AGTGACAGGAAGAGGTGAGA |
| acidic protein | | (SEQ ID NO: 28) | (SEQ ID NO: 29) |
| (GFAP) | | | |
| Human alpha- | MN136523.1 | TGGCTTTTCGGACTACACCC | TTCGAAGGCCGTGAACGTAA |
| herpesvirus 1 | | (SEQ ID NO: 30) | (SEQ ID NO: 31) |
| strain Macintyre | | | |

### Immunofluorescence

Cells grown in tissue culture plates or in 3D scaffolds were fixed in 4% paraformaldehyde and washed with 1×phosphate-buffered saline (PBS). Samples were incubated with a blocking buffer consisting of PBS, 10% goat serum, and 0.1% Triton X-100. Primary antibodies were added to the blocking buffer and incubated with samples overnight at 4° C. The next day, samples were washed several times with PBS and incubated with a corresponding fluorescently conjugated secondary antibody in a blocking buffer for 1 hour at room temperature. Nuclei were counterstained with 4′,6-diamidino-2-phenylindole (Invitrogen). All antibodies used in this study are listed in Table 2.

TABLE 2
Antibodies used for immunostaining.

| HOST | ANTIGEN | VENDOR | CATALOG # |
|---|---|---|---|
| Mouse | HSV Type1/2 gB | Thermofisher | MA1-19265 |
| Rabbit | HSV-1 | Abcam | ab9533 |
| Rabbit | Beta III tubulin (TUJ1) | Abcam | ab18207 |
| Mouse | Beta III tubulin (TUJ1) | Sigma | T8578 |
| Rabbit | Cleaved Caspase 3 (CC3) | RND | AF835 |
| Rabbit | Amyloid Fibril | Abcam | ab201062 |
| Mouse | Phospho-Tau (Ser202, | Thermofisher | MN1020 |
| | Thr205) (AT8) | | |
| Mouse | Microtubule-associated | Sigma | MAB3420 |
| | protein tau (Tau-1) | | |
| Mouse | Glial fibrillary acidic protein | Sigma | G3893 |
| | (GFAP) | | |
| Rabbit | Glial fibrillary acidic protein | Sigma | G9269 |
| | (GFAP) | | |
| Rabbit | Tumor necrosis factor alpha | Cell Signaling | 8184S |
| | (TNFα) | Technology | |
| Goat (Alexa 488 | Rabbit IgG | Thermofisher | A-11070 |
| conjugated) | | | |
| Goat (Alexa 584 | Rabbit IgG | Thermofisher | A-11072 |
| conjugated) | | | |
| Goat (Alexa 488 | Mouse IgG | Thermofisher | A-11017 |
| conjugated) | | | |
| Goat (Alexa 594 | Mouse IgG | Thermofisher | A-11020 |
| conjugated) | | | |

### Aβ ELISA Kit

A0340 and A0342 Human enzyme-linked immunosorbent assay (ELISA) kits were purchased from Thermo Fisher Scientific. Conditioned media (CM) harvested from mock- and HSV-1-infected hiNSCs were filtered and then subjected to ELISA assay according to the manufacturer's instructions.

### Microscopy

Fluorescent images were obtained using a Keyence BZ-X700 microscope and associated software. Bright-field and fluorescent images were obtained using a Keyence BZ-X700 microscope and associated software. Fluorescent images of 3D samples were taken using a Leica TCS FLIM SP8 (Wetzlar, Germany) confocal microscope. Scanning electron microscopy (SEM) was performed using a Zeiss Evo MA10 (Carl Zeiss Microscopy, Germany) to visualize the morphological properties of 3D brain-like tissue samples. Constructs were thoroughly dried and cross-sectioned and then gold-sputter-coated to prepare for subsequent SEM analysis.

### Electrophysiology

Function was assessed by sampling spontaneous electrical activity within mock- and HSV-1-infected samples. Local field potentials (LFPs) were collected using methods consistent with our previous electrophysiological experiments. Briefly, samples were placed into 35-mm plastic petri dishes containing 2 ml of extracellular solution: 130 mM NaCl, 1.25 mM NaH₂PO₄, 1.8 mM MgSO₄, 1.6 mM CaCl₂), 3 mM KCl, 10 mM Hepes-NaOH, and 5.5 mM glucose (pH 7.4). Dishes were placed onto a WP-16 Warmed Platform (with a TC-134A Handheld Temperature Controller), which maintained physiological temperature (37° C.). Next, samples were secured to the bottom of the dish with a fixed depressor to eliminate signal artifacts associated with the movement of the scaffold. A recording electrode was then positioned within the collagen hydrogel along silk fibers to measure LFPs. Electrodes were fabricated using borosilicate glass pipettes (length, 5 cm; inner diameter, 0.86 mm; outer diameter, 1.5 mm), which were pulled with a Sutter P-97 (Navato, CA, USA). Each glass pipette was pulled to the following specifications: <1 μm in tip diameter, 5- to 8-mm taper, and 40- to 80-megaohm resistance. Before each recording, impedance was measured where 100 megaohm was considered suitable for measurement. LFPs (mV) were collected (Axon Instruments DAC; Intan digital amplifier) with a sampling frequency of 2500 Hz for 2 min to assess the presence of spontaneous electrical activity. Data were collected using Clampex 10.7 (Axon Instruments) and exported to Clampfit 10.7, and traces were processed to extract spontaneous spikes (˜1 ms, ±0.35 mV). Spikes per minute were then computed and compared across groups.

### Statistics

All data are expressed as means±SD, and at least three biological replicates were analyzed per experiment. Independent experiments were repeated three times. Data with statistically significant differences were determined by one-factor analysis of variance (ANOVA) with post hoc Tukey test using the statistics software SYSTAT12 (Systat). P<0.05 was considered significant.

### Results:

hiNSCs are Highly Infectable by HSV-1

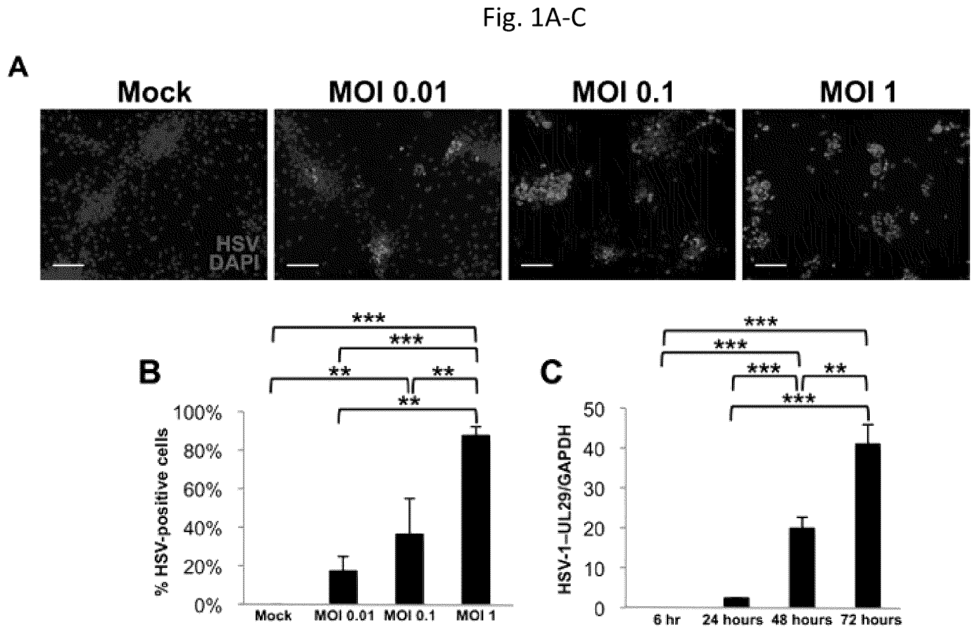

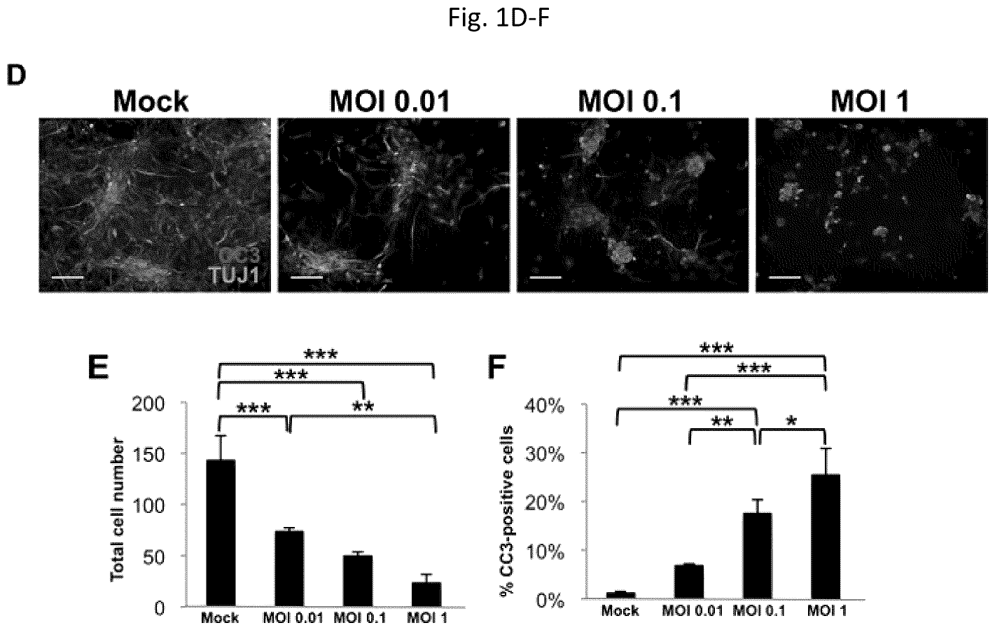

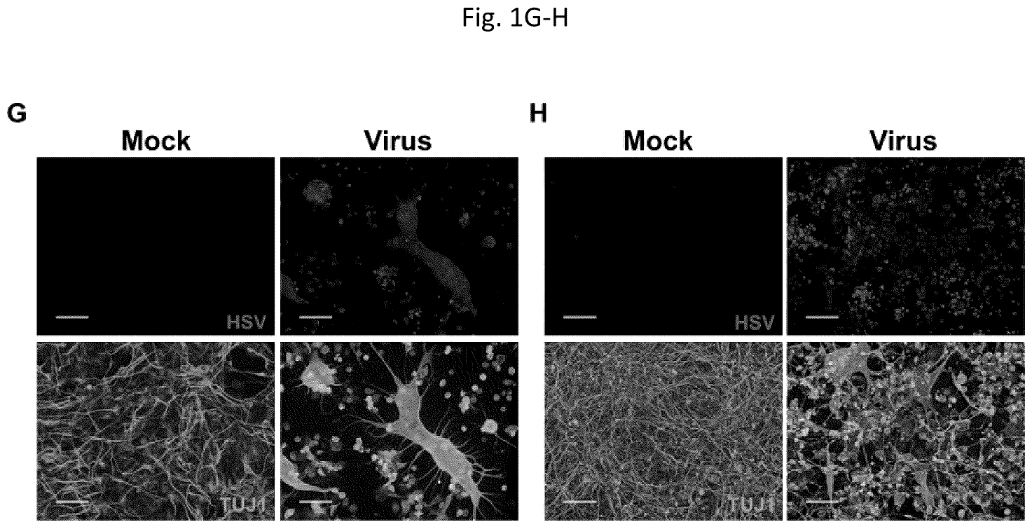

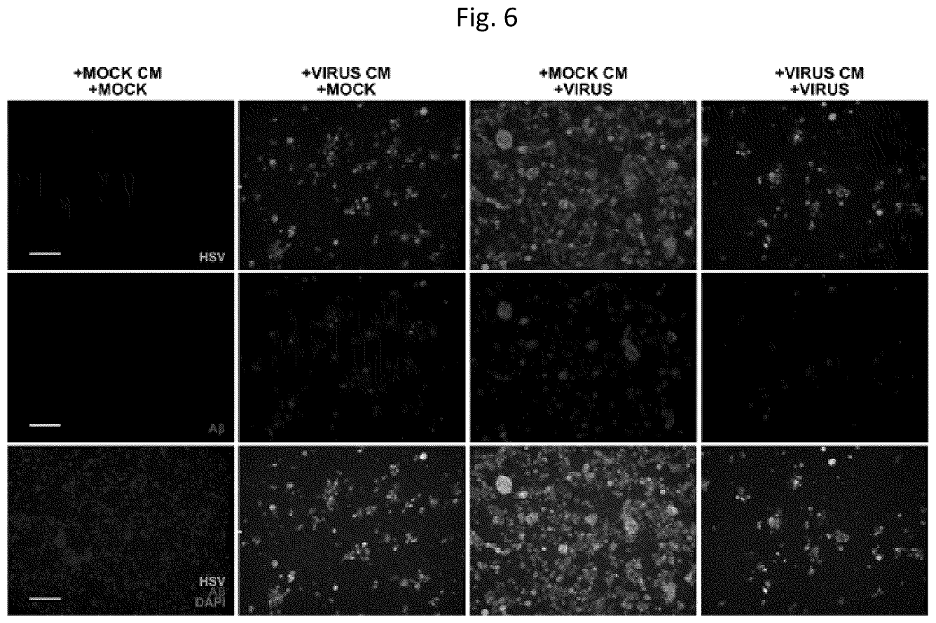

We first aimed to determine whether hiNSCs were infectable by HSV-1 and to establish an infection regimen for subsequent experiments. hiNSCs were exposed to mock or HSV-1 (MOI of 0.01 to 1) for 24 hours and then subsequently immunostained to identify HSV-1-infected cells (FIGS. 1, A and B). At an MOI of 1, nearly 100% of cells were infected after 24 hours. We also determined that expression of HSV-1-UL29 increased over time in culture (FIG. 1C). Furthermore, we demonstrate that virus-infected hiNSCs secrete HSV-1 that is capable of infecting new cells (FIG. 6). CM were harvested and filtered from mock- or HSV-1-infected hiNSCs that had been cultured for 2 days. Mock or fresh HSV-1 virus was added to CM, and new hiNSCs were cultured for 4 days. In virus CM samples without additional virus, there were HSV-1-positive cells, suggesting that infected hiNSCs secrete active HSV-1. Together, these data demonstrate how low-level viral inoculations lead to high levels of infection over time, which is reminiscent of chronically reactivating HSV-1 infections in patients.

High Level of HSV-1 Infection Results in Cell Death, while Low-Level Infection Induces Profound Morphological Changes in hiNSCs

Having established that hiNSCs are highly infectable even at very low MOIs, we also wanted to discern the effect of HSV-1 infection on apoptosis in hiNSCs. hiNSCs were exposed to mock or HSV-1 (MOI of 0.01 to 1) for 24 hours and then subjected to immunostaining against apoptosis marker, cleaved Caspase3 (CC3) (FIG. 1D). We found that increasing MOI resulted in fewer total cells (FIG. 1E) and increased apoptosis (FIG. 1F). Given that even a very low MOI of 0.01 caused substantial cell death in 24 hours, we performed subsequent HSV-1 infections at an MOI of 0.0001. We cultured hiNSCs for 1, 4, or 7 days before HSV-1 exposure for 2 to 3 days and subsequent immunostaining against HSV-1 and pan-neuronal marker β-III tubulin (TUJ1) (FIG. 1, G to I). We found that low-level HSV-1 infection caused profound morphological changes in hiNSCs. Briefly, infected hiNSCs formed large multicellular structures that retained neurite extensions, reminiscent of previously described syncytia present in the extracellular space of AD brains (36). This type of cell fusion has also been reported in a monkey kidney epithelial cell line in response to HSV-1 infection (37). The more immature the neurons at the time of infection, the larger the conglomerate structure formed. This result suggests that relatively high HSV-1 infection results in cell death, while low-level infection formed large, multicellular structures.

hiNSCs Produce Aβ+ PLFs and Specifically Regulate AD Mediators in Response to HSV-1 Infection

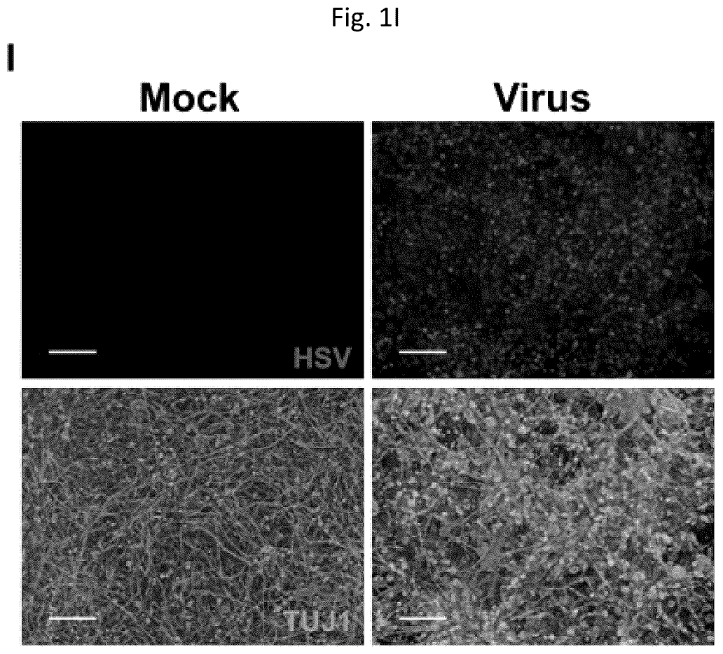

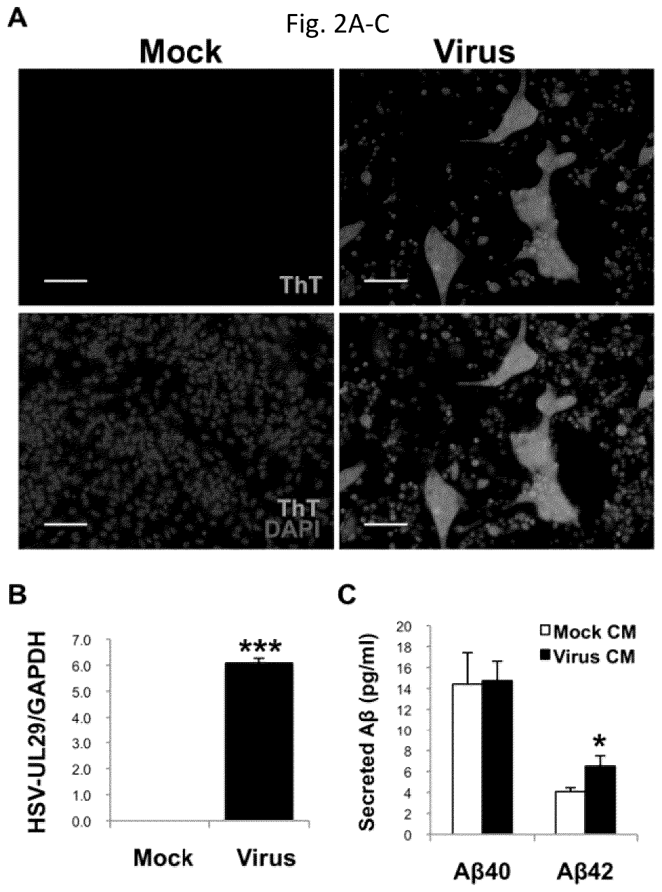

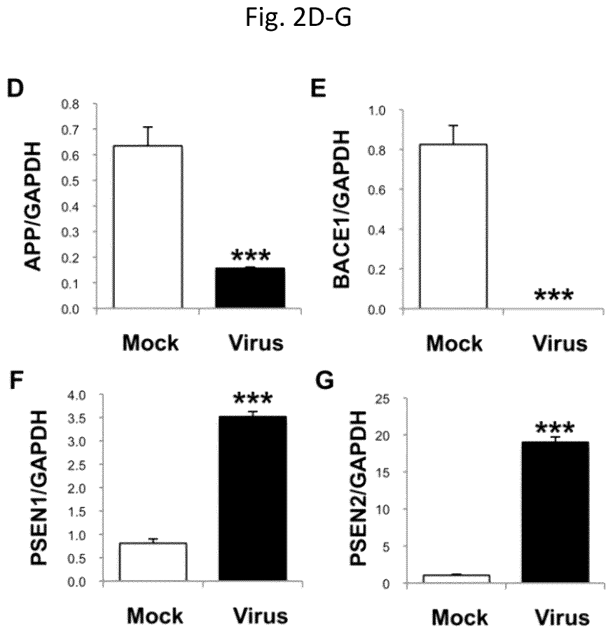

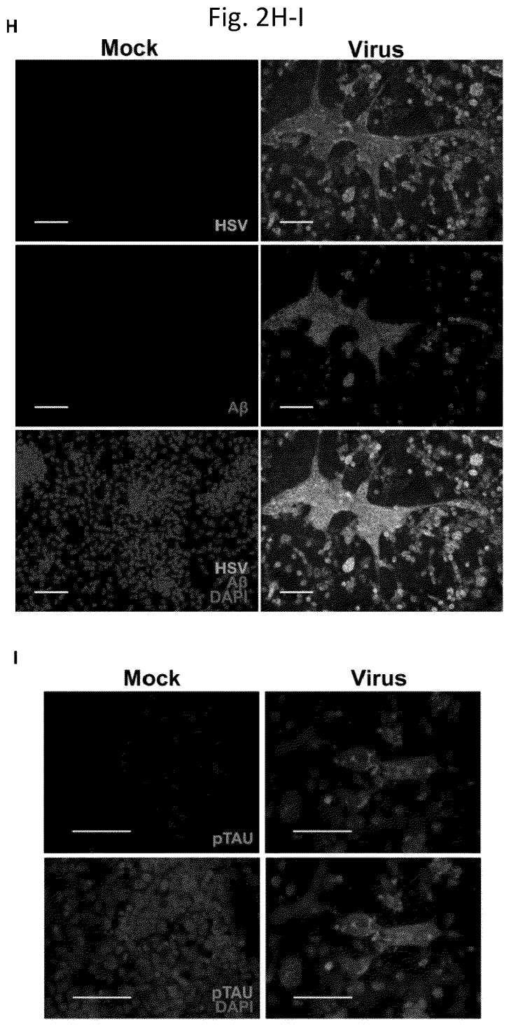

Next, we wanted to characterize the structures that formed in response to low-level HSV-1 infection in hiNSCs. Thioflavins are histological dyes used for visualizing protein aggregation. Thioflavin T (ThT) is a benzothiazole dye that exhibits enhanced fluorescence (emission, 445 to 482 nm) upon binding to amyloid fibrils and is considered the “gold standard” for identifying these structures in clinical samples (38). We cultured hiNSCs for 4 days and then subjected them to mock or low-level HSV-1 infection (MOI of 0.0001) for 3 days before subsequent fixation and ThT histological staining (FIG. 2A). These multicellular structures formed in HSV-1-infected hiNSCs stained strongly positive for ThT, suggesting the presence of amyloid fibrils within these structures. We confirmed HSV-1 infection by qPCR (FIG. 2B) and went on to further characterize the virally induced protein aggregates by performing an ELISA against the Aβ isoforms AP 1-40 and Aβ1-42 using CM from mock- or HSV-1-infected hiNSCs (FIG. 2C). We found a statistically significant increase in Aβ1-42, but not Aβ1-40, in HSV-1-infected CM, which is consistent with AD-like pathology.

We performed additional qPCR to ascertain whether known mediators involved in AD were also involved in the HSV-1-induced generation of PLFs in hiNSCs. qPCR analysis demonstrated that HSV-1 caused down-regulation of APP and BACE1 (FIGS. 2, D and E), as well as up-regulation of PSEN1/2 (FIGS. 2, F and G) in infected hiNSCs when compared to uninfected controls.

To further characterize the PLFs that formed in response to HSV-1 infection in hiNSCs, we immunostained against HSV and Aβ fibrils (FIG. 2H) to determine whether infected cells specifically expressed Aβ protein. After 3 days of low-level HSV-1 infection (MOI of 0.0001), nearly all hiNSCs were infected, with a portion of those infected cells expressing Aβ almost exclusively in regions of PLFs. The average number of nuclei within each individual PLF was 19, suggesting that these PLFs were composed of multiple cells.

As Aβ plaques are not the only pathological features of AD, we also assayed for the presence of NFTs, which are formed by the hyperphosphorylation and subsequent aggregation of a microtubule-associated protein known as tau. We immunostained mock- and virus-infected samples using a phospho-Tau antibody that specifically recognizes paired helical filament-Tau with phosphorylated residues Ser²⁰²/Thr²⁰⁵ (FIG. 2I). In HSV-1-treated hiNSCs, strongly positive Tau staining is visible on or near PLFs.

HSV-1 Infection Results in Increased Gliosis and Neuroinflammation in hiNSCs

We have previously demonstrated that hiNSCs spontaneously differentiate into neurons and glial fibrillary acidic protein (GFAP)-positive glia (28). Given that HSV-1 infection resulted in induction of PLFs similar to those found in patients with AD, we also aimed to discern whether other phenotypic characteristics of AD were induced in HSV-1-infected hiNSCs, such as reactive gliosis and up-regulation of pro-inflammatory cytokines. We cultured hiNSCs for 4 days, before subjecting to mock or HSV-1 (MOI of 0.0001) for 3 days, and then performed subsequent analyses.

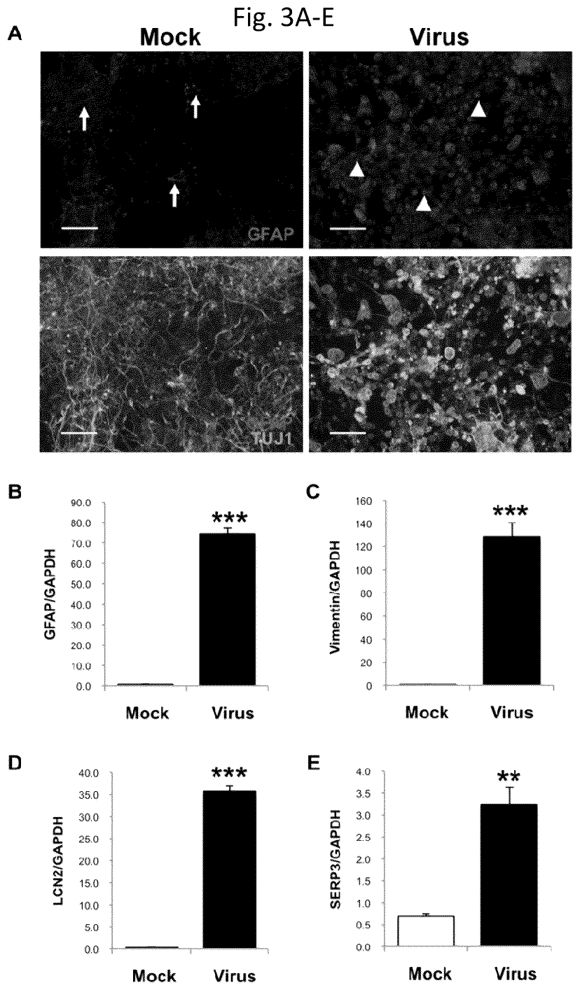

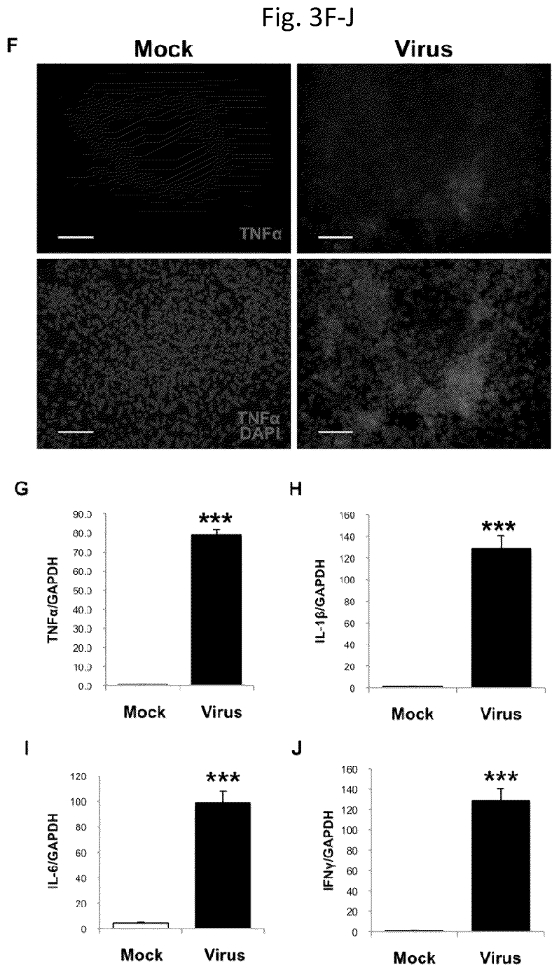

We first stained using an antibody against GFAP (FIG. 3A). In mock-infected hiNSCs, ˜9% of cells demonstrated GFAP-positive staining after 1 week in culture (arrows), consistent with previous reports (28). HSV-1-infected hiNSCs expressed ˜71% GFAP positivity (arrowheads), which was consistent with qPCR results (FIG. 3B). Furthermore, these GFAP-positive cells induced by HSV-1 have a swollen, globular morphology similar to that of gemistocytic astrocytes (39). The altered morphology is reminiscent of what is observed in postmortem tissue from AD patients with numerous clusters of GFAP-positive astrocytes that become more confluent as the disease progresses (7). We also tested other known markers of reactive astrocytes, vimentin, Lipocalin2 (LCN2), and Serpina3 (SERP3) (FIG. 3, C to E) and found that these markers were all highly up-regulated in response to HSV-1 infection in hiNSCs.

We also aimed to assay markers of neuroinflammation. Tumor necrosis factor-α (TNFα) is one of the major pro-inflammatory cytokines known to play a role in neurodegenerative diseases (40). We found that TNFα was up-regulated in HSV-1-infected hiNSCs as indicated by immunostaining and qPCR (FIGS. 3, F and G). Other known inflammatory mediators involved in AD are interleukin-1β (IL1β), IL-6, and interferon γ (IFNγ) (8). We assayed for expression of these markers and observed high levels of expression in virus-infected hiNSCs compared to uninfected controls.

VCV Treatment Results in Decreased HSV Infection, Decreased Aβ+ Plaque-Like Formation, and Normalized Expression of AD Mediators in Infected hiNSCs

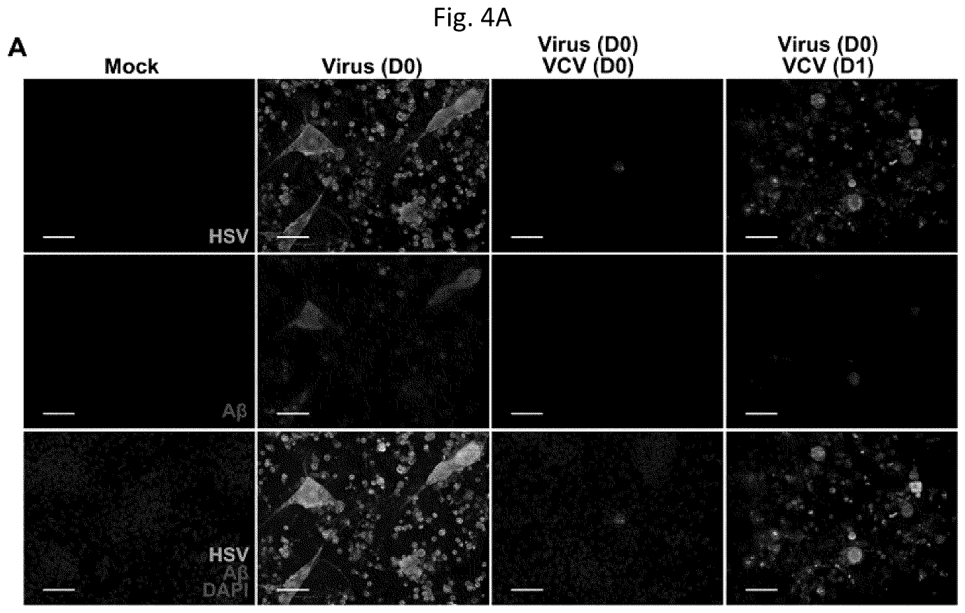

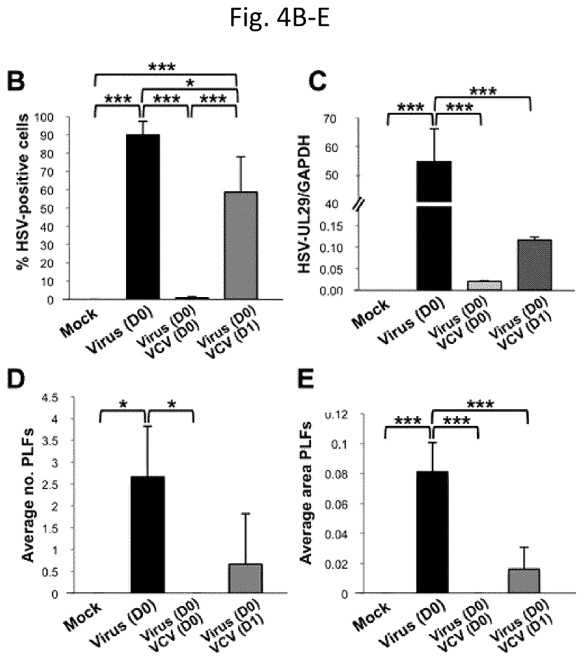

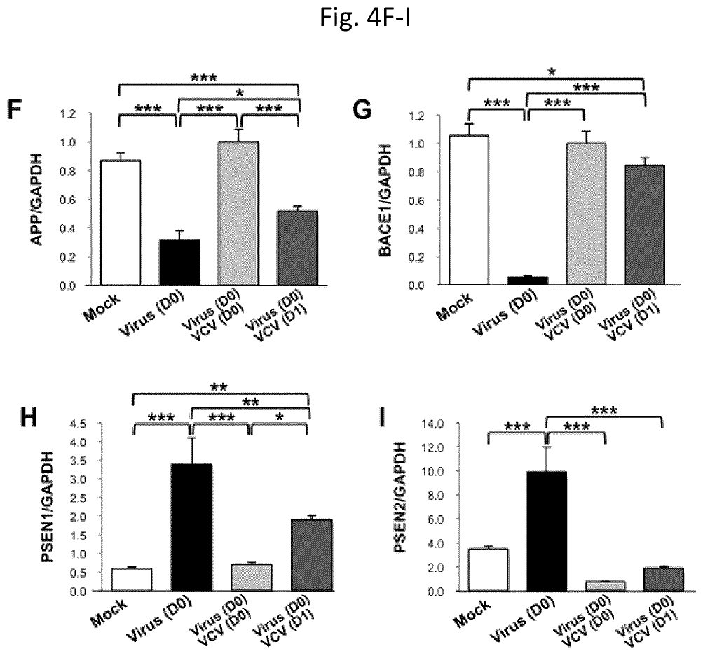

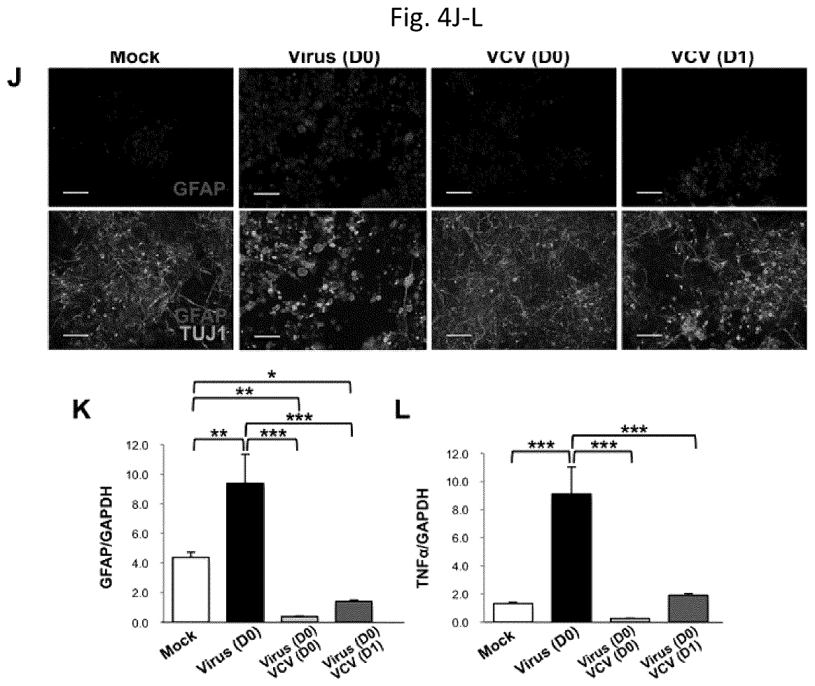

Given the data to suggest that HSV-1 directly caused an AD-like phenotype in hiNSCs, we wanted to determine whether inhibiting the virus using established antiviral medication would prevent or reduce the resulting AD phenotype. One of the most commonly used antiviral agents for treating HSV-1 infections is VCV, which specifically targets viral DNA replication in infected cells. We cultured hiNSCs for 4 days before subjecting to either mock or HSV-1 infection (MOI 0.0001). In addition, we added either vehicle or 200 μM VCV at D0 or D1 to observe potential changes with regard to timing of drug administration in the acquisition of an AD-like phenotype. hiNSCs treated with vehicle or VCV alone in the absence of virus had no discernible effect (data not shown). We first immunostained against HSV and Aβ (FIG. 4A) to determine any differences in the rate of HSV-1 infection and PLF generation in response to drug treatment. As expected, VCV caused a significant decrease in HSV-1 infection using both drug regimens as indicated by immunostaining (FIG. 4B) and qPCR (FIG. 4C); however, VCV treatment at D0 almost completely abolished infection. VCV treatment also significantly reduced the average number and size of PLFs in HSV-1-infected hiNSCs (FIGS. 4, D and E). We went on to assay the expression of previously characterized AD mediators-such as APP, BACE1, PSEN1, and PSEN2 (FIG. 4, F to I)—and found that VCV treatment at D0 nearly rescued all markers back to control values, while D1 VCV treatment showed partial rescue.

VCV Treatment of HSV-1-Infected hiNSCs Results in Reduced Gliosis and Neuroinflammation

We also aimed to determine whether VCV treatment had a similar effect in reducing HSV-1-induced gliosis and neuroinflammation in hiNSCs. We performed parallel experiments using the previously described dosing regimens and then immunostained for glial marker GFAP as well as TUJ1 to visualize neurons. As observed in previous experiments, HSV-1 induced robust glial formation (FIG. 4J) in hiNSCs compared to controls. VCV treatment at D0 almost completely restored GFAP expression to mock-treated samples as indicated by both immunostaining and qPCR (FIG. 4K). We also found that VCV treatment in HSV-1-infected hiNSCs significantly reduced the pro-inflammatory mediator TNFα at both D0 and D1 dosing regimens.

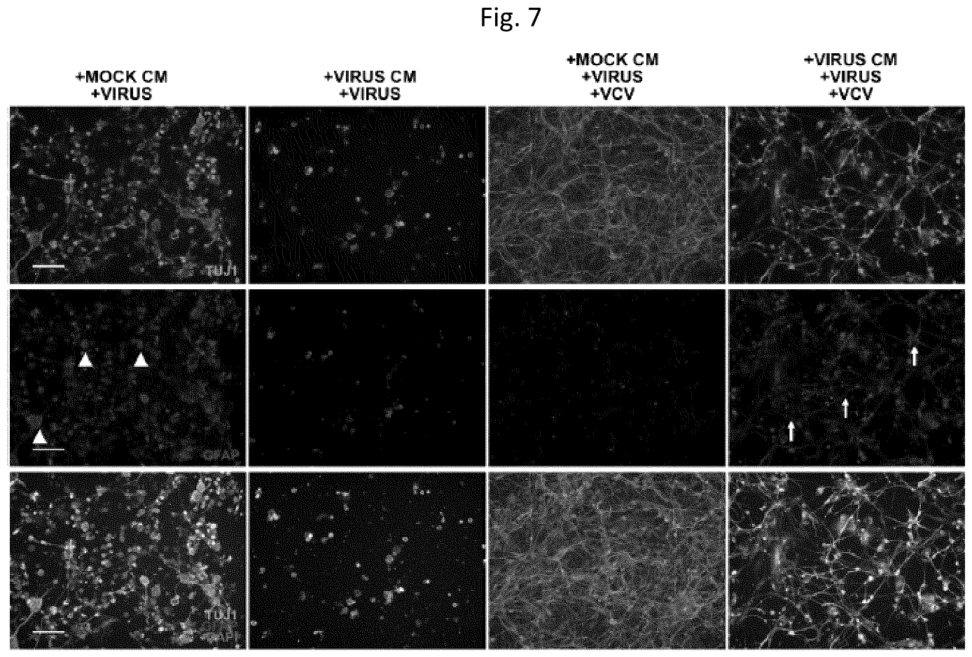

Moreover, we aimed to understand whether HSV-1-secreted factors could cause gliosis even in the absence of infection (FIG. 7). CM were harvested and filtered from mock- or HSV-1-infected hiNSCs that had been cultured for 2 days. Fresh HSV-1 virus was added to CM in the absence or presence of antiviral treatment VCV, and new hiNSCs were cultured for 4 days. β-III tubulin (TUJ1) and GFAP immunostaining was performed to visualize neurons and glia, respectively. Preventing HSV-1 infection using VCV in virus CM resulted in increased reactive glia formation, suggesting that HSV-1-induced factors play a role in astrogliosis even when no virus is active. This suggests that an initial low-level infection is sufficient to initiate a cascade of neurodegenerative events that is independent of the initial HSV-1 infection.

hiNSCs Cultured in 3D Brain Model Develop AD-Like Phenotype in Response to Low-Level HSV-1 Infection

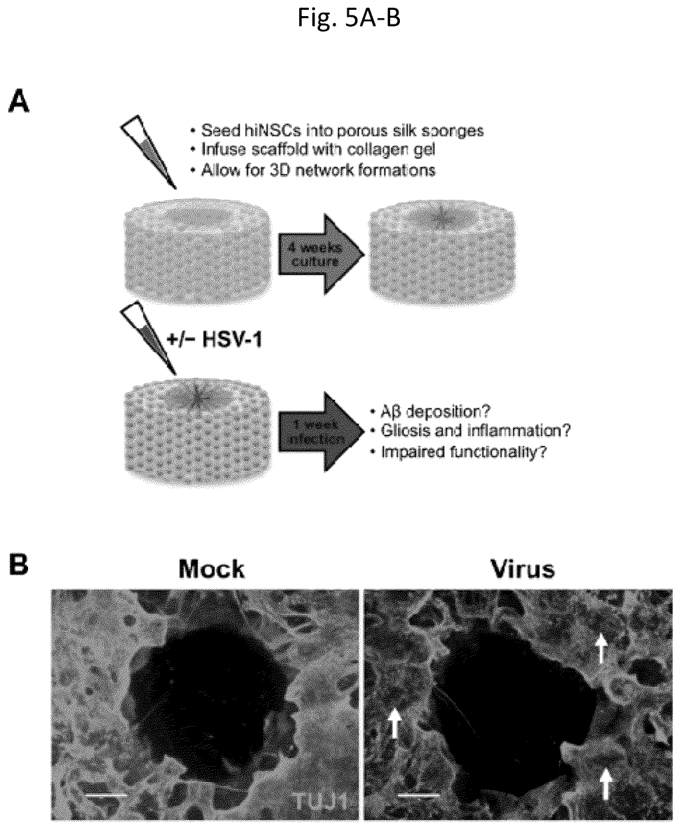

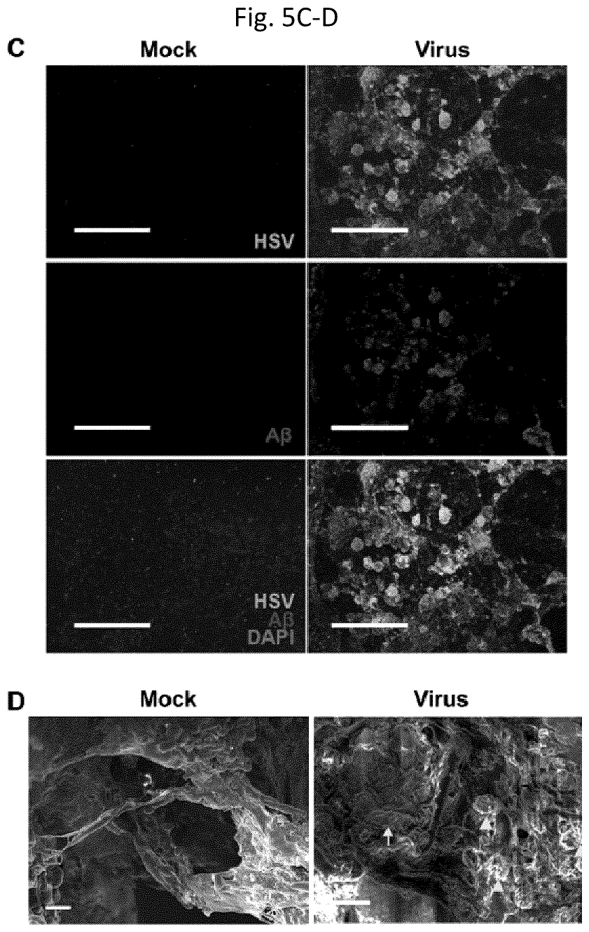

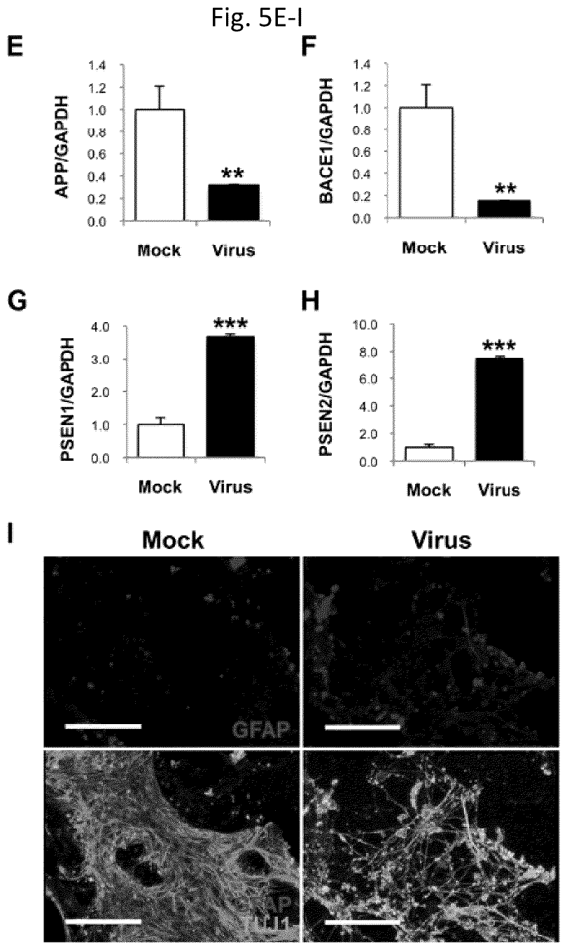

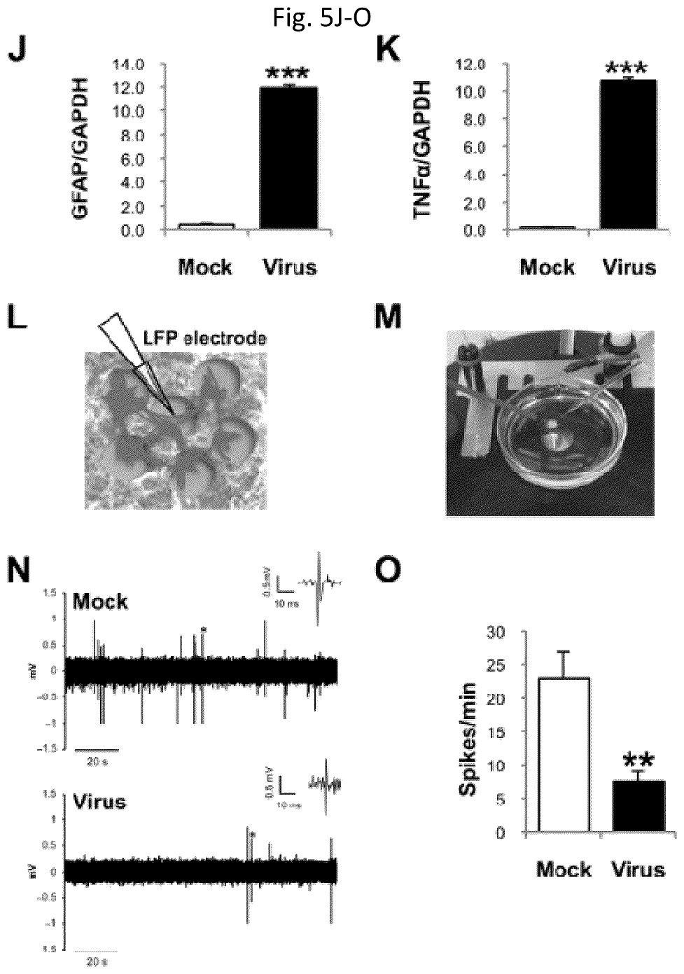

With the observation of robust AD-like phenotypes in our monolayer cultures, we pursued our goal of generating a 3D human brain model of herpes-induced AD. To achieve this goal, we used our established silk protein scaffold-based system, which was previously described (28, 30). We first developed a seeding and infection protocol (FIG. 5A). Briefly, silk porous laminin-coated scaffolds (height, 2 mm; outer diameter, 6 mm; inner diameter, 2 mm) were seeded with 10⁶ hiNSCs per sample, which were allowed to adhere overnight. The following day, the porous donuts were infused with type I collagen and allowed to stabilize over the course of 4 weeks in culture. These in vitro brain tissues were then subjected to mock or low-level HSV-1 infection (MOI of 0.0001) for 1 week before subsequent analyses.

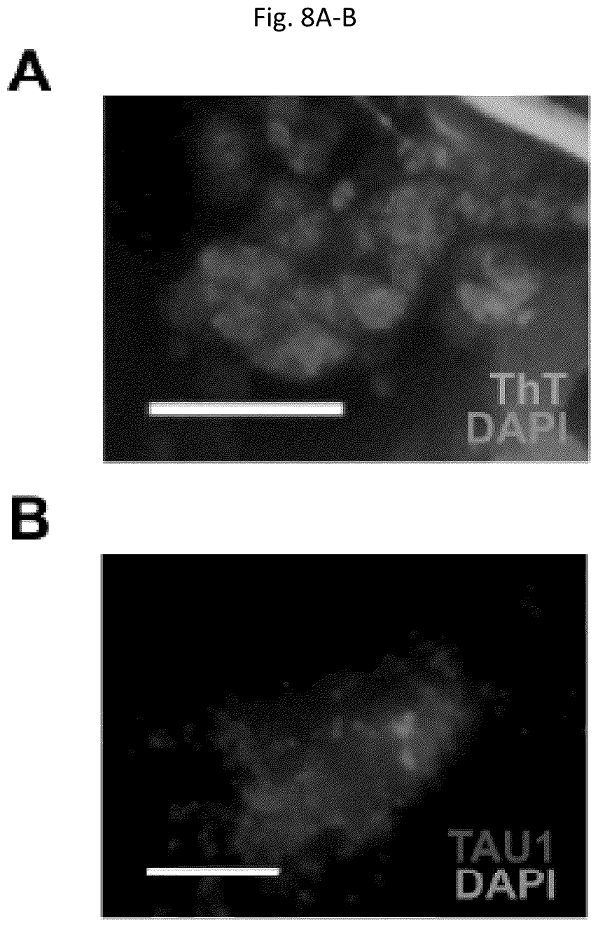

We fixed samples and immunostained against pan-neuronal marker TUJ1 to visualize growth of brain-like tissue in our model (FIG. 5B). After 1 week of HSV-1 infection, regions of neuronal loss were visible even at low magnification (arrows). We wanted to first confirm the penetration of the virus by immunostaining (FIG. 5C). We also wanted to understand whether observations made in monolayer cultures would also be observed in our 3D brain tissue model. Robust Aβ⁺ PLFs were observed only in the HSV-1-infected samples. We also performed SEM on the 3D human brain-like tissue constructs and found morphological changes in HSV-1-infected samples, namely, the presence of both small and large PLF structures (FIG. 5D), that were reminiscent of AD plaques. Upon closer examination of PLFs by confocal microscopy, these structures also stained positive for ThT and Tau1 (FIG. 8).

We went on to assay the expression of previously characterized AD mediators—such as APP, BACE1, PSEN1, and PSEN2 (FIG. 5, F to H)—and found that these mediators were similarly regulated as in monolayer cultures, with significant decreases in both APP and BACE1, and a corresponding increase in PSEN1/2. We further wanted to discern whether reactive gliosis and inflammation were occurring in these 3D cultures as in our previous experiments. In accordance with previous observations, GFAP expression was significantly increased as indicated by both immunostaining (FIG. 51) and qPCR (FIG. 5J). Similarly, TNFα expression was also significantly up-regulated in HSV-1-infected hiNSC donuts (FIG. 5K) when compared to uninfected controls. To further understand this AD-like pathology, we performed a qPCR array of known AD mediators (Qiagen RT Profiler Array for AD) to compare expression levels between mock- and HSV-1-infected 3D human brain-like tissue constructs (FIG. 9 and Table 3). Forty genes in this AD-specific array were significantly up-regulated in HSV-1-infected samples.

TABLE 3
Genes upregulated in response to HSV-1 infection in 3D human brain-like tissue
constructs using the Qiagen RT Profiler Array for Alzheimer's Disease.

| | | | FOLD |
|---|---|---|---|
| SYMBOL | GENE NAME | ACCESSION # | UPREGULATION |
| CTSG | Cathepsin G | NM_001911 | 86.18 |
| MPO | Myeloperoxidase | NM_000250 | 42.32 |
| PRKCQ | Protein kinase C, theta | NM_006257 | 33.80 |
| IL1A | Interleukin 1, alpha | NM_000575 | 31.45 |
| CASP4 | Caspase 4, apoptosis-related cysteine peptidase | NM_001225 | 28.94 |
| SERP3 | Serpin peptidase inhibitor, clade A (alpha-1 | NM_001085 | 28.77 |
| | antiproteinase, antitrypsin), member 3 | | |
| CHAT | Choline O-acetyltransferase | NM_020985 | 28.29 |
| PLAU | Plasminogen activator, urokinase | NM_002658 | 27.38 |
| GNGT2 | Guanine nucleotide binding protein (G protein), gamma | NM_031498 | 22.98 |
| | transducing activity polypeptide 2 | | |
| INS | Insulin | NM_000207 | 19.45 |
| BACE2 | Beta-site APP-cleaving enzyme 2 | NM_012105 | 19.03 |
| GNGT1 | Guanine nucleotide binding protein (G protein), gamma | NM_021955 | 17.63 |
| | transducing activity polypeptide 1 | | |
| PLG | Plasminogen | NM_000301 | 15.68 |
| APOA1 | Apolipoprotein A-I | NM_000039 | 15.49 |
| ERN1 | Endoplasmic reticulum to nucleus signaling 1 | NM_001433 | 12.13 |
| APBA3 | Amyloid beta (A4) precursor protein-binding, family A, | NM_004886 | 10.91 |
| | member 3 | | |
| CDKL1 | Cyclin-dependent kinase-like 1 (CDC2-related kinase) | NM_004196 | 10.74 |
| PRKCI | Protein kinase C, iota | NM_002740 | 9.89 |
| GNG11 | Guanine nucleotide binding protein (G protein), gamma 11 | NM_004126 | 8.61 |
| NTRK1 | Neurotrophic tyrosine kinase, receptor, type 1 | NM_002529 | 7.33 |
| BDNF | Brain-derived neurotrophic factor | NM_001709 | 6.44 |
| INSR | Insulin receptor | NM_000208 | 6.05 |
| GSK3B | Glycogen synthase kinase 3 beta | NM_002093 | 5.55 |
| SNCB | Synuclein, beta | NM_003085 | 5.37 |
| ACHE | Acetylcholinesterase | NM_000665 | 4.25 |
| LRP6 | Low density lipoprotein receptor-related protein 6 | NM_002336 | 3.44 |
| UBQLN1 | Ubiquilin 1 | NM_013438 | 3.26 |
| A2M | Alpha-2-macroglobulin | NM_000014 | 2.96 |
| HPRT1 | Hypoxanthine phosphoribosyltransferase 1 | NM_000194 | 2.90 |
| GNB5 | Guanine nucleotide binding protein (G protein), beta 5 | NM_016194 | 2.78 |
| PRKCE | Protein kinase C, epsilon | NM_005400 | 2.71 |
| PKP4 | Plakophilin 4 | NM_003628 | 2.52 |
| PRKCD | Protein kinase C, delta | NM_006254 | 2.47 |
| PRKCZ | Protein kinase C, zeta | NM_002744 | 2.42 |
| PSEN2 | Presenilin 2 (Alzheimer disease 4) | NM_000447 | 2.37 |
| PRKCG | Protein kinase C, gamma | NM_002739 | 2.23 |
| IDE | Insulin-degrading enzyme | NM_004969 | 2.21 |
| UQCRC2 | Ubiquinol-cytochrome c reductase core protein II | NM_003366 | 2.17 |
| PRKCA | Protein kinase C, alpha | NM_002737 | 2.15 |
| BCHE | Butyrylcholinesterase | NM_000055 | 2.07 |

HSV-1-treated 3D human brain-like tissue cultures show significantly less electrophysiological activity reminiscent of impaired functionality in patients with AD

Last, the gold standard of any bioengineered tissue model is functionality. To be considered “brain-like tissue,” the tissue construct must generate and maintain electrical activity. Furthermore, we hypothesized that our herpes-induced AD brain model would also recapitulate patient symptoms with regard to diminished brain activity. High levels of Aβ oligomers have been shown to cause synapse malfunction (41) and reduce long-term potentiation, which correlates with memory loss (42).

LFP is an electrophysiological recording generated by the total electric current flowing by resident neurons present in a small area of nervous tissue. Voltage is produced across this region by synaptic activity, which can be measured and quantified. Briefly, a microelectrode is placed on the tissue, which reads the voltage change in that region over time. These relative voltage changes or “spikes” are indicative of actively firing neurons present in the tissue (FIGS. 5, L and M). LFP recordings were taken from both mock- and HSV-1-infected donut cultures maintained for 5 weeks. Representative traces (FIG. 5N) and the averages of spikes per minute for n=6 donuts per treatment (FIG. 50) demonstrated that HSV-1-treated donuts showed significantly less activity compared to controls, which is reminiscent of the impaired brain functionality seen in patients with AD.

### Discussion:

Generating in vitro human models of sporadic AD poses a considerable challenge because of the complex, undefined etiology and slow disease progression that naturally occurs in patients. Here, we describe a multidisciplinary approach to address this critical need by generating robust and physiologically relevant 3D human tissues for studying sporadic AD through the use of hiNSC technology and HSV-1 in combination with a bioengineered brain tissue model.

A number of in vitro human models of AD have been developed. Human-induced pluripotent stem cell (hiPSC) technology has provided a useful platform with which to study these types of neurodegenerative diseases; however, it is not without limitations. One benefit to the hiPSC methodology is the ability to convert specific patient cells to a pluripotent state. In this way, cells can be harvested from patients with known disorders and/or genetic mutations, reprogrammed, and then differentiated to essentially create a “disease in a dish.” A number of protocols have been developed to differentiate iPSCs into various neuronal phenotypes; however, these protocols are often very time consuming, require multiple complicated intermediate steps such as the formation of nonadherent embryoid bodies and neurospheres, and result in large variability in achieving neuronal differentiation (43). A number of studies have been conducted using cells derived from patients with familial EOAD, which is associated with mutations in APP, PSEN1, or PSEN2 (2). As these genetic mutations provide the stimulus for initiating AD in vitro, these studies do not address the issue of disease onset or progression in most AD cases, which occur sporadically. Furthermore, because the process by which somatic cells are converted into iPSCs is thought to epigenetically “reset” the cells into an embryonic-like state, it has been suggested that these cell lines lack an aging profile upon reprogramming. Studies using iPSC-derived neurons demonstrate an immature phenotype and a lack of aging markers (44), which poses an issue when attempting to model a neurodegenerative disease in which the primary risk factor is age. It has been proposed that direct reprogramming or transdifferentiation, methods that bypass the pluripotent state, more efficiently maintains an aging signature in resulting neurons (45).

Other studies have taken alternative approaches to generate these types of human disease models by synthetically creating an AD-like microenvironment. While some studies have shown certain characteristics of AD, such as neuronal loss (46) or Tau phosphorylation (47), most of these models are often unable to fully recapitulate the more complex features of AD, such as plaque formation, reactive gliosis, and loss of neural tissue functionality. In addition, to generate some of these neurodegenerative models, many of these systems require months of culture before establishing any discernible phenotype (33). Perhaps, most, if not all, studies to date specifically require the incorporation of known AD mediators to initiate an AD-like state. For example, multiple studies induce AD in their respective model systems through the use of viruses overexpressing AD mediators such as PSEN1 (33), pro-inflammatory cytokines (48), and/or incorporation of detrimental Aβ peptides (33, 35, 48). The requirement of the use of exogenous mediators of AD to artificially induce plaque formation is not reflective of the in vivo condition, especially considering that the actual cause of AD is so elusive.

There is increasing evidence to suggest a clinical correlation between pathogens such as herpes and the development of sporadic AD (20, 21), including the findings that HSV-1 DNA was detected in the brain tissue of patients with AD (24-26) and recent epidemiological studies even reporting HSV-1 infection as a significant risk factor of later AD onset (27). While there is no published correlative evidence of active herpes infections in patients with AD, these clinical findings suggest that active infection may not be a requirement for AD, but rather that prior history of herpes infection is sufficient to initiate AD pathogenesis. Ours is not the first to describe an experimental link between HSV-1 and AD. Several reports have described in vitro monolayer cell culture models in which HSV-1 has led to neuronal death (32, 49), Tau phosphorylation (50), and intracellular expression of various isoforms of APP cleavage products (51-53). While the finding that certain proteins known to be involved in the amyloidogenic pathway are also up-regulated in response to HSV-1 infection is exciting, few studies have reported the formation of multicellular plaque-like structures in neuron cultures. These PLFs are composed of multiple merged infected hiNSCs, averaging ˜19 nuclei per individual PLF (data not shown). A recent study suggested an intracellular basis for the ultimate formation of these extracellular Aβ plaques (54). Briefly, small Aβ oligomers form and get transported to vesicular bodies, after which a phagocytic event occurs to generate multicellular plaques that apoptose releasing contents into the extracellular space. It is plausible that similar mechanisms occur in our HSV-mediated in vitro system, as the PLFs formed in these cultures are multicellular and apoptotic.

Furthermore, most of these HSV-AD studies have relied on the use of primary cells derived from rodent models (49, 52) and/or human cancer cell lines (50-53) and often require the use of exogenous AD mediators (51, 53). Recent work by Eimer et al. (34) reported that Aβ proteins sequestered herpes viruses in 3D human neural cell cultures using an immortalized cell line. However, this study required the use of lentiviruses overexpressing human b-APP or APP and PSEN1, containing familial AD mutations, which is not truly physiologically reflective of sporadic AD pathogenesis. In addition, this model did not demonstrate multiple aspects of AD physiology including multicellular plaque formation, reactive gliosis, or loss of brain tissue functionality.

While our study demonstrates multiple phenotypic characteristics of patients with AD, it was somewhat unexpected that BACE1 expression was decreased in response to HSV-1 infection, a finding that was consistent across all experiments. We demonstrated a significant decrease in APP expression, which aligns with results from previous research (51). Two enzymes, β-secretase and γ-secretase, sequentially cleave transmembrane protein APP to generate Aβ peptides. As the initial BACE1-mediated cleavage event is believed to initiate Aβ production, inhibition of this enzyme has emerged as a target for therapeutic treatment of AD. Because HSV-1 causes the production of Aβ1-42 in hiNSCs, given its role in the amyloidogenic processing of the APP protein, the hypothesis is that HSV-1 would also increase BACE1 expression (2) as it has previously in HSV-1-infected Vero cell cultures (31). BACE1 functions in other capacities outside of APP regulation. BACE1 KO (BACE1^{-/-}) mice have been reported to exhibit smaller size, decreased survival, spontaneous seizures (55), and memory deficits (56). BACE1 is highly expressed in the developing nervous system, suggestive of its critical role in neuronal proliferation, differentiation, and maturation. In addition, conditional KO of BACE1 in adult animals resulted in disorganization of the hippocampus, suggesting that BACE1 inhibitor drugs developed for AD treatment might have off-target detrimental effects on learning and memory (57), such as neuronal differentiation. Because BACE1 is so intricately involved in many of these developmental processes, we postulate that, in our neural stem cell system, BACE1 functions in other capacities outside of APP regulation and that the HSV-1-induced decrease in BACE1 expression is the consequence of impaired differentiation of hiNSCs.

To identify potential factors that may compensate for BACE1 in APP regulation, we performed a qPCR array of known AD mediators (Qiagen RT Profiler Array for AD) to compare expression levels between mock- and HSV-1-infected 3D brain-like tissue constructs (FIG. 9 and Table 3). Forty genes in this AD-specific array were significantly up-regulated in HSV-1-infected 3D brain-like constructs. Of particular interest were cathepsin G (CTSG) (86.18-fold up-regulation) and BACE2 (19.03-fold up-regulation) (Table 3). These two AD-associated factors, which are both highly up-regulated by HSV-1 in 3D human brain-like constructs, have both been suggested to play similar roles to BACE1 in AD pathogenesis. CTSG was shown to cleave APP, and its expression has been localized to the temporal cortex of both AD and aged human brain tissue, often associated with Aβ plaques (58). In addition, while the role of BACE2, a β-secretase that has 45% homology with BACE1, has often been debated, several recent reports have directly implicated BACE2 with AD pathogenesis. Unexpectedly high levels of BACE2 expression were identified in AD brain tissue, demonstrating higher enzymatic activity in samples taken from patients at preclinical stages of AD with expression colocalizing with reactive astrocytes (59). Furthermore, BACE2 was also recently shown to function as a conditional β-secretase as a novel mechanism of AD pathogenesis (60). Together, we suggest that BACE1 is down-regulated in our system, likely because of its prominent role in differentiation of neural stem cells, and that CTSG and/or BACE2 are likely candidates that compensate for this lack of BACE1 expression by functioning in the primary cleavage event of APP that initiates Aβ plaque generation and AD pathogenesis.

While previous reports have suggested a potential link between HSV-1 and up-regulation of certain AD mediators (31), to date, no one has shown direct causality of AD by herpes virus in a 3D human brain model. Furthermore, ours is the first to report a human model of AD that displays so many physiologically relevant features of human disease in one system, including the generation of large, multicellular, dense Aβ⁺ fibrillar PLFs, neuronal loss, reactive gliosis, neuroinflammation, and diminished neural network functionality. We also demonstrate efficacy in reducing herpes-induced AD phenotypes through the use of clinically relevant antiviral medication, suggesting that this type of pharmaceutical intervention might be useful for preventing and/or treating AD in patients. Importantly, our 3D human brain-like tissue model requires a shorter time period to achieve an AD-like phenotype relative to other 3D systems of AD (33) and uses unmodified stem cells completely in the absence of any established mediators known to induce AD specifically (33-35). Together, we describe a rapid and reproducible 3D human brain-like tissue model of herpes-induced sporadic AD, which yields a physiologically relevant phenotype that closely mimics pathological findings in human patients. This robust bioengineered system will provide the opportunity to further elucidate mechanisms of sporadic AD pathogenesis and to ultimately develop more efficacious strategies for treating this complex and devastating disease.

### REFERENCES

1. Alzheimer's Association, 2016 Alzheimer's disease facts and figures. Alzheimers Dement. 12, 459-509 (2016).
2. A. C. Naj, G. D. Schellenberg; Alzheimer's Disease Genetics Consortium (ADGC), Genomic variants, genes, and pathways of Alzheimer's disease: An overview. Am. J. Med. Genet. B Neuropsychiatr. Genet. 174, 5-26 (2017).
3. S. W. Weyer, M. Klevanski, A. Delekate, V. Voikar, D. Aydin, M. Hick, M. Filippov, N. Drost, K. L. Schaller, M. Saar, M. A. Vogt, P. Gass, A. Samanta, A. Jaschke, M. Korte, D. P. Wolfer, J. H. Caldwell, U. C. Muller, APP and APLP2 are essential at PNS and CNS synapses for transmission, spatial learning and LTP. EMBO J. 30, 2266-2280 (2011).
4. R. J. Kelleher III, J. Shen, Presenilin-1 mutations and Alzheimer's disease. Proc. Natl. Acad. Sci. U.S.A. 114, 629-631 (2017).
5. B. De Strooper, R. Vassar, T. Golde, The secretases: Enzymes with therapeutic potential in Alzheimer disease. Nat. Rev. Neurol. 6, 99-107 (2010).
6. R. H. Takahashi, T. Nagao, G. K. Gouras, Plaque formation and the intraneuronal accumulation of D-amyloid in Alzheimer's disease. Pathol. Int. 67, 185-193 (2017).
7. B. G. Perez-Nievas, A. Serrano-Pozo, Deciphering the astrocyte reaction in Alzheimer's disease. Front. Aging Neurosci. 10, 114 (2018).
8. G. J. Ho, R. Drego, E. Hakimian, E. Masliah, Mechanisms of cell signaling and inflammation in Alzheimer's disease. Curr. Drug Targets Inflamm. Allergy 4, 247-256 (2005).
9. P. H. Reddy, G. Mani, B. S. Park, J. Jacques, G. Murdoch, W. Whetsell Jr., J. Kaye, M. Manczak, Differential loss of synaptic proteins in Alzheimer's disease: Implications for synaptic dysfunction. J. Alzheimers Dis. 7, 103-117 (2005).
10. C. Arber, C. Lovejoy, S. Wray, Stem cell models of Alzheimer's disease: Progress and challenges. Alzheimers Res. Ther. 9, 42 (2017).
11. B. J. Balin, C. S. Little, C. J. Hammond, D. M. Appelt, J. A. Whittum-Hudson, H. C. Gerard, A. P. Hudson, Chlamydophila pneumoniae and the etiology of late-onset Alzheimer's disease. J. Alzheimers Dis. 13, 371-380 (2008).
12. J. Miklossy, Emerging roles of pathogens in Alzheimer disease. Expert Rev. Mol. Med. 13, e30 (2011).
13. R. F. Itzhaki, R. Lathe, B. J. Balin, M. J. Ball, E. L. Bearer, H. Braak, M. J. Bullido, C. Carter, M. Clerici, S. L. Cosby, K. del Tredici, H. Field, T. Fulop, C. Grassi, W. S. T. Griffin, J. Haas, A. P. Hudson, A. R. Kamer, D. B. Kell, F. Licastro, L. Letenneur, H. Lovheim, R. Mancuso, J. Miklossy, C. Otth, A. T. Palamara, G. Perry, C. Preston, E. Pretorius, T. Strandberg, N. Tabet, S. D. Taylor-Robinson, J. A. Whittum-Hudson, Microbes and Alzheimer's disease. J. Alzheimers Dis. 51, 979-984 (2016).
14. S. A. Harris, E. A. Harris, Herpes simplex virus type 1 and other pathogens are key causative factors in sporadic alzheimer's disease. J. Alzheimers Dis. 48, 319-353 (2015).
15. B. Readhead, J. V. Haure-Mirande, C. C. Funk, M. A. Richards, P. Shannon, V. Haroutunian, M. Sano, W. S. Liang, N. D. Beckmann, N. D. Price, E. M. Reiman, E. E. Schadt, M. E. Ehrlich, S. Gandy, J. T. Dudley, Multiscale analysis of independent alzheimer's cohorts finds disruption of molecular, genetic, and clinical networks by human herpesvirus. Neuron 99, 64-82.e7 (2018).
16. M. L. Giuffrida, F. Caraci, B. Pignataro, S. Cataldo, P. de Bona, V. Bruno, G. Molinaro, G. Pappalardo, A. Messina, A. Palmigiano, D. Garozzo, F. Nicoletti, E. Rizzarelli, A. Copani, β-amyloid monomers are neuroprotective. J. Neurosci. 29, 10582-10587 (2009).
17. J. Miklossy, Alzheimer's disease—a neurospirochetosis. Analysis of the evidence following Koch's and Hill's criteria. J. Neuroinflammation 8, 90 (2011).
18. D. Pisa, R. Alonso, A. Juarranz, A. Rabano, L. Carrasco, Direct visualization of fungal infection in brains from patients with Alzheimer's disease. J. Alzheimers Dis. 43, 613-624 (2015).
19. K. A. Bates, J. Fonte, T. A. Robertson, R. N. Martins, A. R. Harvey, Chronic gliosis triggers Alzheimer's disease-like processing of amyloid precursor protein. Neuroscience 113, 785-796 (2002).
20. R. F. Itzhaki, Corroboration of a major role for herpes simplex virus type 1 in Alzheimer's disease. Front. Aging Neurosci. 10, 324 (2018).
21. R. F. Itzhaki, Herpes simplex virus type 1 and Alzheimer's disease: Possible mechanisms and signposts. FASEB J. 31, 3216-3226 (2017).
22. H. Liu, K. Qiu, Q. He, Q. Lei, W. Lu, Mechanisms of blood-brain barrier disruption in herpes simplex encephalitis. J. Neuroimmune Pharmacol. 14, 157-172 (2019).
23. M. J. Ball, “Limbic predilection in Alzheimer dementia: Is reactivated herpesvirus involved?”. Can. J. Neurol. Sci. 9, 303-306 (1982).
24. G. A. Jamieson, N. J. Maitland, G. K. Wilcock, J. Craske, R. F. Itzhaki, Latent herpes simplex virus type 1 in normal and Alzheimer's disease brains. J. Med. Virol. 33, 224-227 (1991).
25. G. A. Jamieson, N. J. Maitland, G. K. Wilcock, C. M. Yates, R. F. Itzhaki, Herpes simplex virus type 1 DNA is present in specific regions of brain from aged people with and without senile dementia of the Alzheimer type. J. Pathol. 167, 365-368 (1992).
26. M. A. Wozniak, A. P. Mee, R. F. Itzhaki, Herpes simplex virus type 1 DNA is located within Alzheimer's disease amyloid plaques. J. Pathol. 217, 131-138 (2009).
27. R. F. Itzhaki, R. Lathe, Herpes viruses and senile dementia: First population evidence for a causal link. J. Alzheimers Dis. 64, 363-366 (2018).
28. D. M. Cairns, K. Chwalek, Y. E. Moore, M. R. Kelley, R. D. Abbott, S. Moss, D. L. Kaplan, Expandable and rapidly differentiating human induced neural stem cell lines for multiple tissue engineering applications. Stem Cell Reports 7, 557-570 (2016).
29. D. M. Cairns, D. S. S. K. Boorgu, M. Levin, D. L. Kaplan, Niclosamide rescues microcephaly in a humanized in vivo model of Zika infection using human induced neural stem cells. Biol. Open 7, bio031807 (2018).
30. K. Chwalek, M. D. Tang-Schomer, F. G. Omenetto, D. L. Kaplan, In vitro bioengineered model of cortical brain tissue. Nat. Protoc. 10, 1362-1373 (2015).
31. M. A. Wozniak, A. L. Frost, C. M. Preston, R. F. Itzhaki, Antivirals reduce the formation of key Alzheimer's disease molecules in cell cultures acutely infected with herpes simplex virus type 1. PLOS One 6, e25152 (2011).
32. K. Bourgade, A. Le Page, C. Bocti, J. M. Witkowski, G. Dupuis, E. H. Frost, T. Fulop Jr., Protective effect of Amyloid-β peptides against herpes simplex virus-1 infection in a neuronal cell culture model. J. Alzheimers Dis. 50, 1227-1241 (2016).
33. S. H. Choi, Y. H. Kim, M. Hebisch, C. Sliwinski, S. Lee, C. D'Avanzo, H. Chen, B. Hooli, C. Asselin, J. Muffat, J. B. Klee, C. Zhang, B. J. Wainger, M. Peitz, D. M. Kovacs, C. J. Woolf, S. L. Wagner, R. E. Tanzi, D. Y. Kim, A three-dimensional human neural cell culture model of Alzheimer's disease. Nature 515, 274-278 (2014).
34. W. A. Eimer, D. K. Vijaya Kumar, N. K. Navalpur Shanmugam, A. S. Rodriguez, T. Mitchell, K. J. Washicosky, B. Gyorgy, X. O. Breakefield, R. E. Tanzi, R. D. Moir, Alzheimer's disease-associated β-amyloid is rapidly seeded by herpesviridae to protect against brain infection. Neuron 100, 1527-1532 (2018).
35. C. Papadimitriou, H. Celikkaya, M. I. Cosacak, V. Mashkaryan, L. Bray, P. Bhattarai, K. Brandt, H. Hollak, X. Chen, S. He, C. L. Antos, W. Lin, A. K. Thomas, A. Dahl, T. Kurth, J. Friedrichs, Y. Zhang, U. Freudenberg, C. Werner, C. Kizil, 3D culture method for alzheimer's disease modeling reveals interleukin-4 rescues Aβ42-induced loss of human neural stem cell plasticity. Dev. Cell 46, 85-101.e8 (2018).
36. W. Bondareff, Age-related changes in brain extracellular space affect processing of amyloid-β peptides in Alzheimer's disease. J. Alzheimers Dis. 35, 1-6 (2013).
37. J. C. Carmichael, H. Yokota, R. C. Craven, A. Schmitt, J. W. Wills, The HSV-1 mechanisms of cell-to-cell spread and fusion are critically dependent on host PTP1B. PLOS Pathog. 14, e1007054 (2018).
38. M. Biancalana, S. Koide, Molecular mechanism of Thioflavin-T binding to amyloid fibrils. Biochim. Biophys. Acta 1804, 1405-1412 (2010).
39. J. E. Simpson, P. G. Ince, G. Lace, G. Forster, P. J. Shaw, F. Matthews, G. Savva, C. Brayne, S. B. Wharton; MRC Cognitive Function and Ageing Neuropathology Study Group, Astrocyte phenotype in relation to Alzheimer-type pathology in the ageing brain. Neurobiol. Aging 31, 578-590 (2010).
40. B. Decourt, D. K. Lahiri, M. N. Sabbagh, Targeting tumor necrosis factor alpha for Alzheimer's disease. Curr. Alzheimer Res. 14, 412-425 (2017).
41. G. M. Shankar, B. L. Bloodgood, M. Townsend, D. M. Walsh, D. J. Selkoe, B. L. Sabatini, Natural oligomers of the Alzheimer amyloid-β protein induce reversible synapse loss by modulating an NMDA-type glutamate receptor-dependent signaling pathway. J. Neurosci. 27, 2866-2875 (2007).
42. D. M. Walsh, I. Klyubin, J. V. Fadeeva, W. K. Cullen, R. Anwyl, M. S. Wolfe, M. J. Rowan, D. J. Selkoe, Naturally secreted oligomers of amyloid β protein potently inhibit hippocampal long-term potentiation in vivo. Nature 416, 535-539 (2002).
43. B. Y. Hu, J. P. Weick, J. Yu, L. X. Ma, X. Q. Zhang, J. A. Thomson, S. C. Zhang, Neural differentiation of human induced pluripotent stem cells follows developmental principles but with variable potency. Proc. Natl. Acad. Sci. U.S.A. 107, 4335-4340 (2010).
44. R. Patani, P. A. Lewis, D. Trabzuni, C. A. Puddifoot, D. J. A. Wyllie, R. Walker, C. Smith, G. E. Hardingham, M. Weale, J. Hardy, S. Chandran, M. Ryten, Investigating the utility of human embryonic stem cell-derived neurons to model ageing and neurodegenerative disease using whole-genome gene expression and splicing analysis. J. Neurochem. 122, 738-751 (2012).
45. J. Mertens, D. Reid, S. Lau, Y. Kim, F. H. Gage, Aging in a dish: iPSC-derived and directly induced neurons for studying brain aging and age-related neurodegenerative diseases. Annu. Rev. Genet. 52, 271-293 (2018).
46. A. LeBlanc, H. Liu, C. Goodyer, C. Bergeron, J. Hammond, Caspase-6 role in apoptosis of human neurons, amyloidogenesis, and Alzheimer's disease. J. Biol. Chem. 274, 23426-23436 (1999).
47. E. T. Lund, R. McKenna, D. B. Evans, S. K. Sharma, W. R. Mathews, Characterization of the in vitro phosphorylation of human tau by tau protein kinase II (cdk5/p20) using mass spectrometry. J. Neurochem. 76, 1221-1232 (2001).
48. J. Park, I. Wetzel, I. Marriott, D. Dreau, C. D'Avanzo, D. Y. Kim, R. E. Tanzi, H. Cho, A 3D human triculture system modeling neurodegeneration and neuroinflammation in Alzheimer's disease. Nat. Neurosci. 21, 941-951 (2018).
49. A. Zambrano, L. Solis, N. Salvadores, M. Cortes, R. Lerchundi, C. Otth, Neuronal cytoskeletal dynamic modification and neurodegeneration induced by infection with herpes simplex virus type 1. J. Alzheimers Dis. 14, 259-269 (2008).
50. M. A. Wozniak, A. L. Frost, R. F. Itzhaki, Alzheimer's disease-specific tau phosphorylation is induced by herpes simplex virus type 1. J. Alzheimers Dis. 16, 341-350 (2009).
51. M. A. Wozniak, R. F. Itzhaki, S. J. Shipley, C. B. Dobson, Herpes simplex virus infection causes cellular β-amyloid accumulation and secretase upregulation. Neurosci. Lett. 429, 95-100 (2007).
52. G. De Chiara, M. E. Marcocci, L. Civitelli, R. Argnani, R. Piacentini, C. Ripoli, R. Manservigi, C. Grassi, E. Garaci, A. T. Palamara, APP processing induced by herpes simplex virus type 1 (HSV-1) yields several APP fragments in human and rat neuronal cells. PLOS One 5, e13989 (2010).
53. S. Santana, M. Recuero, M. J. Bullido, F. Valdivieso, J. Aldudo, Herpes simplex virus type I induces the accumulation of intracellular β-amyloid in autophagic compartments and the inhibition of the non-amyloidogenic pathway in human neuroblastoma cells. Neurobiol. Aging 33, 430.e19-430.e33 (2012).
54. R. P. Friedrich, K. Tepper, R. Ronicke, M. Soom, M. Westermann, K. Reymann, C. Kaether, M. Fandrich, Mechanism of amyloid plaque formation suggests an intracellular basis of AP pathogenicity. Proc. Natl. Acad. Sci. U.S.A. 107, 1942-1947 (2010).
55. X. Hu, X. Zhou, W. He, J. Yang, W. Xiong, P. Wong, C. G. Wilson, R. Yan, BACE1 deficiency causes altered neuronal activity and neurodegeneration. J. Neurosci. 30, 8819-8829 (2010).
56. F. M. Laird, H. Cai, A. V. Savonenko, M. H. Farah, K. He, T. Melnikova, H. Wen, H. C. Chiang, G. Xu, V. E. Koliatsos, D. R. Borchelt, D. L. Price, H. K. Lee, P. C. Wong, BACE1, a major determinant of selective vulnerability of the brain to amyloid-β amyloidogenesis, is essential for cognitive, emotional, and synaptic functions. J. Neurosci. 25, 11693-11709 (2005).
57. M. H. Ou-Yang, J. E. Kurz, T. Nomura, J. Popovic, T. W. Rajapaksha, H. Dong, A. Contractor, D. M. Chetkovich, W. G. Tourtellotte, R. Vassar, Axonal organization defects in the hippocampus of adult conditional BACE1 knockout mice. Sci. Transl. Med. 10, eaao5620 (2018).
58. M. J. Savage, M. Iqbal, T. Loh, S. P. Trusko, R. Scott, R. Siman, Cathepsin G: Localization in human cerebral cortex and generation of amyloidogenic fragments from the β-amyloid precursor protein. Neuroscience 60, 607-619 (1994).
59. C. J. Holler, R. L. Webb, A. L. Laux, T. L. Beckett, D. M. Niedowicz, R. R. Ahmed, Y. Liu, C. R. Simmons, A. L. S. Dowling, A. Spinelli, M. Khurgel, S. Estus, E. Head, L. B. Hersh, M. P. Murphy, BACE2 expression increases in human neurodegenerative disease. Am. J. Pathol. 180, 337-350 (2012).
60. Z. Wang, Q. Xu, F. Cai, X. Liu, Y. Wu, W. Song, BACE2, a conditional β-secretase, contributes to Alzheimer's disease pathogenesis. JCI Insight 4, 123431 (2019).

### Example 2: Adaption of the Model for High Throughput Screening Assays

In the following Example, the inventors describe the adaption of their in vitro system of a human brain-like tissue model of herpes-induced Alzheimer's Disease (AD)[36] for use in high throughput screening assays. Specifically, the inventors propose to use this model to identify the following addition methods of screening.

Additional pathogens that mimic the AD phenotype induced by HSV-1.

The model described herein may be used for other pathogens beside HSV-1 (e.g., Pseudomonas gingivalis, human herpesvirus 6, etc.) which have been postulated to play a similar role in AD pathogenesis. The hiNSC model could be utilized to test the capacity of these pathogens to induce an AD-like phenotype in hiNSCs for additional studies.

Potential compounds that exacerbate the sporadic AD phenotype.

The model could be used to detect compounds in a screen for their ability to enhance the rate of development and/or increase the size of plaque-like formations (PLFs). This can be done by real-time monitoring of the in vitro model and the development or increase in one or more characteristic of AD-phenotype.

Potential compounds that prevent, cure or otherwise diminish AD phenotype.

The AD-model described in Example 1, and further comprising modified hiNSCs comprising one or more reporter gene could be used to assess compounds for their ability to reduce the rate of development and/or decrease the size of PLFs. The model could also be used to identify compounds with potential to treat AD post-generation. For example, large-scale screens could be performed using hiNSCs pre-treated with HSV-1 to induce a late-stage AD phenotype. Any compounds identified in these screens would be assessed for their ability to shrink, degrade, and/or digest pre-existing PLFs in these cultures.

Patient-specific responses to herpes-induced AD for personalized drug screening.

Somatic cells, such as dermal fibroblasts, can be harvested from patients and reprogrammed into hiNSCs. Certain genetic variants are more predisposed to developing AD relative to the general population—for example, people carrying the apolipoprotein E4 variant are at significantly increased risk for both herpes manifestation as well as AD. Generating patient-specific hiNSCs and subjecting them to herpes-induced AD will enable the identification of any individual differences in resulting AD-like phenotypes. These patient-specific hiNSCs can also be used in drug efficacy screens for personalized medicine.

Suitable methods for detecting, monitoring and assessing changes in the AD-phenotype are described in this Example. One or more of these methods can be used for monitoring AD-development, and or screening compounds or treatments in the sporadic AD-model described herein.

1) Fixation and Analysis of AD Phenotypes Using Immunostaining in Monolayer Cultures.

As described in Example 1, our herpes-induced model of AD can be reproducibly used to detect the following markers via immunostaining: markers of amyloid plaques (Amyloid Fibril, Abcam #ab201062), phosphorylated Tau (Phospho-Tau (Ser202, Thr205) (AT8), Thermofisher #MN1020), gliosis (glial fibrillary acidic protein (GFAP), Sigma #G9269) and apoptosis (Cleaved Caspase 3 (CC3), R&D #AF835). hiNSCs infected with HSV-1 develop AD-like phenotypes in less than one week. The resulting immunostaining images can be quantified using standard imaging software, such as ImageJ (NIH).

2) Fixation and Analysis of AD Phenotypes Using Immunostaining in 3D Cultures.

The herpes-induced AD system can also be incorporated into a 3D model using silk-based sponge scaffolds infused with collagen gels. This established 3D brain-like tissue model has the advantage of allowing for longer-term culture. This model can be used to mimic a low level HSV-1 infection characterized by the robust generation of beta amyloid plaques that persist in long-term cultures with neighboring neurons and glia. The herpes infection can be stopped with valacyclovir, and the 3D hiNSC cultures can continue to grow and be monitored over time.

3) Real-Time Assessments of AD Markers Using Specific Dyes and Labeling Systems.

Any suitable dye or labeling system can be used to track markers of AD in the 3D hiNSC cultures.

Thioflavin T dye can be used to track the development or treatment of amyloid plaques. HSV-1-infected hiNSCs display large plaque-like formations that stain positive with Thioflavin T dye (FIG. 10). In this example, fluorescence emitted by bound Thioflavin T dye (excitation emission 350 nm/440 nm) was monitored in hiNSCs that were mock-treated or infected with virus (HSV-1) once per hour over the course of 60 hours (FIG. 11). This produced a quantifiable sustained increase in percentage confluence of ThT fluorescence in the HSV-1-infected hiNSCs only, suggesting that this is a suitable method for quantifying amyloid plaque formation in real time. Notably, this dye can be modified to use in real time assays, such as an Incucyte Live-Cell Analysis System (Sartorius).

Lentivirus constructs that allow for stable expression of a fluorescently labeled nuclear protein can be used to quantify intact nuclei over time. Such lentivirus constructs are commercially available (e.g., Incucyte® NucLight Red Lentivirus Reagent, EF-1 Alpha Promoter, Puromycin selection, #4476). As part of their AD-like phenotype, HSV-1-infected hiNSCs exhibit enlargement, blebbing and fusion of nuclei, which ultimately results in the formation of multicellular plaques. Thus, the inventors envision that these constructs can be used to monitor and quantify abnormal and healthy nuclei in real time. In this case, low numbers of healthy normal-sized nuclei would be indicative of an AD-like phenotype.

4) Real-Time Assessments of AD Markers Using Genetically Modified hiNSCs.

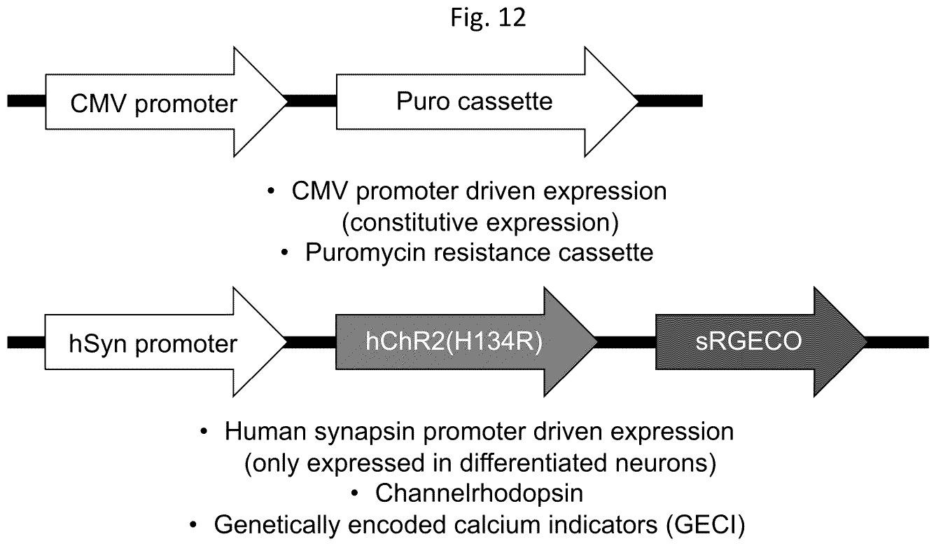

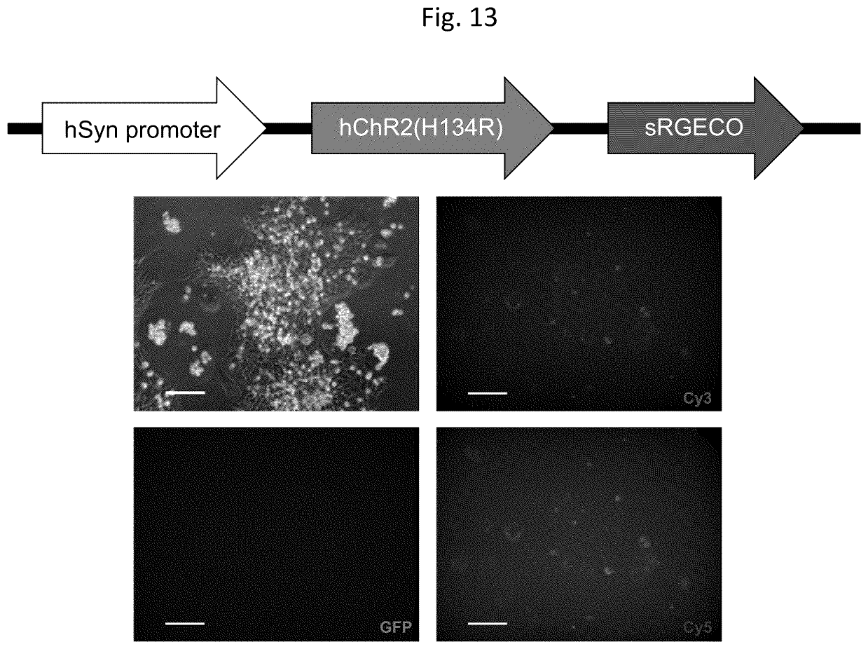

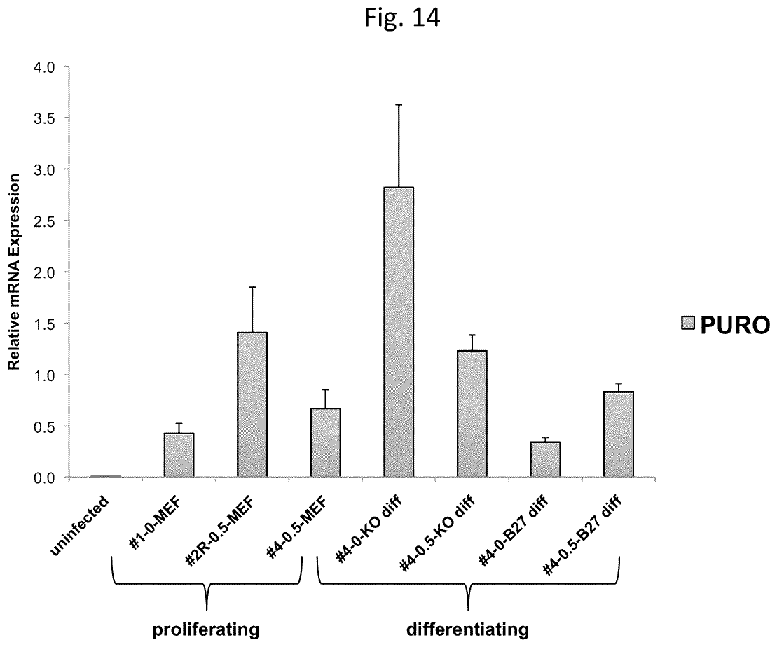

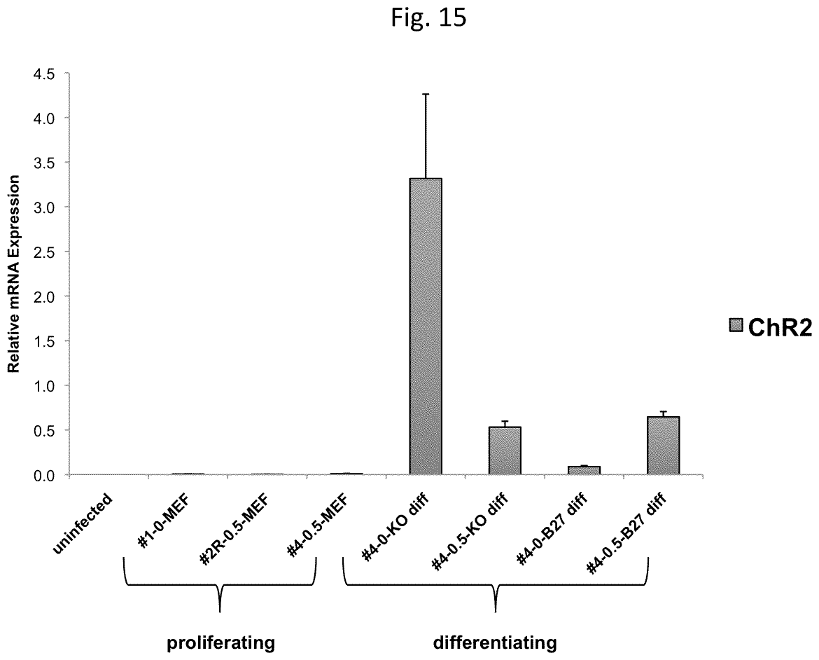

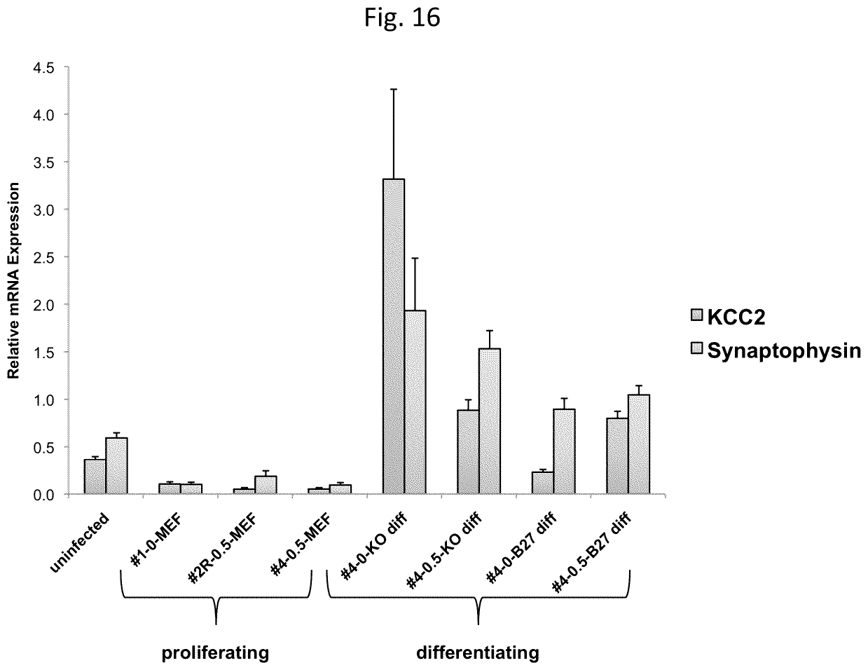

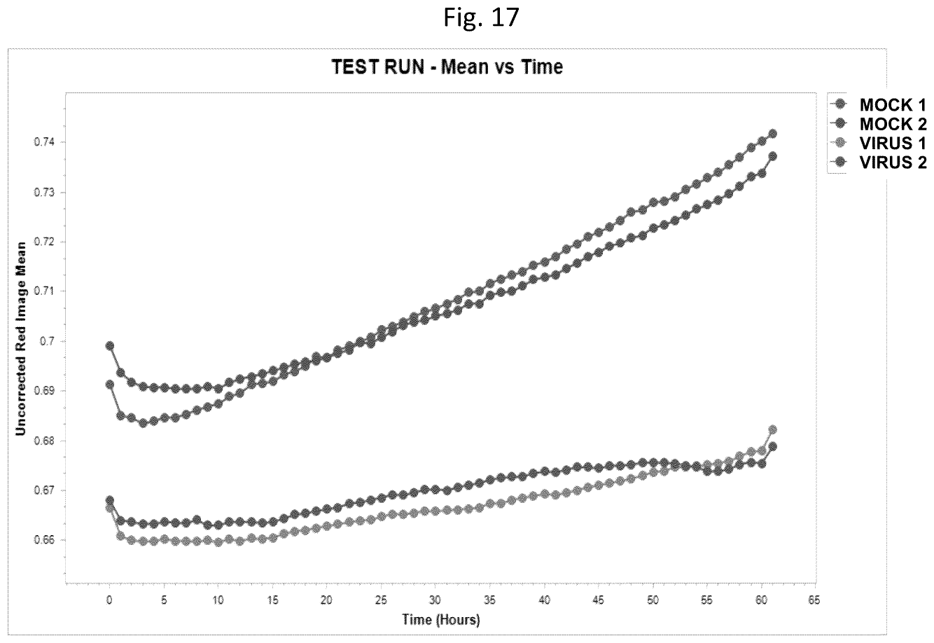

Lentiviral reporter constructs may be used to track the growth and function of healthy neurons in real time. The inventors have established a stable hiNSC line that allows for real-time assessment of AD phenotypes without the use of additional dyes or reagents. Reporter cells have also been developed to help monitor cell functions in real time during high throughput optical screening. Specifically, these reporter cells can be used to assess neuron stimulation, changes in healthy vs. AD-neurons, neuroinflammation, and other outcomes in screens to identify additional virus-induced effects and neurodegenerative disease phenotypes. This hiNSC line was generated via stable transduction with a lentiviral construct that expresses channelrhodopsin and a cherry-red calcium sensor. These genes are expressed under the control of a synapsin promoter such that they are only expressed in neurons that are fully differentiated and functioning properly. Channelrhodopsin may be used, for example, to image firing post-optostimulation in healthy vs. AD neurons. The construct comprises a puromycin cassette, which is driven by a constitutively active CMV promoter, allows for stable selection of cells with this construct at the proliferative hiNSC stage (FIG. 12). Upon differentiation, the stably transduced hiNSCs express a cherry red signal only in mature, healthy neurons (FIG. 13). The puromycin resistance cassette is expressed in hiNSC lines regardless of differentiation status, as demonstrated by qPCR analysis (FIG. 14). The expression of channelrhodopsin is only observable by qPCR in hiNSC lines that have been induced to differentiate, demonstrating the specificity of the synapsin promoter for mature neurons (FIG. 15). An increase in mature neuron-specific markers, i.e., potassium-chloride cotransporter 2 (KCC2) and synaptophysin, in the hiNSC lines (based on qPCR analysis) is correlated with synapsin promoter induced expression of channelrhodopsin (FIG. 16). The IncuCyte® Live Cell Analysis System (Sartorius) was used to image hiNSCs stably expressing this polycistronic optogenetic reporter construct in real time (FIG. 17). These genetically modified hiNSCs were subjected to mock or viral (HSV-1) infection, and were then imaged once per hour over the course of 60 hours. Mock-infected hiNSCs showed steady increase in total red fluorescence, whereas virus-infected hiNSCs did not, reflecting a lack of healthy, functioning neurons in HSV-1-infected AD-like cultures.

Lentiviral reporter construct to track gliosis in real time. Commercially available constructs may be used to track gliosis in the hiNSCs in real time. For example, constructs that include a glial fibrillary acidic protein (GFAP) promoter (e.g., the adenoviral construct, pAAV-GFAP-hChR2(H134R)-mCherry) can be used to specifically label only those cells that become glia (i.e., express GFAP). As part of their AD-like phenotype, HSV-1-infected hiNSCs exhibit robust gliosis. Thus, the inventors envision that these constructs can be used to label and quantify cells that upregulate GFAP, as indicated by cherry red expression.

### Discussion:

A number of in vitro human models of AD have been previously developed, including several that rely on human induced pluripotent stem cell (hiPSC) technology. One benefit of such methods is that the hiPSCs can be generated from a patient's cells (e.g., a patient with a known disorders and/or genetic mutation), allowing them to serve as a personalized “disease in a dish”. However, while protocols for differentiating iPSCs into various neuronal phenotypes have been developed, these protocols are time consuming, require multiple complicated intermediate steps (e.g., the formation of non-adherent embryoid bodies and neurospheres), and produce cells with that have undergone varying degrees of neuronal differentiation[37]. A number of studies have been conducted using cells derived from patients with familial EOAD, which is associated with mutations in the genes APP, PSEN1 and PSEN2[2]. Because these genetic mutations provide the stimulus for initiating AD in such models, these studies do not accurately model disease onset or progression in the majority of AD cases, which develop sporadically. Furthermore, because the process by which somatic cells are converted into iPSCs is thought to epigenetically “reset” the cells into an embryonic-like state, it has been suggested that these cell lines lack an appropriate aging profile upon reprogramming. For example, iPSC-derived neurons have been reported to have an immature phenotype and lack aging markers [38], which is not ideal for modeling a neurodegenerative disease in which the primary risk factor is age. It has been proposed that direct reprogramming or transdifferentiation methods, which bypass the pluripotent state, may better maintain an aging signature in resulting neurons[39].

In other studies, AD-like microenvironments have been created synthetically. While some of these synthetic models have shown certain characteristics of AD, such as neuronal loss[40] or Tau phosphorylation[41], the majority of these models are often unable to fully recapitulate the more complex features of AD, such as plaque formation, reactive gliosis, and loss of neural tissue functionality. Further, many of these models require months of culture to establish a discernible phenotype [33]. Importantly, most if not all of these studies require the use of known AD mediators to initiate an AD-like state. For example, in some studies, AD was induced in the model system using viruses that overexpress AD mediators such as PSEN1 [33], pro-inflammatory cytokines [42], and/or detrimental Aβ peptides [33, 35, 42]. Models that rely on such exogenous mediators to artificially induce plaque formation are not reflective of the in vivo condition.

There is increasing evidence to suggest a clinical correlation between pathogens such as herpes and the development of sporadic AD[20, 21] including the findings that HSV-1 DNA was detected in AD patient brain tissue[24-26] and recent epidemiological studies even reporting HSV-1 infection as a significant risk factor later AD onset[27]. While there is no published correlative evidence of active Herpes infections in AD patients, these clinical findings suggest that active infection may not be a requirement for AD, but rather that prior history of Herpes infection is sufficient to initiate AD pathogenesis.

Importantly, we are not the first to describe an experimental link between HSV-1 and AD. Several reports have described in vitro monolayer cell culture models in which HSV-1 has led to neuronal death[32, 43], Tau phosphorylation[44], and intracellular expression of various isoforms of APP cleavage products[45-47]. While the finding that certain proteins known to be involved in the amyloidogenic pathway are also upregulated in response to HSV-1 infection is exciting, few studies have reported the formation of multicellular plaque-like structures in neuron cultures. These PLFs are composed of multiple merged infected hiNSCs, averaging ˜19 nuclei per individual PLF (data not shown). A recent study suggested an intracellular basis for the formation of these extracellular Aβ plaques[48]. Briefly, small Aβ oligomers form and get transported to vesicular bodies, after which a phagocytic event occurs to generate multicellular plaques that apoptose, releasing their contents into the extracellular space. It is plausible that similar mechanisms occur in our HSV-mediated in vitro system, as the PLFs formed in these cultures are multicellular and apoptotic.

Furthermore, most of these HSV-AD studies have relied on the use of primary cells derived from rodent models[43, 46] and/or human cancer cell lines[44-47], and often require the use of exogenous AD mediators[45, 47]. Recent work by Eimer et al.1[34] reported that Aβ proteins sequestered herpes viruses in 3D human neural cell cultures using an immortalized cell line. However, this study required the use of lentiviruses overexpressing human β-amyloid precursor protein (APP) or both APP and presenilin 1 (PSEN1), containing familial AD mutations, which is not truly physiologically reflective of sporadic AD pathogenesis. In addition, this model did not demonstrate multiple aspects of AD physiology including multicellular plaque formation, reactive gliosis or loss of brain tissue functionality.

Thus, the human brain-like tissue models of AD of the present invention offers several significant advantages over other available models of AD. First, the model disclosed herein encompasses many facets of human AD, including Aβ plaque formation, neuronal loss, reactive gliosis, neuroinflammation, and diminished neural network functionality. Importantly, the present model achieves this robust AD phenotype in a short time relative to other 3D model systems of AD[33]. Second, the model requires only unmodified, wild type cells as input, and it functions in the absence of any exogenous factors specifically known to regulate or induce AD[33-35]. Thus, this model of herpes-induced AD will enable future studies that aim to elucidate the mechanisms of HSV-1 mediated AD pathogenesis and to identify potential downstream targets for treating this devastating disease.

### References for Example 2

1. 2016 Alzheimer's disease facts and figures. Alzheimers Dement, 2016. 12(4): p. 459-509.
2. Naj, A. C. and G. D. Schellenberg, Genomic variants, genes, and pathways of Alzheimer's disease: An overview. Am J Med Genet B Neuropsychiatr Genet, 2017. 174(1): p. 5-26.
3. Weyer, S. W., et al., APP and APLP2 are essential at PNS and CNS synapses for transmission, spatial learning and LTP. EMBO J, 2011. 30(11): p. 2266-80.
4. Kelleher, R. J., 3rd and J. Shen, Presenilin-1 mutations and Alzheimer's disease. Proc Natl Acad Sci USA, 2017. 114(4): p. 629-631.
5. De Strooper, B., R. Vassar, and T. Golde, The secretases: enzymes with therapeutic potential in Alzheimer disease. Nat Rev Neurol, 2010. 6(2): p. 99-107.
6. Takahashi, R. H., T. Nagao, and G. K. Gouras, Plaque formation and the intraneuronal accumulation of beta-amyloid in Alzheimer's disease. Pathol Int, 2017. 67(4): p. 185-193.
7. Perez-Nievas, B. G. and A. Serrano-Pozo, Deciphering the Astrocyte Reaction in Alzheimer's Disease. Front Aging Neurosci, 2018. 10: p. 114.
8. Ho, G. J., et al., Mechanisms of cell signaling and inflammation in Alzheimer's disease. Curr Drug Targets Inflamm Allergy, 2005. 4(2): p. 247-56.
9. Reddy, P. H., et al., Differential loss of synaptic proteins in Alzheimer's disease: implications for synaptic dysfunction. J Alzheimers Dis, 2005. 7(2): p. 103-17; discussion 173-80.
10. Arber, C., C. Lovejoy, and S. Wray, Stem cell models of Alzheimer's disease: progress and challenges. Alzheimers Res Ther, 2017. 9(1): p. 42.
11. Balin, B. J., et al., Chlamydophila pneumoniae and the etiology of late-onset Alzheimer's disease. J Alzheimers Dis, 2008. 13(4): p. 371-80.
12. Miklossy, J., Emerging roles of pathogens in Alzheimer disease. Expert Rev Mol Med, 2011.13:p.e30.
13. Itzhaki, R. F., et al., Microbes and Alzheimer's Disease. J Alzheimers Dis, 2016. 51(4): p. 979-84.
14. Harris, S. A. and E. A. Harris, Herpes Simplex Virus Type 1 and Other Pathogens are Key Causative Factors in Sporadic Alzheimer's Disease. J Alzheimers Dis, 2015. 48(2): p. 319-53.
15. Readhead, B., et al., Multiscale Analysis of Independent Alzheimer's Cohorts Finds Disruption of Molecular, Genetic, and Clinical Networks by Human Herpesvirus. Neuron, 2018. 99(1): p. 64-82 e7.
16. Giuffrida, M. L., et al., Beta-amyloid monomers are neuroprotective. J Neurosci, 2009. 29(34): p. 10582-7.
17. Miklossy, J., Alzheimer's disease—a neurospirochetosis. Analysis of the evidence following Koch's and Hill's criteria. J Neuroinflammation, 2011. 8: p. 90.
18. Pisa, D., et al., Direct visualization of fungal infection in brains from patients with Alzheimer's disease. J Alzheimers Dis, 2015. 43(2): p. 613-24.
19. Bates, K. A., et al., Chronic gliosis triggers Alzheimer's disease-like processing of amyloid precursor protein. Neuroscience, 2002. 113(4): p. 785-96.
20. Itzhaki, R. F., Corroboration of a Major Role for Herpes Simplex Virus Type 1 in Alzheimer's Disease. Front Aging Neurosci, 2018. 10: p. 324.
21. Itzhaki, R. F., Herpes simplex virus type 1 and Alzheimer's disease: possible mechanisms and signposts. FASEB J, 2017. 31(8): p. 3216-3226.
22. Liu, H., et al., Mechanisms of Blood-Brain Barrier Disruption in Herpes Simplex Encephalitis. J Neuroimmune Pharmacol, 2019. 14(2): p. 157-172.
23. Ball, M. J., “Limbic predilection in Alzheimer dementia: is reactivated herpesvirus involved?”. Can J Neurol Sci, 1982. 9(3): p. 303-6.
24. Jamieson, G. A., et al., Latent herpes simplex virus type 1 in normal and Alzheimer's disease brains. J Med Virol, 1991. 33(4): p. 224-7.
25. Jamieson, G. A., et al., Herpes simplex virus type 1 DNA is present in specific regions of brain from aged people with and without senile dementia of the Alzheimer type. J Pathol, 1992. 167(4): p. 365-8.
26. Wozniak, M. A., A. P. Mee, and R. F. Itzhaki, Herpes simplex virus type 1 DNA is located within Alzheimer's disease amyloid plaques. J Pathol, 2009. 217(1): p. 131-8.
27. Itzhaki, R. F. and R. Lathe, Herpes Viruses and Senile Dementia: First Population Evidence for a Causal Link. J Alzheimers Dis, 2018. 64(2): p. 363-366.
28. Cairns, D. M., et al., Expandable and Rapidly Differentiating Human Induced Neural Stem Cell Lines for Multiple Tissue Engineering Applications. Stem Cell Reports, 2016. 7(3): p. 557-70.
29. Cairns, D. M., et al., Niclosamide rescues microcephaly in a humanized in vivo model of Zika infection using human induced neural stem cells. Biol Open, 2018. 7(1).
30. Chwalek, K., et al., In vitro bioengineered model of cortical brain tissue. Nat Protoc, 2015. 10(9): p. 1362-1373.
31. Wozniak, M. A., et al., Antivirals reduce the formation of key Alzheimer's disease molecules in cell cultures acutely infected with herpes simplex virus type 1. PLoS One, 2011. 6(10): p. e25152.
32. Bourgade, K., et al., Protective Effect of Amyloid-beta Peptides Against Herpes Simplex Virus-1 Infection in a Neuronal Cell Culture Model. J Alzheimers Dis, 2016. 50(4): p. 1227-41.
33. Choi, S. H., et al., A three-dimensional human neural cell culture model of Alzheimer's disease. Nature, 2014. 515(7526): p. 274-8.
34. Eimer, W. A., et al., Alzheimer's Disease-Associated beta-Amyloid Is Rapidly Seeded by Herpesviridae to Protect against Brain Infection. Neuron, 2018. 100(6): p. 1527-1532.
35. Papadimitriou, C., et al., 3D Culture Method for Alzheimer's Disease Modeling Reveals Interleukin-4 Rescues Abeta42-Induced Loss of Human Neural Stem Cell Plasticity. Dev Cell, 2018. 46(1): p. 85-101 e8.
36. Cairns, D. M., et al., A 3D human brain,Äílike tissue model of herpes-induced Alzheimer,Äôs disease. Science Advances, 2020. 6(19): p. eaay8828.
37. Hu, B. Y., et al., Neural differentiation of human induced pluripotent stem cells follows developmental principles but with variable potency. Proc Natl Acad Sci USA, 2010. 107(9): p. 4335-40.
38. Patani, R., et al., Investigating the utility of human embryonic stem cell-derived neurons to model ageing and neurodegenerative disease using whole-genome gene expression and splicing analysis. J Neurochem, 2012. 122(4): p. 738-51.
39. Mertens, J., et al., Aging in a Dish: iPSC-Derived and Directly Induced Neurons for Studying Brain Aging and Age-Related Neurodegenerative Diseases. Annu Rev Genet, 2018. 52: p. 271-293.
40. LeBlanc, A., et al., Caspase-6 role in apoptosis of human neurons, amyloidogenesis, and Alzheimer's disease. J Biol Chem, 1999. 274(33): p. 23426-36.
41. Lund, E. T., et al., Characterization of the in vitro phosphorylation of human tau by tau protein kinase II (cdk5/p20) using mass spectrometry. J Neurochem, 2001. 76(4): p. 1221-32.
42. Park, J., et al., A 3D human triculture system modeling neurodegeneration and neuroinflammation in Alzheimer's disease. Nat Neurosci, 2018. 21(7): p. 941-951.
43. Zambrano, A., et al., Neuronal cytoskeletal dynamic modification and neurodegeneration induced by infection with herpes simplex virus type 1. J Alzheimers Dis, 2008. 14(3): p. 259-69.
44. Wozniak, M. A., A. L. Frost, and R. F. Itzhaki, Alzheimer's disease-specific tau phosphorylation is induced by herpes simplex virus type 1. J Alzheimers Dis, 2009. 16(2): p. 341-50.
45. Wozniak, M. A., et al., Herpes simplex virus infection causes cellular beta-amyloid accumulation and secretase upregulation. Neurosci Lett, 2007. 429(2-3): p. 95-100.
46. De Chiara, G., et al., APP processing induced by herpes simplex virus type 1 (HSV-1) yields several APP fragments in human and rat neuronal cells. PLoS One, 2010.5(11): p. e13989.
47. Santana, S., et al., Herpes simplex virus type I induces the accumulation of intracellular beta-amyloid in autophagic compartments and the inhibition of the non-amyloidogenic pathway in human neuroblastoma cells. Neurobiol Aging, 2012. 33(2): p. 430 e19-33.
48. Friedrich, R. P., et al., Mechanism of amyloid plaque formation suggests an intracellular basis of Abeta pathogenicity. Proc Natl Acad Sci USA, 2010. 107(5): p. 1942-7.

### Example 3: Real-Time Functional Readout of a 3D Brain-Like Tissue Model of Herpes-Induced AD

### Results

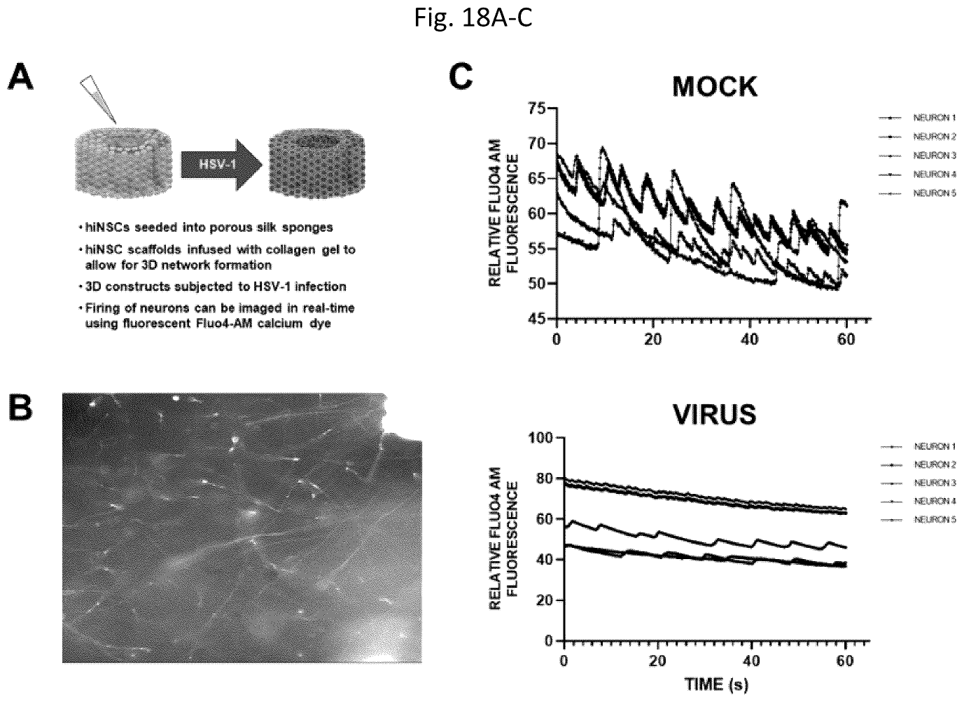

Assessment of functionality is the gold standard of in vitro model systems. While in our recent publication Cairns et. al. (“A 3D human brain-like tissue model of herpes-induced Alzheimer's disease” Sci Adv. 2020 May 6; 6(19), incorporated herein by reference), we demonstrated quantifiable decrease in action potentials as evidenced by electrophysiological recording (local field potential) (FIG. 18), this method requires complex equipment and expertise. Neuronal firing can also be measured and quantified using fluorescent dyes such as Fluo4-AM, to image intracellular calcium influx in real-time, without the need for high tech electrophysiology rigs and trained personnel.

Example of a Positive Result from High Throughput Drug Screening for AD Therapeutics.

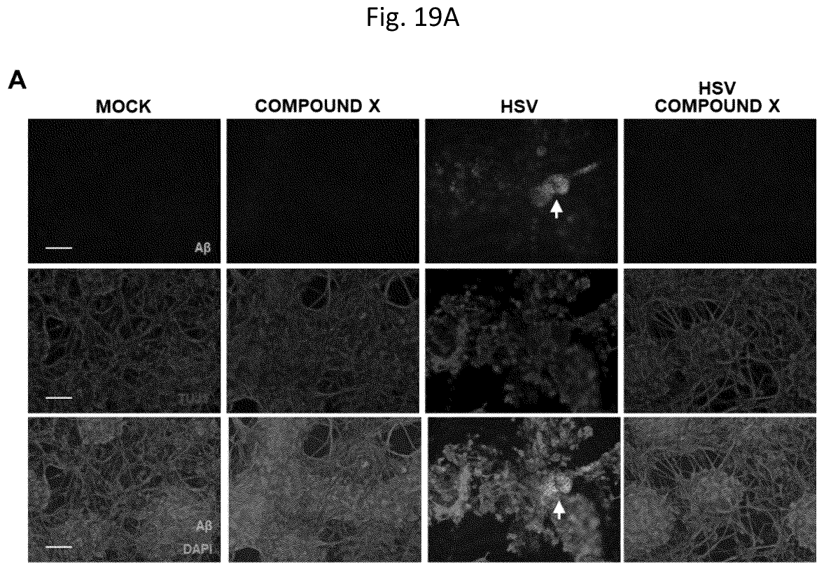

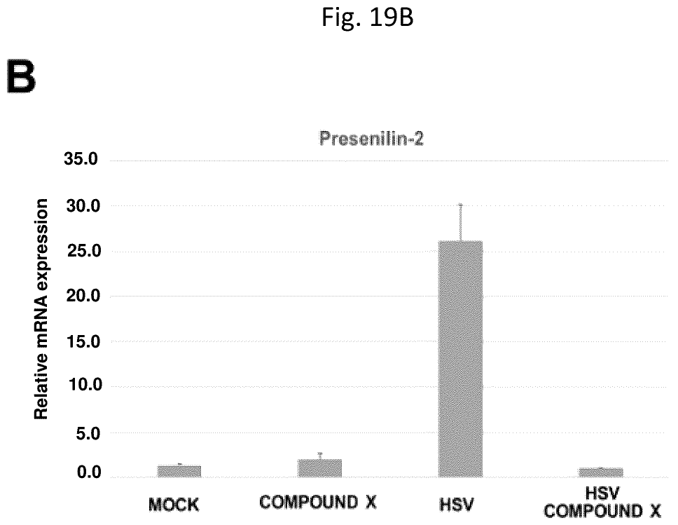

Here as proof of concept we show the results from a successful AD drug candidate screen. Briefly, hiNSCs were grown as monolayer cultures for 4 days before being subjected to MOCK or HSV-1 infection and/or drug treatment for 4 days. Results demonstrate that HSV-1 treatment induced robust beta amyloid-positive plaque formations (PLFs), as previously reported. Importantly, treatment with “COMPOUND X” prevented herpes-induced formation of PLFs and presenilin-2 (PSEN2) upregulation, suggesting that this compound is a good candidate for follow-up studies to further assess anti-AD efficacy (FIG. 19).

Generation of a Stable hiNSC Reporter Line for Detecting AD-Like Phenotypes in Real-Time

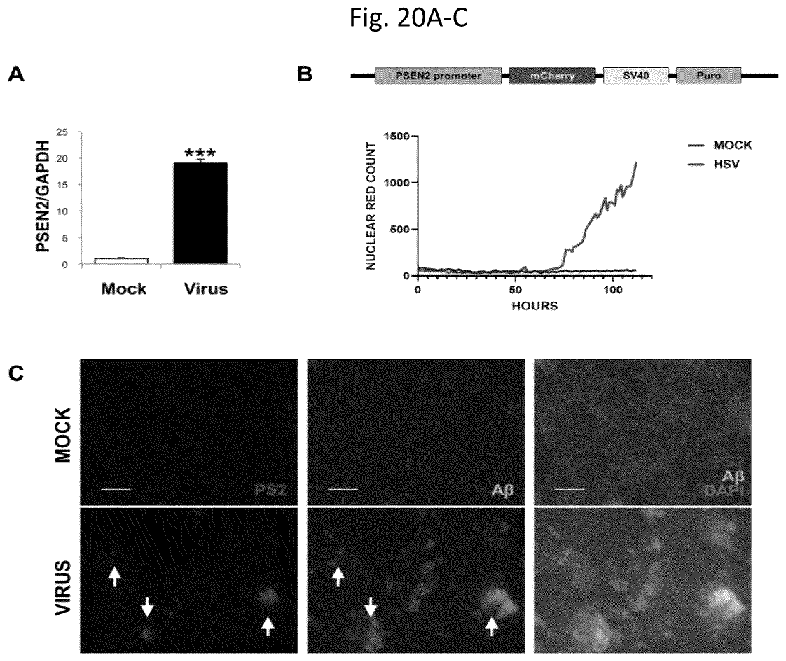

While the herpes-induced AD model described herein is high throughput in that multiple drugs can be tested simultaneously and downstream readouts such as immunostaining and qPCR can be performed in approximately one week; optimizing this system for use with a real-time reporter would even further streamline these assays for AD drug discovery. Here we report the generation of a stable hiNSC reporter line for detecting AD-like phenotypes in real-time (FIG. 20). Briefly, the human presenilin 2 (PSEN2) promoter was cloned upstream of an mCherry reporter in a lentiviral backbone. Upon herpes-mediated induction of PSEN2, mCherry is expressed. This reporter also has a puromycin cassette to allow for stable selection (FIG. 20B). mCherry fluorescence was significantly higher in cells infected with herpesvirus simplex 1 (HSV-1) compared to mock infected cells (FIGS. 20A and C).

### Induction of HSV-1 Latency for Discovery of Triggers of Viral Reactivation

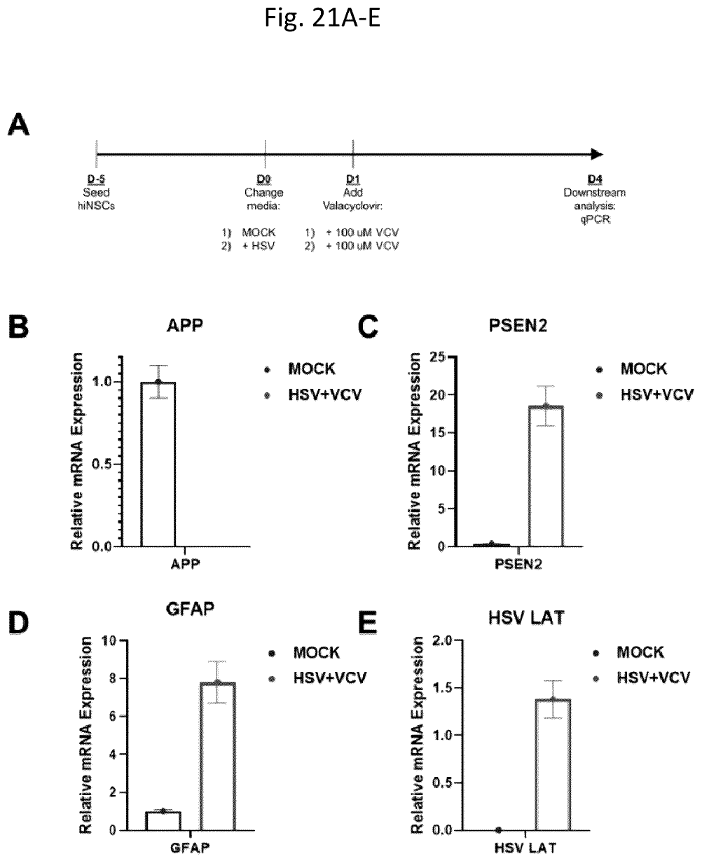

HSV-1 typically lies latent in the peripheral nervous system indefinitely, and it is only upon reactivation that this virus can exert its detrimental effects such as cold sores and potentially neurodegeneration. Understanding potential triggers of this viral reactivation are crucial in elucidating downstream pathways involved in disease pathogenesis. There are currently few if any suitable in vitro models of HSV-1 latency. Here, we describe a method with which to induce latency of HSV-1 in vitro. The addition of the antiviral drug valacyclovir was associated with a significant increase in both PSEN2 expression as well as HSV-1 latency associated transcript (LAT) (FIGS. 21C and E). Induction of latency with valacyclovir was also associated with an increase in glial fibrillary acidic protein (GFAP) expression (FIG. 21D), which is indicative of gliosis.

### Discussion

In summary, the disclosed working example 3 demonstrates the effectiveness of using the instantly disclosed methods and compositions for high-throughput screening of drugs effective for the treatment of Alzheimer's disease (AD).

SEQUENCES:

SEQ ID NO: 1
Human synapsin I promoter:
GTGTCTAGACTGCAGAGGGCCCTGCGTATGAGTGCAAGTGGGTTTTAGGACCAGGATGAGGCGGGGT
GGGGGTGCCTACCTGACGACCGACCCCGACCCACTGGACAAGCACCCAACCCCCATTCCCCAAATTG
CGCATCCCCTATCAGAGAGGGGGAGGGGAAACAGGATGCGGCGAGGCGCGTGCGCACTGCCAGCTTC
AGCACCGCGGACAGTGCCTTCGCCCCCGCCTGGCGGCGCGCGCCACCGCCGCCTCAGCACTGAAGGC
GCGCTGACGTCACTCGCCGGTCCCCCGCAAACTCCCCTTCCCGGCCACCTTGGTCGCGTCCGCGCCG
CCGCCGGCCCAGCCGGACCGCACCACGCGAGGCGCGAGATAGGGGGGCACGGGCGCGACCATCTGCG
CTGCGGCGCCGGCGACTCAGCGCTGCCTCAGTCTGCGGTGGGCAGCGGAGGAGTCGTGTCGTGCCTG
AGAGCGCAGTCGAGAA
SEQ ID NO: 2
Genetically encoded Ca2+ indicator rGECO:
GCGGGAGTCTGGTGAGCAAGGGCGAGGAGGATAACATGGCCATCATCAAGGAGTTCATGCGCTTCAA
GGTGCACATGGAGGGCTCCGTGAACGGCCACGAGTTCGAGATCGAGGGCGAGGGCGAGGGCCGCCCC
TACGAGGCCTTTCAGACCGCTAAGCTGAAGGTGACCAAGGGTGGCCCCCTGCCCTTCGCCTGGGACA
TCCTGTCCCCTCAGTTCATGTACGGCTCCAAGGCCTACATTAAGCACCCAGCCGACATCCCCGACTA
CTTCAAGCTGTCCTTCCCCGAGGGCTTCAGGTGGGACCGCGTGATGAACTTCGAGGACGGCGGCATT
ATTCACGTGAACCAGGACTCCTCCCTGCAGGACGGCGTATTCATCTACAAGGTGAAGCTGCGCGGCA
CCAACTTCCCCCCCGACGGCCCCGTAATGCAGAAGAAGACCATGGGCTGGGAGGCTACGCGTGACGA
CCTGACTGAAGAGCAGATCGCAGAATTTAAAGAGGCTTTCTCCCTATTTGACAAGGACGGGGATGGG
ACGATAACAACCAAGGAGCTTGGGACGGTGTTCCGATCGCTGGGGCAGAACCCCACAGAAGCAGAGC
TGCAGGACATGATCAATGAAGTAGATGCCGACGGTGACGGCACATTCGACTTCCCTGAGTTCCTGAC
GATGATGGCAAGAAAAATGAATGACACAGACAGTGAAGAGGAAATTCGCGAAGCGTTCCGCGTGTTT
GATAAGGACGGCAATGGCTACATCGGCGCAGCAGAGCTTCGCCACGTGATGACAGACCTTGGAGAGA
AGTTAACAGATGAGGAGGTTGATGAAATGATCAGGGTAGCAGACATCGATGGGGATGGTCAGGTAAA
CTACGAAGAGTTTGTACAAATGATGACAGCGAAGTAG
SEQ ID NO: 3
Glial fibrillary acidic protein (GFAP) promoter:
CCCACCTCCCTCTCTGTGCTGGGACTCACAGAGGGAGACCTCAGGAGGCAGTCTGTCCATCACATGT
CCAAATGCAGAGCATACCCTGGGCTGGGCGCAGTGGCGCACAACTGTAATTCCAGCACTTTGGGAGG
CTGATGTGGAAGGATCACTTGAGCCCAGAAGTTCTAGACCAGCCTGGGCAACATGGCAAGACCCTAT
CTCTACAAAAAAAGTTAAAAAATCAGCCACGTGTGGTGACACACACCTGTAGTCCCAGCTATTCAGG
AGGCTGAGGTGAGGGGATCACTTAAGGCTGGGAGGTTGAGGCTGCAGTGAGTCGTGGTTGCGCCACT
GCACTCCAGCCTGGGCAACAGTGAGACCCTGTCTCAAAAGACAAAAAAAAAAAAAAAAAAAAAAAGA
ACATATCCTGGTGTGGAGTAGGGGACGCTGCTCTGACAGAGGCTCGGGGGCCTGAGCTGGCTCTGTG
AGCTGGGGAGGAGGCAGACAGCCAGGCCTTGTCTGCAAGCAGACCTGGCAGCATTGGGCTGGCCGCC
CCCCAGGGCCTCCTCTTCATGCCCAGTGAATGACTCACCTTGGCACAGACACAATGTTCGGGGTGGG
CACAGTGCCTGCTTCCCGCCGCACCCCAGCCCCCCTCAAATGCCTTCCGAGAAGCCCATTGAGCAGG
GGGCTTGCATTGCACCCCAGCCTGACAGCCTGGCATCTTGGGATAAAAGCAGCACAGCCCCCTAGGG
GCTGCCCTTGCTGTGTGGCGCCACCGGCGGTGGAGAACAAGGCTCTATTCAGCCTGTGCCCAGGAAA
GGGGATCAGGGGATGCCCAGGCATGGACAGTGGGTGGCAGGGGGGGAGAGGAGGGCTGTCTGCTTCC
CAGAAGTCCAAGGACACAAATGGGTGAGGGGACTGGGCAGGGTTCTGACCCTGTGGGACCAGAGTGG
AGGGCGTAGATGGACCTGAAGTCTCCAGGGACAACAGGGCCCAGGTCTCAGGCTCCTAGTTGGGCCC
AGTGGCTCCAGCGTTTCCAAACCCATCCATCCCCAGAGGTTCTTCCCATCTCTCCAGGCTGATGTGT
GGGAACTCGAGGAAATAAATCTCCAGTGGGAGACGGAGGGGTGGCCAGGGAAACGGGGCGCTGCAGG
AATAAAGACGAGCCAGCACAGCCAGCTCATGTGTAACGGCTTTGTGGAGCTGTCAAGGCCTGGTCTC
TGGGAGAGAGGCACAGGGAGGCCAGACAAGGAAGGGGTGACCTGGAGGGACAGATCCAGGGGCTAAA
GTCCTGATAAGGCAAGAGAGTGCCGGCCCCCTCTTGCCCTATCAGGACCTCCACTGCCACATAGAGG
CCATGATTGACCCTTAGACAAAGGGCTGGTGTCCAATCCCAGCCCCCAGCCCCAGAACTCCAGGGAA
TGAATGGGCAGAGAGCAGGAATGTGGGACATCTGTGTTCAAGGGAAGGACTCCAGGAGTCTGCTGGG
AATGAGGCCTAGTAGGAAATGAGGTGGCCCTTGAGGGTACAGAACAGGTTCATTCTTCGCCAAATTC
CCAGCACCTTGCAGGCACTTACAGCTGAGTGAGATAATGCCTGGGTTATGAAATCAAAAAGTTGGAA
AGCAGGTCAGAGGTCATCTGGTACAGCCCTTCCTTCCCTTTTTTTTTTTTTTTTTTTGTGAGACAAG
GTCTCTCTCTGTTGCCCAGGCTGGAGTGGCGCAAACACAGCTCACTGCAGCCTCAACCTACTGGGCT
CAAGCAATCCTCCAGCCTCAGCCTCCCAAAGTGCTGGGATTACAAGCATGAGCCACCCCACTCAGCC
CTTTCCTTCCTTTTTAATTGATGCATAATAATTGTAAGTATTCATCATGGTCCAACCAACCCTTTCT
TGACCCACCTTCCTAGAGAGAGGGTCCTCTTGCTTCAGCGGTCAGGGCCCCAGACCCATGGTCTGGC
TCCAGGTACCACCTGCCTCATGCAGGAGTTGGCGTGCCCAGGAAGCTCTGCCTCTGGGCACAGTGAC
CTCAGTGGGGTGAGGGGAGCTCTCCCCATAGCTGGGCTGCGGCCCAACCCCACCCCCTCAGGCTATG
CCAGGGGGTGTTGCCAGGGGCACCCGGGCATCGCCAGTCTAGCCCACTCCTTCATAAAGCCCTCGCA
TCCCAGGAGCGAGCAGAGCCAGAGCAGGTTGGAGAGGAGACGCATCACCTCCGCTGCTCGCCGGG

1. An in vitro model of Alzheimer's disease (AD) comprising a population of human induced neural stem cells (hiNSCs) infected with a low multiplicity of infection of herpes simplex virus type 1 (HSV-1).

2. The in vitro model of AD of claim 1, wherein the hiNSCs are reprogrammed from somatic cells from a subject.

3. The in vitro model of AD of claim 1, wherein the HSV-1 infected hiNSCs exhibit
(a) large, multicellular, dense Aβ+ fibrillar plaque-like formations (PLFs);
(b) expression of PSEN1 and PSEN2 at higher levels as compared to non-infected control cells;
(c) reactive gliosis;
(d) one or more indicators of neuroinflammation; or
(e) one or more of (a)-(d).

4. The in vitro model of AD of claim 1, wherein the hiNSCs are genetically modified to stably express a reporter gene under the control of a synapsin promoter, wherein expression of the reporter gene indicates that hiNSCs have differentiated into mature, functioning neurons.

5. The in vitro model of AD of claim 4, wherein the reporter gene is a fluorescently tagged calcium sensor.

6. The in vitro model of AD of claim 4, wherein the hiNSCs are genetically modified to further express channelrhodopsin.

7. The in vitro model of AD of claim 1, wherein the hiNSCs are genetically modified to further express a second reporter gene under the control of a glial fibrillary acidic protein (GFAP) promoter, a tumor necrosis factor (TNF)-α promoter, or a presenilin-2 (PSEN2) promoter.

8. The in vitro model of AD of claim 1, wherein the hiNSCs are grown in a monolayer culture.

9. The in vitro model of claim 1, wherein the hiNSCs are grown in a three-dimensional biomaterial-based scaffold.

10. The in vitro model of claim 9, wherein the three-dimensional biomaterial-based scaffold comprises a silk-based sponge scaffold infused with collagen gel.

11. The in vitro model of claim 2, wherein the somatic cells are from a patient diagnosed with AD or at risk of developing AD.

12. A method of generating an in vitro model of Alzheimer's disease (AD), the method comprising:
a) infecting a population of human induced neuronal stem cells (hiNSCs) with a low multiplicity of infection of herpes simplex virus-1 (HSV-1) to produce a population of herpes-infected hiNSCs, and
b) culturing the herpes-infected hiNSCs in culture medium for at least one day, preferably three days,
wherein the hiNSCs develop an AD-like phenotype.

13. The method of claim 12, wherein the hiNSCs are contacted with the HSV-1 at a multiplicity of infection of 0.001 or less.

14. The method of claim 12, wherein the method further comprises seeding hiNSCs into a biomaterial-based scaffold after step (a).

15. The method of claim 14, wherein the biomaterial-based scaffold comprises a silk-based sponge scaffold infused with collagen gel.

16. (canceled)

17. The method of claim 12, wherein, prior to step (a), the method further comprises:
genetically modifying the hiNSCs by contacting the cells with a construct comprising one or more reporter genes under the control of a synapsin promoter, wherein an expression of the one or more reporter genes indicates that hiNSCs have differentiated into mature, functioning neurons.

18. (canceled)

19. (canceled)

20. (canceled)

21. The method of claim 12, wherein, prior to step (a), the method comprises
genetically modifying the hiNSCs to express a second reporter gene under the control of a glial fibrillary acidic protein (GFAP) promoter, a tumor necrosis factor (TNF)-α promoter, or a presenilin-2 (PSEN2) promoter.

22. The method of claim 12, wherein the hiNSCs are generated by:
(a) providing one or more human somatic cells;
(b) inducing transient expression of OCT4, KLF4, SOX2, and cMYC in the one or more human somatic cells for 2-6 days forming reprogrammed somatic cells;
(c) providing a plurality of inactivated embryonic fibroblasts; and
(d) contacting the reprogrammed somatic cells with the plurality of inactivated embryonic fibroblasts in a culture media comprising 20% KO DMEM xenogen-free serum replacement and at least 15 ng/ml recombinant bFGF to generate human induced neural stem cells (hiNSCs).

23. (canceled)

24. A genetically modified human induced neural stem cell (hiNSC) comprising: a viral vector encoding at least one reporter gene under the control of a synapsin promoter, wherein an expression of the at least one reporter gene indicates that the hiNSC has differentiated into a mature, functioning neuron.

25. (canceled)

26. A method of screening for a compound that alters an AD-associated phenotype, the method comprising:
(a) contacting the in vitro model of AD of claim 1 with a compound; and
(b) detecting a change in one or more AD-associated phenotype in the in vitro model of AD.

27.-38. (canceled)


SEQUENCE LISTING (31 sequences)

SEQ ID NO: 1 - DNA, 485 bp, Homo sapiens
  source 1..485: mol_type = other DNA
gtgtctagactgcagagggccctgcgtatgagtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcagtcgagaa

SEQ ID NO: 2 - DNA, 908 bp, synthetic construct
  misc_feature 1..908: note = Synthetic- Genetically encoded Ca2+indicator rGECO
  source 1..908: mol_type = other DNA
gcgggagtctggtgagcaagggcgaggaggataacatggccatcatcaaggagttcatgcgcttcaaggtgcacatggagggctccgtgaacggccacgagttcgagatcgagggcgagggcgagggccgcccctacgaggcctttcagaccgctaagctgaaggtgaccaagggtggccccctgcccttcgcctgggacatcctgtcccctcagttcatgtacggctccaaggcctacattaagcacccagccgacatccccgactacttcaagctgtccttccccgagggcttcaggtgggaccgcgtgatgaacttcgaggacggcggcattattcacgtgaaccaggactcctccctgcaggacggcgtattcatctacaaggtgaagctgcgcggcaccaacttcccccccgacggccccgtaatgcagaagaagaccatgggctgggaggctacgcgtgacgacctgactgaagagcagatcgcagaatttaaagaggctttctccctatttgacaaggacggggatgggacgataacaaccaaggagcttgggacggtgttccgatcgctggggcagaaccccacagaagcagagctgcaggacatgatcaatgaagtagatgccgacggtgacggcacattcgacttccctgagttcctgacgatgatggcaagaaaaatgaatgacacagacagtgaagaggaaattcgcgaagcgttccgcgtgtttgataaggacggcaatggctacatcggcgcagcagagcttcgccacgtgatgacagaccttggagagaagttaacagatgaggaggttgatgaaatgatcagggtagcagacatcgatggggatggtcaggtaaactacgaagagtttgtacaaatgatgacagcgaagtag

SEQ ID NO: 3 - DNA, 2209 bp, synthetic construct
  misc_feature 1..2209: note = Synthetic- Glial fibrillary acidic protein (GFAP) promoter
  source 1..2209: mol_type = other DNA
cccacctccctctctgtgctgggactcacagagggagacctcaggaggcagtctgtccatcacatgtccaaatgcagagcataccctgggctgggcgcagtggcgcacaactgtaattccagcactttgggaggctgatgtggaaggatcacttgagcccagaagttctagaccagcctgggcaacatggcaagaccctatctctacaaaaaaagttaaaaaatcagccacgtgtggtgacacacacctgtagtcccagctattcaggaggctgaggtgaggggatcacttaaggctgggaggttgaggctgcagtgagtcgtggttgcgccactgcactccagcctgggcaacagtgagaccctgtctcaaaagacaaaaaaaaaaaaaaaaaaaaaaagaacatatcctggtgtggagtaggggacgctgctctgacagaggctcgggggcctgagctggctctgtgagctggggaggaggcagacagccaggccttgtctgcaagcagacctggcagcattgggctggccgccccccagggcctcctcttcatgcccagtgaatgactcaccttggcacagacacaatgttcggggtgggcacagtgcctgcttcccgccgcaccccagcccccctcaaatgccttccgagaagcccattgagcagggggcttgcattgcaccccagcctgacagcctggcatcttgggataaaagcagcacagccccctaggggctgcccttgctgtgtggcgccaccggcggtggagaacaaggctctattcagcctgtgcccaggaaaggggatcaggggatgcccaggcatggacagtgggtggcagggggggagaggagggctgtctgcttcccagaagtccaaggacacaaatgggtgaggggactgggcagggttctgaccctgtgggaccagagtggagggcgtagatggacctgaagtctccagggacaacagggcccaggtctcaggctcctagttgggcccagtggctccagcgtttccaaacccatccatccccagaggttcttcccatctctccaggctgatgtgtgggaactcgaggaaataaatctccagtgggagacggaggggtggccagggaaacggggcgctgcaggaataaagacgagccagcacagccagctcatgtgtaacggctttgtggagctgtcaaggcctggtctctgggagagaggcacagggaggccagacaaggaaggggtgacctggagggacagatccaggggctaaagtcctgataaggcaagagagtgccggccccctcttgccctatcaggacctccactgccacatagaggccatgattgacccttagacaaagggctggtgtccaatcccagcccccagccccagaactccagggaatgaatgggcagagagcaggaatgtgggacatctgtgttcaagggaaggactccaggagtctgctgggaatgaggcctagtaggaaatgaggtggcccttgagggtacagaacaggttcattcttcgccaaattcccagcaccttgcaggcacttacagctgagtgagataatgcctgggttatgaaatcaaaaagttggaaagcaggtcagaggtcatctggtacagcccttccttccctttttttttttttttttttgtgagacaaggtctctctctgttgcccaggctggagtggcgcaaacacagctcactgcagcctcaacctactgggctcaagcaatcctccagcctcagcctcccaaagtgctgggattacaagcatgagccaccccactcagccctttccttcctttttaattgatgcataataattgtaagtattcatcatggtccaaccaaccctttcttgacccaccttcctagagagagggtcctcttgcttcagcggtcagggccccagacccatggtctggctccaggtaccacctgcctcatgcaggagttggcgtgcccaggaagctctgcctctgggcacagtgacctcagtggggtgaggggagctctccccatagctgggctgcggcccaaccccaccccctcaggctatgccagggggtgttgccaggggcacccgggcatcgccagtctagcccactccttcataaagccctcgcatcccaggagcgagcagagccagagcaggttggagaggagacgcatcacctccgctgctcgccggg

SEQ ID NO: 4 - DNA, 21 bp, synthetic construct
  misc_feature 1..21: note = Synthetic- Amyloid precursor protein (APP) primer
  source 1..21: mol_type = other DNA
gtctctctccctgctctacaa

SEQ ID NO: 5 - DNA, 22 bp, synthetic construct
  misc_feature 1..22: note = Synthetic- Amyloid precursor protein (APP) primer
  source 1..22: mol_type = other DNA
ggccaagacgtcatctgaatag

SEQ ID NO: 6 - DNA, 19 bp, synthetic construct
  misc_feature 1..19: note = Synthetic- Beta secretase 1 (BACE1) primer
  source 1..19: mol_type = other DNA
ccatccttccgcagcaata

SEQ ID NO: 7 - DNA, 22 bp, synthetic construct
  misc_feature 1..22: note = Synthetic- Beta secretase 1 (BACE1) primer
  source 1..22: mol_type = other DNA
cgtagaagccctccatgataac

SEQ ID NO: 8 - DNA, 19 bp, synthetic construct
  misc_feature 1..19: note = Synthetic- Tumor necrosis factor alpha (TNF) primer
  source 1..19: mol_type = other DNA
gaggccaagccctggtatg

SEQ ID NO: 9 - DNA, 19 bp, synthetic construct
  misc_feature 1..19: note = Synthetic- Tumor necrosis factor alpha (TNF)
  source 1..19: mol_type = other DNA
cgggccgattgatctcagc

SEQ ID NO: 10 - DNA, 21 bp, synthetic construct
  misc_feature 1..21: note = Synthetic- Serpina3n (SERP3) primer
  source 1..21: mol_type = other DNA
gctcatcaacgactacgtgaa

SEQ ID NO: 11 - DNA, 23 bp, synthetic construct
  misc_feature 1..23: note = Synthetic- Serpina3n (SERP3) primer
  source 1..23: mol_type = other DNA
caccattacccactttttcttgc

SEQ ID NO: 12 - DNA, 20 bp, synthetic construct
  misc_feature 1..20: note = Synthetic- Lipocalin 2 (LCN2)
  source 1..20: mol_type = other DNA
gacaaccaattccaggggaa

SEQ ID NO: 13 - DNA, 21 bp, synthetic construct
  misc_feature 1..21: note = Synthetic- Lipocalin 2 (LCN2) primer
  source 1..21: mol_type = other DNA
gcatacatcttttgcgggtct

SEQ ID NO: 14 - DNA, 23 bp, synthetic construct
  misc_feature 1..23: note = Synthetic- Presenilin 1 (PSEN1) primer
  source 1..23: mol_type = other DNA
tggctaccattaagtcagtcagc

SEQ ID NO: 15 - DNA, 21 bp, synthetic construct
  misc_feature 1..21: note = Synthetic- Presenilin 1 (PSEN1) primer
  source 1..21: mol_type = other DNA
cccacagtctcggtatcttct

SEQ ID NO: 16 - DNA, 22 bp, synthetic construct
  misc_feature 1..22: note = Synthetic- Presenilin 2 (PSEN2) primer
  source 1..22: mol_type = other DNA
ctgaccgctatgtctgtagtgg

SEQ ID NO: 17 - DNA, 21 bp, synthetic construct
  misc_feature 1..21: note = Synthetic- Presenilin 2 (PSEN2) primer
  source 1..21: mol_type = other DNA
cttcgctccgtatttgagggt

SEQ ID NO: 18 - DNA, 23 bp, synthetic construct
  misc_feature 1..23: note = Synthetic- Interleukin 6 (IL- 6) primer
  source 1..23: mol_type = other DNA
tcaatattagagtctcaaccccc

SEQ ID NO: 19 - DNA, 20 bp, synthetic construct
  misc_feature 1..20: note = Synthetic- Interleukin 6 (IL- 6) primer
  source 1..20: mol_type = other DNA
ttgttttctgccagtgcctc

SEQ ID NO: 20 - DNA, 20 bp, synthetic construct
  misc_feature 1..20: note = Synthetic- Interleukin 1 beta (IL1B) primer
  source 1..20: mol_type = other DNA
cagaagtacctgagctcgcc

SEQ ID NO: 21 - DNA, 20 bp, synthetic construct
  misc_feature 1..20: note = Synthetic- Interleukin 1 beta (IL1B) primer
  source 1..20: mol_type = other DNA
agattcgtagctggatgccg

SEQ ID NO: 22 - DNA, 20 bp, synthetic construct
  misc_feature 1..20: note = Synthetic- Interferon gamma (IFNy) primer
  source 1..20: mol_type = other DNA
actgtcgccagcagctaaaa

SEQ ID NO: 23 - DNA, 20 bp, synthetic construct
  misc_feature 1..20: note = Synthetic- Interferon gamma (IFNy) primer
  source 1..20: mol_type = other DNA
tattgcaggcaggacaacca

SEQ ID NO: 24 - DNA, 20 bp, synthetic construct
  misc_feature 1..20: note = Synthetic- Vimentin (VIM) primer
  source 1..20: mol_type = other DNA
tccgcacattcgagcaaaga

SEQ ID NO: 25 - DNA, 20 bp, synthetic construct
  misc_feature 1..20: note = Synthetic- Vimentin (VIM) primer
  source 1..20: mol_type = other DNA
tgattcaagtctcagcgggc

SEQ ID NO: 26 - DNA, 19 bp, synthetic construct
  misc_feature 1..19: note = Synthetic- Glyceraldehyde 3-phosphate dehydrogenase (GAPDH) primer
  source 1..19: mol_type = other DNA
attgccctcaacgaccact

SEQ ID NO: 27 - DNA, 19 bp, synthetic construct
  misc_feature 1..19: note = Synthetic- Glyceraldehyde 3-phosphate dehydrogenase (GAPDH) primer
  source 1..19: mol_type = other DNA
atgaggtccaccaccctgt

SEQ ID NO: 28 - DNA, 20 bp, synthetic construct
  misc_feature 1..20: note = Synthetic- Glial fibrillary acidic protein (GFAP) primer
  source 1..20: mol_type = other DNA
actggcagagcttgttagtg

SEQ ID NO: 29 - DNA, 20 bp, synthetic construct
  misc_feature 1..20: note = Synthetic- Glial fibrillary acidic protein (GFAP) primer
  source 1..20: mol_type = other DNA
agtgacaggaagaggtgaga

SEQ ID NO: 30 - DNA, 20 bp, synthetic construct
  misc_feature 1..20: note = Synthetic- Human alpha- herpesvirus 1 strain MacIntyre primer
  source 1..20: mol_type = other DNA
tggcttttcggactacaccc

SEQ ID NO: 31 - DNA, 20 bp, synthetic construct
  misc_feature 1..20: note = Synthetic- Human alpha- herpesvirus 1 strain MacIntyre primer
  source 1..20: mol_type = other DNA
ttcgaaggccgtgaacgtaa